SEQUENCE LISTING (38 sequences)

SEQ ID NO: 1 - DNA, 243 bp, Homo sapiens
  source 1..243: mol_type = genomic DNA
atggaatcccccaagaagaagaaccagcagctgaaggtcggaatcctgcacctgggttcccgtcagaagaagatccgtatccagctgcgttcccagtgcgctacctggaaggtcatctgcaagtcctgcatctcccagacccccggtatcaacctggacctgggctccggtgtcaaggtcaagatcatccccaaggaagaacactgcaagatgcccgaggctggcgaggaacagccccaggtg

SEQ ID NO: 2 - DNA, 1200 bp, Homo sapiens
  source 1..1200: mol_type = genomic DNA
atggggactcctgcttctggaaggaaaagaacacctgtgaaagaccgattttctgcagaagatgaagctttgagtaacattgccagagaggcagaggcaaggctggcagcaaaacgggctgcccgggcagaagcaagagatatacgcatgagagaactggaacgacaacaaaaagagttggatgaaaaatctgacaaacagtatgctgaaaattatacaagaccttcatctcgaaattctgcctcagcaacaacccctctaagtggaaactcatccagacgaggaagtggggacaccagcagcttaatagatccagacacttcattaagtgaattgcgggaatctttgtctgaagtggaagaaaaatacaagaaagccatggtttccaatgcacagttagacaatgagaagaacaatttgatctaccaagtagacacactcaaggatgttattgaagagcaggaggaacagatggcagaattttatagagaaaatgaagaaaaatcaaaggagttagaaaggcagaaacatatgtgtagtgtgctgcagcataagatggaagaacttaaagaaggcctgcggcaaagagatgagcttattgagaaacatggcttagttataatccccgatggcactcccaatggtgatgtcagtcatgaaccagtggctggagccatcactgttgtgtctcaggaagctgctcaggtcttggagtcagcaggagaagggccattagatgtaaggctacgaaaacttgctggagagaaggaagaactactgtcacagattagaaaactgaagcttcagttagaggaggaacgacagaaatgctccaggaatgatggcacagtgggtgacctggcaggactgcagaatggctcagacttgcagttcatcgaaatgcagagagatgccaatagacaaattagcgaatacaaatttaagctttcaaaagcagaacaggatataactaccttggagcaaagtattagccggcttgagggacaggttctgagatataaaactgctgctgagaatgctgagaaagttgaagatgaattgaaagcagaaaaacggaagctacaacgagagttacgaacagcactggacaagattgaggagatggagatgaccaacagccacctggccaagcggctggagaagatgaaggccaataggacagcacttctggcccagcag

SEQ ID NO: 3 - DNA, 348 bp, Homo sapiens
  source 1..348: mol_type = genomic DNA
atgtcctggcgtggtcgttccacctaccgtccccgtcctcgtcgttacgtcgagccccccgagatgatcggtcccatgcgtcccgagcagttctccgacgaggtcgagcccgctacccccgaggaaggcgagcctgctactcagcgtcaggaccccgctgctgctcaagagggcgaggacgagggcgcttccgctggccagggtcctaagcccgaggctcactcccaagagcagggtcacccccagaccggttgcgagtgcgaggacggtcccgacggtcaagagatggacccccccaaccctgaggaagtcaagacccccgaagagggcgaaaagcagtcccagtgc

SEQ ID NO: 4 - DNA, 1893 bp, Homo sapiens
  source 1..1893: mol_type = genomic DNA
atgtcccactgggctcccgagtggaagcgtgctgaggctaacccccgtgacctgggcgcttcttgggacgtgcgcggttcccgtggtagcggttggagcggtcccttcggtcaccaaggtccccgtgctgctggttcccgcgagccccccctgtgcttcaagatcaagaacaacatggtcggagtggtcatcggttactccggttccaagatcaaggacctgcagcactccaccaacaccaagatccagatcatcaacggcgagtccgaggctaaggtccgcatcttcggcaaccgcgagatgaaggctaaggccaaggctgctatcgagactctgatccgcaagcaagagtcctacaactccgagtcctccgtggacaacgctgcttcccagacccccatcggtcgtaacctgggtcgtaacgacatcgtgggcgaggctgagcccctgtccaactgggaccgtatccgcgctgctgtggtcgagtgcgagaagcgcaagtgggctgacctgccccccgtgaagaagaacttctacatcgagtccaaggctacctcctgcatgtccgagatgcaagtgatcaactggcgcaaggaaaacttcaacatcacttgcgacgacctgaagtccggcgagaagcgtctgatccccaagcctacctgccgtttcaaggacgctttccagcagtaccccgacctgctgaagtccatcatccgtgtgggtatcgtgaagcccacccccatccagtcccaggcttggccaatcatcctgcagggtatcgacctgatcgtggtggctcagaccggcaccggcaagaccctgtcctacctgatgcccggtttcatccacctggactcccagcccatctcccgcgagcagcgtaacggtcccggcatgctggtgctgacccctacccgtgaactggctctgcacgtcgaggctgagtgctccaagtactcctacaagggcctgaagtctatctgcatctacggtggtcgtaaccgtaacggccagatcgaggacatctccaagggtgtcgacatcatcattgctacccccggtcgtctgaacgacctgcagatgaacaactccgtgaacctgcgttccatcacctacctggtcatcgacgaggctgacaagatgctggacatggagttcgagccccagatccgcaagatcctgctggacgtgcgtcccgaccgtcagaccgtgatgacctccgctacctggcccgacaccgtgcgtcagctggctctgtcttacctgaaggaccccatgatcgtgtacgtgggcaacctgaacctggtggctgtgaacaccgtgaagcagaacatcatcgtgaccaccgagaaggaaaagagggctctgacccaagagttcgtcgagaacatgtcccccaacgacaaggtcatcatgttcgtgtcccagaagcacattgctgacgacctgtcctccgatttcaacatccaaggcatctccgctgagtccctgcacggcaactccgagcagtccgaccaagagcgtgctgtcgaggacttcaagtccggcaacatcaagatcctcatcaccaccgacatcgtgtcccgtggcctggacctgaacgacgtgacccacgtgtacaactacgacttcccccgtaacatcgacgtgtacgtgcaccgtgtgggttacatcggtcgcaccggaaagaccggaacctccgtgaccctgatcacccagcgcgactccaagatggctggcgagctgatcaagatcttggaccgtgctaaccagtccgtgcccgaggacctggtggtcatggctgagcagtacaagctgaaccagcagaagcgtcaccgcgagactcgttcccgcaagcccggacagcgtcgcaaggagttctacttcctgtcc

SEQ ID NO: 5 - DNA, 1944 bp, Homo sapiens
  source 1..1944: mol_type = genomic DNA
atgtcccaccacggtggtgctcccaaggcttccacctgggtggtggcttcccgtcgttcctccaccgtgtcccgtgctcccgagcgtcgtcccgctgaggaactgaaccgtaccggtcccgagggctactccgtgggtcgtggtggtcgttggcgtggcacctctcgtccccctgaggctgtggctgctggtcacgaggaactgcccctgtgcttcgctctgaagtcccacttcgtgggtgctgtgatcggtcgcggtggttccaagatcaagaacatccagtccaccaccaacaccaccatccagatcatccaagagcagcccgagtccctggtcaagatcttcggttccaaggctatgcagaccaaggctaaggctgtgatcgacaacttcgtgaagaagctggaagagaactacaactccgagtgcggtatcgacaccgctttccagccctccgtgggcaaggacggttccaccgacaacaacgtggtggctggcgaccgtcccctgatcgactgggaccagatccgtgaagagggcctgaagtggcaaaagaccaagtgggctgacctgccccccatcaagaagaacttctacaaggaatccaccgctacctccgctatgtccaaggtcgaggctgactcctggcgcaaggaaaacttcaacatcacctgggacgacctgaaggacggcgagaagcgtcccatccccaaccctacctgcaccttcgacgacgctttccagtgctaccccgaagtgatggaaaacatcaagaaggctggtttccagaagcccacccccatccagtcccaggcttggcccatcgtgctgcagggtatcgacctgatcggtgtcgctcagaccggcaccggcaagaccctgtgctacctgatgcccggtttcatccacctggtgctgcagccctccctgaagggccagcgtaaccgtcccggcatgctggtgctgacccctacccgcgaactggctctgcaggtcgagggcgagtgctgcaagtactcctacaagggcctgcgttccgtgtgcgtgtacggtggtggcaaccgtgacgagcagatcgaggaactcaagaagggtgtcgacatcatcatcgctacccccggtcgtctgaacgacctgcagatgtccaacttcgtcaacctgaagaacatcacctacctggtcctggacgaggctgacaagatgctggacatgggtttcgagccccagatcatgaagatcctgctggacgtgcgtcccgaccgtcagaccgtgatgacctccgctacctggccccactccgtgcaccgtctggctcagtcctacctgaaggaacccatgatcgtgtacgtgggcaccctggacctggtggctgtgtcctccgtgaagcagaacatcatcgtgaccaccgaggaagagaagtggtcccacatgcagactttcttgcagtccatgtcctctaccgacaaggtcatcgtgttcgtgtcccgcaaggctgtcgctgaccacctgtcctccgacctgatcctgggcaacatctccgtcgagtccctgcacggcgaccgcgagcagcgtgaccgcgagaaggctctcgagaacttcaagaccggcaaggtccgcatcctgatcgctaccgacctggcttcccgcggactggacgtgcacgacgtgacccacgtgtacaacttcgacttcccccgtaacatcgaggaatacgtgcaccgtatcggtcgtaccggtcgtgctggtcgcaccggtgtctccatcaccaccctgacccgtaacgactggcgtgtggcttccgagctgatcaacatcctcgagcgtgctaaccagtccatccccgaggaactggtgtctatggctgagcgtttcaaggctcaccagcaaaagcgcgagatggaacgcaagatggaacgtccccagggtcgtcccaagaagttccac

SEQ ID NO: 6 - DNA, 351 bp, Homo sapiens
  source 1..351: mol_type = genomic DNA
atgtcctggcgtggtcgttccacctactactggccccgtccccgtcgttacgtgcagccccccgagatgatcggtcccatgcgtcccgagcagttctccgacgaggtcgagcccgctacccccgaggaaggcgagcctgctactcagcgtcaggaccccgctgctgctcaagagggcgaggacgagggcgcttccgctggccagggtcctaagcccgaggctgactcccaagagcagggtcacccccagaccggttgcgagtgcgaggacggtcccgacggtcaagagatggacccccccaaccctgaggaagtcaagacccccgaagagggcgaaggccagtcccagtgc

SEQ ID NO: 7 - DNA, 1107 bp, Homo sapiens
  source 1..1107: mol_type = genomic DNA
atgccccgtgctcccaagcgtcagcgttgcatgcccgaagaggacctgcagtcccagtccgagactcagggcctcgagggtgctcaggctcccctggctgtggaagaggacgcttccagctctacctctacctcctccagcttccccagcagcttcccatcctccagctcctctagctcctcctcctgctaccccctgatcccctccacccccgaggaagtgtccgctgacgacgagactcccaaccccccccagtccgctcagatcgcttgctcctccccctccgtggtggcttccctgcctctggaccagtccgacgagggttccagctcccagaaggaagagtcccccagcaccctgcaggtcctgcccgactccgagtccctgccccgttccgagatcgacgagaaggttacagacctggtgcagttcctgctgttcaagtaccagatgaaggaacccatcaccaaggctgagatcctcgagtccgtgatcaagaactacgaggaccacttccccctgctgttctccgaggcttccgagtgcatgctgctggtgttcggtatcgacgtgaaggaagtggaccctaccggtcactccttcgtgctggtcacctccctgggcctgacctacgacggcatgctgtccgacgtgcagtccatgcccaagaccggtatcctgatcctcatcctgtccatcatcttcatcgagggctactgcactcctgaggaagtgatctgggaggctctgaacatgatgggcctgtacgacggaatggaacacctgatctacggcgagccccgcaagctgctgacccaggactgggtgcaagagaactacctcgagtaccgtcaggtgcccggttccgaccccgctcgttacgagttcctgtggggtccccgtgctcacgctgagatccgcaagatgtccctgctgaagttcctggctaaggtcaacggctccgacccccgttccttcccactgtggtacgaggaagctctgaaggacgaggaagagagggctcaggaccgtatcgctaccaccgacgacaccaccgctatggcttccgcttcctctagcgctaccggttccttcagctaccccgag

SEQ ID NO: 8 - DNA, 378 bp, Homo sapiens
  source 1..378: mol_type = genomic DNA
atgtctgtcatggacctcgccaatacttgctccagctttcagtcggacctggatttctgttcagattgcggctcggtcctgcctctgcccggggctcaggatacggtcacctgtattcgctgtggcttcaacatcaacgttcgggactttgaggggaaggttgtgaagacttcggttgtgttccaccaactggggacagccatgcctatgtcggtggaggaagggcctgagtgccagggacctgtggttgacaggcgctgccctcgatgtggtcatgaaggaatggcataccacaccagacagatgcgttcagccgatgaagggcaaactgtcttctacacctgtaccaactgcaagttccaggagaaggaagactct

SEQ ID NO: 9 - DNA, 1344 bp, Homo sapiens
  source 1..1344: mol_type = genomic DNA
atgctgtggctagcccttggcccctttcctgccatggagaaccaggtgctggtaattcgcatcaagatcccaaatagtggcgcggtggactggacagtgcactccgggccgcagttactcttcagggatgtgctggatgtgataggccaggttctgcctgaagcaacaactacagcatttgaatatgaagatgaagatggtgatcgaattacagtgagaagtgatgaggaaatgaaggcaatgctgtcatattattattccacagtaatggaacagcaagtaaatggacagttaatagagcctctgcagatatttccaagagcctgcaagcctcctggggaacggaacatacatggcctgaaggtgaatactcgggccggaccctctcaacacagcagcccagcagtctcagattcacttccaagcaatagcttaaagaagtcttctgctgaactgaaaaaaatactagccaatggccagatgaatgaacaagacatacgatatcgggacactcttggtcatggcaacggaggcacagtctacaaagcatatcatgtcccgagtgggaaaatattagctgtaaaggtcatactactagatattacactggaacttcagaagcaaattatgtctgaattggaaattctttataagtgcgattcatcatatatcattggattttatggagcattttttgtagaaaacaggatttcaatatgtacagaattcatggatgggggatctttggatgtatataggaaaatgccagaacatgtccttggaagaattgcagtagcagttgttaaaggccttacttatttgtggagtttaaagattttacatagagacgtgaagccctccaatatgctagtaaacacaagaggacaggttaagctgtgtgattttggagttagcactcagctggtgaattctatagccaagacgtatgttggaacaaatgcttatatggcgcctgaaaggatttcaggggagcagtatggaattcattctgatgtctggagcttaggaatctcttttatggagcttgctcttgggaggtttccatatcctcagattcagaaaaaccagggatctttaatgcctctccagcttctgcagtgcattgttgatgaggattcgcccgtccttccagttggagagttctcggagccatttgtacatttcatcactcagtgtatgcgaaaacagccaaaagaaaggccagcacctgaagaattgatgggccacccgttcatcgtgcagttcaatgatggaaatgccgccgtggtgtccatgtgggtgtgccgggcgctggaggagaggcggagccagcaggggcccccg

SEQ ID NO: 10 - DNA, 951 bp, Homo sapiens
  source 1..951: mol_type = genomic DNA
atgtcttctgagcagaagagtcagcactgcaagcctgaggaaggcgttgaggcccaagaagaggccctgggcctggtgggtgcacaggctcctactactgaggagcaggaggctgctgtctcctcctcctctcctctggtccctggcaccctggaggaagtgcctgctgctgagtcagcaggtcctccccagagtcctcagggagcctctgccttacccactaccatcagcttcacttgctggaggcaacccaatgagggttccagcagccaagaagaggaggggccaagcacctcgcctgacgcagagtccttgttccgagaagcactcagtaacaaggtggatgagttggctcattttctgctccgcaagtatcgagccaaggagctggtcacaaaggcagaaatgctggagagagtcatcaaaaattacaagcgctgctttcctgtgatcttcggcaaagcctccgagtccctgaagatgatctttggcattgacgtgaaggaagtggaccccaccagcaacacctacacccttgtcacctgcctgggcctttcctatgatggcctgctgggtaataatcagatctttcccaagacaggccttctgataatcgtcctgggcacaattgcaatggagggcgacagcgcctctgaggaggaaatctgggaggagctgggtgtgatgggggtgtatgatgggagggagcacactgtctatggggagcccaggaaactgctcacccaagattgggtgcaggaaaactacctggagtaccggcaggtacccggcagtaatcctgcgcgctatgagttcctgtggggtccaagggctctggctgaaaccagctatgtgaaagtcctggagcatgtggtcagggtcaatgcaagagttcgcattgcctacccatccctgcgtgaagcagctttgttagaggaggaagagggagtc

SEQ ID NO: 11 - DNA, 2136 bp, Homo sapiens
  source 1..2136: mol_type = genomic DNA
atgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagtg

SEQ ID NO: 12 - DNA, 864 bp, Homo sapiens
  source 1..864: mol_type = genomic DNA
atgtccgctaccggcgaccgtcaccctacccagggcgaccaagaggctcccgtgtcccaagagggtgctcaggctgaggctgctggtgctggcaaccaggaaggtggcgactccggtcccgactcctccgacgtggtgcctgctgctgaggtcgtgggtgtcgctggtcctgtcgagggcctgggagaggaagagggcgagcaggctgctggcctggctgctgtgcctcgtggtggttccgctgaagaggactccgacatcggtcccgctaccgaggaagaggaagaagaagagggcaacgaggctgctaacttcgacctggctgtggtggctcgtcgttaccccgcttccggtatccacttcgtgctgctggacatggtgcactccctgctgcaccgtctgtcccacaacgaccacatcctgatcgagaaccgtcagctgtcccgtctgatggtcggaccccacgctgctgctcgtaacctgtggggcaacctgccccccctgctgctgcctcaacgtctgggagctggtgctgctgctagggctggcgagggactgggcctgatccaagaggctgcttccgtgcccgagcccgctgtgcctgctgacttggctgagatggcccgcgagcctgctgaggaagctgctgaagagaagctgtccgaggaagccaccgaggaacccgacgctgaggaaccagctactgaggaacccaccgctcaagaggctaccgctcctgaggaagtgaccaagtcccagcccgagaagtgggacgaggaagctcaggacgctgctggcgaggaagagaaggaacaagaaaaggaaaaggacgccgagaacaaggtcaagaactccaagggcacc

SEQ ID NO: 13 - DNA, 1737 bp, Homo sapiens
  source 1..1737: mol_type = genomic DNA
atgaacaagctgtacatcggcaacctgtccgagaacgctgctccctccgacctcgagtccatcttcaaggacgctaagatccccgtgtccggacccttcctggtcaagaccggttacgctttcgtggactgccccgacgagtcctgggctctgaaggctatcgaggctctgtccggcaagatcgagctgcacggcaagcccatcgaggtcgagcactccgtgcccaagcgtcagcgtatccgcaagctgcagatccgtaacatccccccacacctccagtgggaggtgctggactccctgctggtgcagtacggtgtcgtcgagtcctgcgagcaagtgaacaccgactccgagactgctgtggtcaacgtgacctactcctccaaggaccaggctcgtcaggctctggacaagctgaacggtttccagctcgagaacttcaccctgaaggtggcctacatccccgacgagatggctgctcagcagaaccccctgcagcagccccgtggtcgtcgtggactgggacagcgtggtagctcccgtcagggttcccccggttccgtgtccaagcagaagccctgcgacctgcccctgcgtctgctggtgcctacccagttcgtgggtgctatcatcggcaaggaaggtgctaccatccgcaacatcaccaagcagacccagtccaagatcgacgtccaccgcaaggaaaacgctggcgctgctgagaagtccatcaccatcctgtccacccccgagggcacctccgctgcttgcaagtccatcctcgagatcatgcacaaggaagcccaggacatcaagttcaccgaggaaatccccctgaagatcctggctcacaacaacttcgtgggtcgtctgatcggaaaggaaggccgtaacctgaagaagatcgagcaggacaccgacaccaagatcaccatctccccactgcaagagctgaccctgtacaaccccgagcgtaccatcaccgtgaagggcaacgtggaaacctgcgctaaggctgaagaggaaatcatgaagaagatccgcgagtcctacgagaacgatatcgcttccatgaacctgcaggctcacctgatccccggcctgaacctgaacgctctgggcctgttcccccctacctccggcatgcctcctcccacctctggtcccccctccgctatgacccccccatacccccagttcgagcagtccgagactgagactgtgcacctgttcatccccgctctgtccgtcggtgccatcatcggaaagcagggccagcacatcaagcagctgtcccgtttcgctggtgcttccatcaagatcgctcccgctgaggctcccgacgctaaggtccgcatggtcatcatcaccggtccccccgaggctcagttcaaggctcagggtcgtatctacggcaagatcaaggaagagaacttcgtcagccccaaggaagaagtgaagctcgaggctcacatccgtgtgccatccttcgctgctggtcgtgttatcggcaagggtggcaagaccgtgaacgagctgcagaacctgtcctccgctgaggtggtggtgccccgtgaccagacccctgacgagaacgaccaggtggtggtcaagatcaccggtcacttctacgcttgccaggtggcccagcgcaagatccaagagatcctgacccaagtgaagcagcaccagcagcagaaggctctgcagtccggtccccctcagtcccgtcgcaag

SEQ ID NO: 14 - DNA, 630 bp, Homo sapiens
  source 1..630: mol_type = genomic DNA
atgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgagaggaggcgccccgcggggtccgcatggcggtgccgcttctgcgcaggatggaaggtgcccctgcggggccaggaggccggacagccgcctgcttgagttgcacatcacgatgcctttctcgtcgcccatggaagcggagctggtccgcaggatcctgtcccgggatgccgcaccgctcccccgaccaggggcggttctgaaggacttcaccgtgtccggcaacctactgtttatgtcagttcgggaccaggacagggaaggcgctgggcggatgagggtggtgggttgggggctgggatccgcctccccggaggggcagaaagctagagatctcagaacacccaaacacaaggtctcagaacagagacctggtacaccaggcccgccgccacccgagggagcccagggagatgggtgcagaggtgtcgcctttaatgtgatgttctctgcccctcacatt

SEQ ID NO: 15 - DNA, 1227 bp, Homo sapiens
  source 1..1227: mol_type = genomic DNA
atgcaggtcaccctgaagaccctccagcagcagaccttcaagatagacattgaccccgaggagacggtgaaagcactgaaagagaagattgaatctgaaaaggggaaagatgcctttccagtagcaggtcaaaaattaatttatgcaggcaaaatcctcaatgatgatactgctctcaaagaatataaaattgatgagaaaaactttgtggtggttatggtgaccaaacccaaagcagtgtccacaccagcaccagctacaactcagcagtcagctcctgccagcactacagcagttacttcctccaccaccacaactgtggctcaggctccaacccctgtccctgccttggcccccacttccacacctgcatccatcactccagcatcagcgacagcatcttctgaacctgcacctgctagtgcagctaaacaagagaagcctgcagaaaagccagcagagacaccagtggctactagcccaacagcaactgacagtacatcgggtgattcttctcggtcaaacctttttgaagatgcaacgagtgcacttgtgacgggtcagtcttacgagaatatggtaactgagatcatgtcaatgggctatgaacgagagcaagtaattgcagccctgagagccagtttcaacaaccctgacagagcagtggagtatcttttaatgggaatccctggagatagagaaagtcaggctgtggttgacccccctcaagcagctagtactggggttcctcagtcttcagcagtggctgcagctgcagcaactacgacagcaacaactacaacaacaagttctggaggacatccccttgaatttttacggaatcagcctcagtttcaacagatgagacaaattattcagcagaatccttccttgcttccagcgttactacagcagataggtcgagagaatcctcaattacttcagcaaattagccaacaccaggagcattttattcagatgttaaatgaaccagttcaagaagctggtggtcaaggaggaggaggtggaggtggcagtggaggaattgcagaagctggaagtggtcatatgaactacattcaagtaacacctcaggaaaaagaagctatagaaaggttaaaggcattaggatttcctgaaggacttgtgatacaagcgtattttgcttgtgagaagaatgagaatttggctgccaattttcttctacagcagaactttgatgaagat

SEQ ID NO: 16 - DNA, 624 bp, Homo sapiens
  source 1..624: mol_type = genomic DNA
atggacccgttcctggtgctgctgcactcggtgtcgtccagcctgtcgagcagcgagctgaccgagctcaagttcctatgcctcgggcgcgtgggcaagcgcaagctggagcgcgtgcagagcggcctagacctcttctccatgctgctggagcagaacgacctggagcccgggcacaccgagctcctgcgcgagctgctcgcctccctgcggcgccacgacctgctgcggcgcgtcgacgacttcgaggcgggggcggcggccggggccgcgcctggggaagaagacctgtgtgcagcatttaacgtcatatgtgataatgtggggaaagattggagaaggctggctcgtcagctcaaagtctcagacaccaagatcgacagcatcgaggacagatacccccgcaacctgacagagcgtgtgcgggagtcactgagaatctggaagaacacagagaaggagaacgcaacagtggcccacctggtgggggctctcaggtcctgccagatgaacctggtggctgacctggtacaagaggttcagcaggcccgtgacctccagaacaggagtggggccatgtccccgatgtcatggaactcagacgcatctacctccgaagcgtcc

SEQ ID NO: 17 - DNA, 678 bp, Homo sapiens
  source 1..678: mol_type = genomic DNA
atgtcctccccccgtgacttccgtgctgagcccgtgaacgactacgagggcaacgactccgaggctgaggacctgaacttccgtgaaaccctgccctccagctcccaagagaacaccccccgttccaaggtgttcgagaacaaggtcaactccgagaaggtcaagctgtccctgcgcaacttcccccacaacgattacgaggacgtgttcgaggaaccctccgagtccggttccgacccctccatgtggaccgctcgtggtcccttccgtcgtgaccgttggtcctccgaggacgaggaagctgctggaccctcccaggctctgtcccccctgctgtccgacacccgcaagatcgtgtccgagggcgagctggaccagctggctcagatccgtcccctgatcttcaacttccacgagcagaccgctatcaaggactgcctgaagatcctcgaggaaaagaccgctgcttacgacatcatgcaagagttcatggctctcgagctgaagaacctgcccggcgagttcaactccggcaaccagccctccaaccgcgagaagaaccgttaccgtgacatcctgcctttccagcaccacggttactccggtcccaacgagcgtaccaccttctggcacggttccaacgagggtgctgtgtccctgctgctgcgctactgcgct

SEQ ID NO: 18 - DNA, 852 bp, Homo sapiens
  source 1..852: mol_type = genomic DNA
atggacgccatcaagaagaagatgcagatgctgaagctcgacaaggagaacgccttggatcgagctgagcaggcggaggccgacaagaaggcggcggaagacaggagcaagcagctggaagatgagctggtgtcactgcaaaagaaactcaagggcaccgaagatgaactggacaaatattctgaggctctcaaagatgcccaggagaagctggagctggcagagaaaaaggccaccgatgctgaagccgacgtagcttctctgaacagacgcatccagctggttgaggaagagttggatcgtgcccaggagcgtctggcaacagctttgcagaagctggaggaagctgagaaggcagcagatgagagtgagagaggcatgaaagtcattgagagtcgagcccaaaaagatgaagaaaaaatggaaattcaggagatccaactgaaagaggcaaagcacattgctgaagatgccgaccgcaaatatgaagaggtggcccgtaagctggtcatcattgagagcgacctggaacgtgcagaggagcgggctgagctctcagaaggccaagtccgacagctggaagaacaattaagaataatggatcagaccttgaaagcattaatggctgcagaggataagtactcgcagaaggaagacagatatgaggaagagatcaaggtcctttccgacaagctgaaggaggctgagactcgggctgagtttgcggagaggtcagtaactaaattggagaaaagcattgatgacttagaagacgagctgtacgctcagaaactgaagtacaaagccatcagcgaggagctggaccacgctctcaacgatatgacttccatg

SEQ ID NO: 19 - DNA, 540 bp, Homo sapiens
  source 1..540: mol_type = genomic DNA
atgcaggctgagggtcgtggcaccggtggttccactggcgacgctgacggtcccggtggtcctggtatccccgacggtcctggtggcaacgctggtggtccaggcgaggctggtgctaccggtggtcgtggtcctcgtggtgctggtgctgctcgtgcttccggtccaggtggtggtgctccccgtggtcctcacggtggtgctgcttccggcctgaacggttgctgccgttgcggtgctcgcggtcccgagtcccgtctgctcgagttctacctggctatgcccttcgctacccctatggaagctgagctggctcgtcgttccctggctcaggacgctcctcctctgcccgtgcccggtgtcctgctgaaggagttcactgtctccggcaacatcctgaccatccgtctgaccgctgctgaccaccgtcagctccagctgtccatctcctcatgcctgcagcagctgtccctgctgatgtggatcacccagtgtttcttgcccgtgttcctggctcagcccccctccggtcaacgtcgt

SEQ ID NO: 20 - protein, 81 aa, Homo sapiens
MESPKKKNQQLKVGILHLGSRQKKIRIQLRSQCATWKVICKSCISQTPGINLDLGSGVKVKIIPKEEHCKMPEAGEEQPQV

SEQ ID NO: 21 - protein, 400 aa, Homo sapiens
MGTPASGRKRTPVKDRFSAEDEALSNIAREAEARLAAKRAARAEARDIRMRELERQQKELDEKSDKQYAENYTRPSSRNSASATTPLSGNSSRRGSGDTSSLIDPDTSLSELRESLSEVEEKYKKAMVSNAQLDNEKNNLIYQVDTLKDVIEEQEEQMAEFYRENEEKSKELERQKHMCSVLQHKMEELKEGLRQRDELIEKHGLVIIPDGTPNGDVSHEPVAGAITVVSQEAAQVLESAGEGPLDVRLRKLAGEKEELLSQIRKLKLQLEEERQKCSRNDGTVGDLAGLQNGSDLQFIEMQRDANRQISEYKFKLSKAEQDITTLEQSISRLEGQVLRYKTAAENAEKVEDELKAEKRKLQRELRTALDKIEEMEMTNSHLAKRLEKMKANRTALLAQQ

SEQ ID NO: 22 - protein, 116 aa, Homo sapiens
MSWRGRSTYRPRPRRYVEPPEMIGPMRPEQFSDEVEPATPEEGEPATQRQDPAAAQEGEDEGASAGQGPKPEAHSQEQGHPQTGCECEDGPDGQEMDPPNPEEVKTPEEGEKQSQC

SEQ ID NO: 23 - protein, 631 aa, Homo sapiens
MSHWAPEWKRAEANPRDLGASWDVRGSRGSGWSGPFGHQGPRAAGSREPPLCFKIKNNMVGVVIGYSGSKIKDLQHSTNTKIQIINGESEAKVRIFGNREMKAKAKAAIETLIRKQESYNSESSVDNAASQTPIGRNLGRNDIVGEAEPLSNWDRIRAAVVECEKRKWADLPPVKKNFYIESKATSCMSEMQVINWRKENFNITCDDLKSGEKRLIPKPTCRFKDAFQQYPDLLKSIIRVGIVKPTPIQSQAWPIILQGIDLIVVAQTGTGKTLSYLMPGFIHLDSQPISREQRNGPGMLVLTPTRELALHVEAECSKYSYKGLKSICIYGGRNRNGQIEDISKGVDIIIATPGRLNDLQMNNSVNLRSITYLVIDEADKMLDMEFEPQIRKILLDVRPDRQTVMTSATWPDTVRQLALSYLKDPMIVYVGNLNLVAVNTVKQNIIVTTEKEKRALTQEFVENMSPNDKVIMFVSQKHIADDLSSDFNIQGISAESLHGNSEQSDQERAVEDFKSGNIKILITTDIVSRGLDLNDVTHVYNYDFPRNIDVYVHRVGYIGRTGKTGTSVTLITQRDSKMAGELIKILDRANQSVPEDLVVMAEQYKLNQQKRHRETRSRKPGQRRKEFYFLS

SEQ ID NO: 24 - protein, 648 aa, Homo sapiens
MSHHGGAPKASTWVVASRRSSTVSRAPERRPAEELNRTGPEGYSVGRGGRWRGTSRPPEAVAAGHEELPLCFALKSHFVGAVIGRGGSKIKNIQSTTNTTIQIIQEQPESLVKIFGSKAMQTKAKAVIDNFVKKLEENYNSECGIDTAFQPSVGKDGSTDNNVVAGDRPLIDWDQIREEGLKWQKTKWADLPPIKKNFYKESTATSAMSKVEADSWRKENFNITWDDLKDGEKRPIPNPTCTFDDAFQCYPEVMENIKKAGFQKPTPIQSQAWPIVLQGIDLIGVAQTGTGKTLCYLMPGFIHLVLQPSLKGQRNRPGMLVLTPTRELALQVEGECCKYSYKGLRSVCVYGGGNRDEQIEELKKGVDIIIATPGRLNDLQMSNFVNLKNITYLVLDEADKMLDMGFEPQIMKILLDVRPDRQTVMTSATWPHSVHRLAQSYLKEPMIVYVGTLDLVAVSSVKQNIIVTTEEEKWSHMQTFLQSMSSTDKVIVFVSRKAVADHLSSDLILGNISVESLHGDREQRDREKALENFKTGKVRILIATDLASRGLDVHDVTHVYNFDFPRNIEEYVHRIGRTGRAGRTGVSITTLTRNDWRVASELINILERANQSIPEELVSMAERFKAHQQKREMERKMERPQGRPKKFH

SEQ ID NO: 25 - protein, 139 aa, Homo sapiens
MSWRGRSTYYWPRPRRYVQPPEMIGPMRPEQFSDEVEPATPEEGEPATQRQDPAAAQEGEDEGASAGQGPKPEADSQEQGHPQTGCECEDGPDGQEMDPPNPEEVKTPEEEMRSHYVAQTGILWLLMNNCFLNLSPRKP

SEQ ID NO: 26 - protein, 369 aa, Homo sapiens
MPRAPKRQRCMPEEDLQSQSETQGLEGAQAPLAVEEDASSSTSTSSSFPSSFPSSSSSSSSSCYPLIPSTPEEVSADDETPNPPQSAQIACSSPSVVASLPLDQSDEGSSSQKEESPSTLQVLPDSESLPRSEIDEKVTDLVQFLLFKYQMKEPITKAEILESVIRNYEDHFPLLFSEASECMLLVFGIDVKEVDPTGHSFVLVTSLGLTYDGMLSDVQSMPKTGILILILSIVFIEGYCTPEEVIWEALNMMGLYDGMEHLIYGEPRKLLTQDWVQENYLEYRQVPGSDPARYEFLWGPRAHAEIRKMSLLKFLAKVNGSDPRSFPLWYEEALKDEEERAQDRIATTDDTTAMASASSSATGSFSYPE

SEQ ID NO: 27 - protein, 126 aa, Homo sapiens
MSVMDLANTCSSFQSDLDFCSDCGSVLPLPGAQDTVTCIRCGFNINVRDFEGKVVKTSVVFHQLGTAMPMSVEEGPECQGPVVDRRCPRCGHEGMAYHTRQMRSADEGQTVFYTCTNCKFQEKEDS

SEQ ID NO: 28 - protein, 448 aa, Homo sapiens
MLWLALGPFPAMENQVLVIRIKIPNSGAVDWTVHSGPQLLFRDVLDVIGQVLPEATTTAFEYEDEDGDRITVRSDEEMKAMLSYYYSTVMEQQVNGQLIEPLQIFPRACKPPGERNIHGLKVNTRAGPSQHSSPAVSDSLPSNSLKKSSAELKKILANGQMNEQDIRYRDTLGHGNGGTVYKAYHVPSGKILAVKVILLDITLELQKQIMSELEILYKCDSSYIIGFYGAFFVENRISICTEFMDGGSLDVYRKMPEHVLGRIAVAVVKGLTYLWSLKILHRDVKPSNMLVNTRGQVKLCDFGVSTQLVNSIAKTYVGTNAYMAPERISGEQYGIHSDVWSLGISFMELALGRFPYPQIQKNQGSLMPLQLLQCIVDEDSPVLPVGEFSEPFVHFITQCMRKQPKERPAPEELMGHPFIVQFNDGNAAVVSMWVCRALEERRSQQGPP

SEQ ID NO: 29 - protein, 317 aa, Homo sapiens
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 30 - protein, 712 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEV

SEQ ID NO: 31 - protein, 288 aa, Homo sapiens
MSATGDRHPTQGDQEAPVSQEGAQAEAAGAGNQEGGDSGPDSSDVVPAAEVVGVAGPVEGLGEEEGEQAAGLAAVPRGGSAEEDSDIGPATEEEEEEEGNEAANFDLAVVARRYPASGIHFVLLDMVHSLLHRLSHNDHILIENRQLSRLMVGPHAAARNLWGNLPPLLLPQRLGAGAAARAGEGLGLIQEAASVPEPAVPADLAEMAREPAEEAAEEKLSEEATEEPDAEEPATEEPTAQEATAPEEVTKSQPEKWDEEAQDAAGEEEKEQEKEKDAENKVKNSKGT

SEQ ID NO: 32 - protein, 579 aa, Homo sapiens
MNKLYIGNLSENAAPSDLESIFKDAKIPVSGPFLVKTGYAFVDCPDESWALKAIEALSGKIELHGKPIEVEHSVPKRQRIRKLQIRNIPPHLQWEVLDSLLVQYGVVESCEQVNTDSETAVVNVTYSSKDQARQALDKLNGFQLENFTLKVAYIPDEMAAQQNPLQQPRGRRGLGQRGSSRQGSPGSVSKQKPCDLPLRLLVPTQFVGAIIGKEGATIRNITKQTQSKIDVHRKENAGAAEKSITILSTPEGTSAACKSILEIMHKEAQDIKFTEEIPLKILAHNNFVGRLIGKEGRNLKKIEQDTDTKITISPLQELTLYNPERTITVKGNVETCAKAEEEIMKKIRESYENDIASMNLQAHLIPGLNLNALGLFPPTSGMPPPTSGPPSAMTPPYPQFEQSETETVHLFIPALSVGAIIGKQGQHIKQLSRFAGASIKIAPAEAPDAKVRMVIITGPPEAQFKAQGRIYGKIKEENFVSPKEEVKLEAHIRVPSFAAGRVIGKGGKTVNELQNLSSAEVVVPRDQTPDENDQVVVKITGHFYACQVAQRKIQEILTQVKQHQQQKALQSGPPQSRRK

SEQ ID NO: 33 - protein, 210 aa, Homo sapiens
MQAEGQGTGGSTGDADGPGGPGIPDGPGGNAGGPGEAGATGGRGPRGAGAARASGPRGGAPRGPHGGAASAQDGRCPCGARRPDSRLLQLHITMPFSSPMEAELVRRILSRDAAPLPRPGAVLKDFTVSGNLLFMSVRDQDREGAGRMRVVGWGLGSASPEGQKARDLRTPKHKVSEQRPGTPGPPPPEGAQGDGCRGVAFNVMFSAPHI

SEQ ID NO: 34 - protein, 409 aa, Homo sapiens
MQVTLKTLQQQTFKIDIDPEETVKALKEKIESEKGKDAFPVAGQKLIYAGKILNDDTALKEYKIDEKNFVVVMVTKPKAVSTPAPATTQQSAPASTTAVTSSTTTTVAQAPTPVPALAPTSTPASITPASATASSEPAPASAAKQEKPAEKPAETPVATSPTATDSTSGDSSRSNLFEDATSALVTGQSYENMVTEIMSMGYEREQVIAALRASFNNPDRAVEYLLMGIPGDRESQAVVDPPQAASTGAPQSSAVAAAAATTTATTTTTSSGGHPLEFLRNQPQFQQMRQIIQQNPSLLPALLQQIGRENPQLLQQISQHQEHFIQMLNEPVQEAGGQGGGGGGGSGGIAEAGSGHMNYIQVTPQEKEAIERLKALGFPEGLVIQAYFACEKNENLAANFLLQQNFDED

SEQ ID NO: 35 - protein, 208 aa, Homo sapiens
MDPFLVLLHSVSSSLSSSELTELKFLCLGRVGKRKLERVQSGLDLFSMLLEQNDLEPGHTELLRELLASLRRHDLLRRVDDFEAGAAAGAAPGEEDLCAAFNVICDNVGKDWRRLARQLKVSDTKIDSIEDRYPRNLTERVRESLRIWKNTEKENATVAHLVGALRSCQMNLVADLVQEVQQARDLQNRSGAMSPMSWNSDASTSEAS

SEQ ID NO: 36 - protein, 420 aa, Homo sapiens
MSSPRDFRAEPVNDYEGNDSEAEDLNFRETLPSSSQENTPRSKVFENKVNSEKVKLSLRNFPHNDYEDVFEEPSESGSDPSMWTARGPFRRDRWSSEDEEAAGPSQALSPLLSDTRKIVSEGELDQLAQIRPLIFNFHEQTAIKDCLKILEEKTAAYDIMQEFMALELKNLPGEFNSGNQPSNREKNRYRDILPYDSTRVPLGKSKDYINASYIRIVNCGEEYFYIATQGPLLSTIDDFWQMVLENNSNVIAMITREIEGGIIKCYHYWPISLKKPLELKHFRVFLENYQILQYFIIRMFQVVEKSTGTSHSVKQLQFTKWPDHGTPASADSFIKYIRYARKSHLTGPMVVHCSAGIGRTGVFLCVDVVFCAIVKNCSFNIMDIVAQMREQRSGMVQTKEQYHFCYDIVLEVLRKLLTLD

SEQ ID NO: 37 - protein, 284 aa, Homo sapiens
MDAIKKKMQMLKLDKENALDRAEQAEADKKAAEDRSKQLEDELVSLQKKLKGTEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDRYEEEIKVLSDKLKEAETRAEFAERSVTKLEKSIDDLEDELYAQKLKYKAISEELDHALNDMTSI

SEQ ID NO: 38 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

(19) United States
(12) Patent Application Publication
(10) Pub. No.: US 2026/0287588 A1
(43) Pub. Date: Sep. 24, 2026

(54) Detection Of Biomarkers For Non-Small Cell Lung Cancer

(71) Applicants: SENGENICS SDN BHD, Kuala Lumpur (MY); NATIONAL UNIVERSITY OF SINGAPORE, Singapore (SG)

(72) Inventors: Jonathan Michael BLACKBURN, Singapore (SG); Arif ANWAR, Kuala Lumpur (MY); Boon Cher GOH, Singapore (SG); Lingzhi WANG, Singapore (SG); Sok Hwee Esther CHEOW, Singapore (SG); Ross Andrew SOO, Singapore (SG); Win Lwin THUYA, Singapore (SG)

(73) Assignees: SENGENICS SDN BHD, Kuala Lumpur (MY); NATIONAL UNIVERSITY OF SINGAPORE, Singapore (SG)

(21) Appl. No.: 19/636,049

(22) Filed: Apr. 1, 2026

Related U.S. Application Data
(62) Division of application No. 17/413,474, filed on Dec. 12, 2019, now Pat. No. 12,601,741.

(30) Foreign Application Priority Data
Dec. 12, 2018 (SG) 10201811119X

(51) Int. Cl. G01N 33/5752; G01N 33/543; G01N 33/68

(52) U.S. Cl. CPC G01N 33/5752; G01N 33/54306; G01N 33/6854

(57) ABSTRACT

A method for diagnosing Non-Small Cell Lung Cancer (NSCLC) from a sample extracted from a subject by testing the sample for the presence of biomarkers, the biomarkers being autoantibodies against XAGE1D, LRRFIP2, and GAGE2C. Also claimed are a method of manufacturing a kit, and compositions comprising a panel of said antigens or exosomal autoantibodies.

### CROSS-REFERENCE TO RELATED APPLICATIONS

This application is a divisional of U.S. patent application Ser. No. 17/413,474 filed on Jun. 11, 2021 which is a U.S. National Phase Application under 35 U.S.C. 371 of International Application No. PCT/SG2019/050611 filed on Dec. 12, 2019, which claims the benefit of priority from Singapore Application No. 10201811119X filed Dec. 12, 2018. The entire disclosures of each of the above applications are incorporated herein by reference.

### REFERENCE TO AN ELECTRONIC SEQUENCE LISTING

This application contains references to nucleic acid sequences and/or amino acid sequences which have been submitted concurrently herewith as the sequence listing XML file entitled “16742S-1-US-DVA_Sequence_Listing.xml”, file size 58,928 Bytes (B), created on Mar. 30, 2026. The aforementioned sequence listing is hereby incorporated by reference in its entirety.

### FIELD

The invention relates to the detection of biomarkers for Non-Small Cell Lung Cancer (NSCLC).

### BACKGROUND

Despite technological advances in the area of proteomics research, there are only a handful of biomarkers that have entered the clinic, and 90% of the biomarkers are protein biomarkers. Autoantibody biomarkers as described herein are autoantibodies to antigens, autoantibodies being antibodies which are produced by an individual which are directed against one or more of the individual's own proteins (‘self’ antigens). Some of the main reasons for failure of biomarkers to make it into clinical practice are:
1) Low sensitivity and specificity of diagnosis of cancerous diseases
2) Low prognostic/predictive value
3) Not important for clinical decision making
4) Original claims fail validation (false discoveries)

For Non-small cell Lung Cancer (NSCLC), although many individual proteins have been reported to aid diagnosis and prognosis, very few have demonstrated sufficient value to be introduced into clinical use. Furthermore, many protein biomarkers discovered in the serum/plasma samples seem to overlap with other diseases, especially other cancers and inflammatory diseases.

An aim of the invention therefore is to provide an improved panel of autoantibody biomarkers for the detection of Non-Small Cell Lung Cancer.

### SUMMARY OF INVENTION

In one aspect of the invention, there is provided a method for diagnosing Non-Small Cell Lung Cancer from a sample extracted from a subject, comprising the steps of:
(i) testing the sample for the presence of autoantibody biomarkers specific for Non-Small Cell Lung Cancer;
(ii) determining whether the subject has Non-Small Cell Lung Cancer based on the detection of said autoantibody biomarkers;
characterised in that the biomarkers are autoantibodies to antigens comprising XAGE1D, LRRFIP2 and GAGE2C.

Advantageously the autoantibody biomarkers can be used in the diagnosis of non-small cell lung cancer.

In one embodiment the sample is tested using a panel of antigens that correspond to the autoantibody biomarkers. Typically the antigens are biotinylated proteins. Advantageously the biotinylation ensures that the antigens are folded in their correct form to ensure accuracy of detection by the autoantibody biomarkers.

In one embodiment the antigens further comprise one or more of DDX53, DDX43, GAGE1, MAGEA10, ZNRD1, MAP2K5, MAGEA4, STAT1, CT47A1, IGF2BP3, CTAG2, RAD23B, FADD, PTPN20A, TPM1, CTAG1A.

It should be noted that not all antigens generate an autoantibody response and it is not possible to predict a priori which antigens will do so in a given cancer patient cohort—of more than 1600 antigens tested, only autoantibodies against the 19 antigens described above are suitable as biomarkers in NSCLC. Advantageously some of the 19 antigens are recognised by autoantibody biomarkers even when the well-known EGFR test for NSCLC is negative.

In one embodiment each biotinylated protein is formed from a Biotin Carboxyl Carrier Protein (BCCP) folding marker which is fused in-frame with the protein.

In one embodiment the biotinylated proteins are bound to a streptavidin-coated substrate.

Advantageously full-length proteins are expressed as fusions to the BCCP folding marker which itself becomes biotinylated in vivo when the fusion partner is correctly folded. By comparison misfolded fusion partners cause the BCCP to remain in the ‘apo’ (i.e. non-biotinylated) form such that it cannot attach to a streptavidin substrate. Thus only correctly folded fusion proteins become attached to the streptavidin substrate via the biotin moiety appended to the BCCP tag.

In one embodiment the substrate comprises a glass slide, biochip, strip, slide, bead, microtitre plate well, surface plasmon resonance support, microfluidic device, thin film polymer base layer, hydrogel-forming polymer base layer, or any other device or technology suitable for detection of antibody-antigen binding.

In one embodiment the substrate is exposed to a sample extracted from a person, such that autoantibody biomarkers from the sample may bind to the antigens.

Typically the sample comprises any or any combination of exosomes, blood, serum, plasma, urine, saliva, amniotic fluid, cerebrospinal fluid, breast milk, semen or bile.

Advantageously as exosomes contain membrane-bound proteins that reflect their originating cell, and in cancer have been shown to be implicated in the crosstalk between tumour cells and normal cells thereby facilitating the malignant process, exosomes have been found to be promising as enriched sources of diagnostic and prognostic markers. The exosomal autoantibody biomarkers detected using the BCCP folding marker technology are therefore potentially superior compared to the majority of serological biomarkers identified using conventional approaches.

In one embodiment following exposure to the sample, the substrate is exposed to a fluorescently-tagged secondary antibody to allow the amount of any autoantibodies from the sample bound to the antigens on the panel to be determined. Typically the secondary antibody is anti-human IgG, but it will be appreciated that other secondary antibodies could be used, such as anti-IgM, anti-IgG1, anti-IgG2, anti-IgG3, anti-IgG4 or anti-IgA.

In one embodiment the presence of non-small cell lung cancer corresponds to the relative or absolute amount of autoantibodies from the sample specifically binding to the antigens.

In one embodiment the method is performed in vitro.

In a further aspect of the invention, there is provided a method for manufacturing a kit for diagnosing Non-Small Cell Lung Cancer from a sample extracted from a subject, comprising the steps of:
for each antigen in a panel, cloning a biotin carboxyl carrier protein folding marker in-frame with a gene encoding the antigen and expressing the resulting biotinylated antigen;
binding the biotinylated antigens to addressable locations on one or more streptavidin-coated substrates, thereby forming an antigen array;
such that the amount of autoantibodies from the sample binding to the antigens on the panel can be determined by exposing the substrate to the sample and measuring the response;
characterised in that the antigens comprise XAGE1D, LRRFIP2 and GAGE2C.

In one embodiment the antigens further comprise one or more of DDX53, DDX43, GAGE1, MAGEA10, ZNRD1, MAP2K5, MAGEA4, STAT1, CT47A1, IGF2BP3, CTAG2, RAD23B, FADD, PTPN20A, TPM1, CTAG1A.

In one embodiment the method comprises detecting upregulation/downregulation of one or more autoantibody biomarkers. Thus the method can be used for monitoring the response of a subject undergoing chemo/targeted/immuno-therapy for lung cancer and stratifying the subjects based on their autoantibody profile.

In a further aspect of the invention there is provided a method for detecting non-small cell lung cancer by exposing a composition comprising a panel of antigens as herein described to a sample extracted from a person, and determining the level of autoantibodies from the sample binding to the antigens.

In a yet further aspect of the invention there is provided a method for diagnosing non-small cell lung cancer by exposing a composition comprising a panel of antigens as herein described to a sample extracted from a person in vitro, and determining the level of autoantibodies from the sample binding to the antigens.

In further aspect of the invention, there is provided a composition comprising a panel of antigens for detecting non-small cell lung cancer, characterised in that the antigens comprise XAGE1D, LRRFIP2 and GAGE2C

In one embodiment the antigens further comprise one or more of DDX53, DDX43, GAGE1, MAGEA10, ZNRD1, MAP2K5, MAGEA4, STAT1, CT47A1, IGF2BP3, CTAG2, RAD23B, FADD, PTPN20A, TPM1, CTAG1A.

In one embodiment the antigens are biotinylated proteins.

In one embodiment the amount of one or more exosomal autoantibody biomarkers binding in vitro to the antigens in a sample from a patient can be measured to determine the presence of non-small cell lung cancer.

In yet further aspect of the invention, there is provided a composition comprising a panel of exosomal autoantibody biomarkers for detecting non-small cell lung cancer;
wherein the levels of exosomal autoantibody biomarkers are measured in a sample from a NSCLC patient;
characterised in that the exosomal autoantibody biomarkers are selected from autoantibodies specific for at least X Antigen Family Member 1D (XAGE1D), LRR Binding FLII Interacting Protein 2 (LRRFIP2) and G Antigen 2C (GAGE2C).

### BRIEF DESCRIPTION OF THE DRAWINGS

It will be convenient to further describe the present invention with respect to the accompanying drawings that illustrate possible arrangements of the invention. Other arrangements of the invention are possible, and consequently the particularity of the accompanying drawings is not to be understood as superseding the generality of the preceding description of the invention.

FIG. 1 illustrates the structure of the E. coli Biotin Carboxyl Carrier Protein domain.

FIG. 2 is a graph illustrating variable importance scores across 19 biomarkers identified from the NSCLC study.

FIG. 3 illustrates ROC curves for 19 biomarkers.

FIG. 4 is a graph illustrating levels of the autoantibody biomarkers at different stages of NSCLC.

FIG. 5 illustrates a comparison of the levels of the core biomarker panel (XAGE1D, LRRFIP2 and GAGE2C) with respect to EGFR status in patients.

FIG. 6 illustrates the overall autoantibody profiles of the core set of biomarkers in three different stages of NSCLC.

FIG. 7 illustrates the distinct molecular signatures for the 19 shortlisted biomarkers for NSCLC patients.

FIG. 8 illustrates the pPRO9 plasmid used as a vector.

FIG. 9 illustrates the ROC curve for the 19 shortlisted biomarkers for NSCLC (XAGE1D, DDX53, GAGE2C, LRRFIP2, GAGE1, DDX43, MAGEA10, ZNRD1, STAT1, MAP2K5, MAGEA4, IGF2BP3, FADD, RAD23B, CT47A1, CTAG2, PTPN20A, TPM1, CTAG1A).

FIG. 10 illustrates the ROC curves for the best panel of 7 biomarkers in the validation study.

FIG. 11 illustrates the ROC curves for the 19 biomarkers in the validation study.

### DETAILED DESCRIPTION

### Materials and Methods

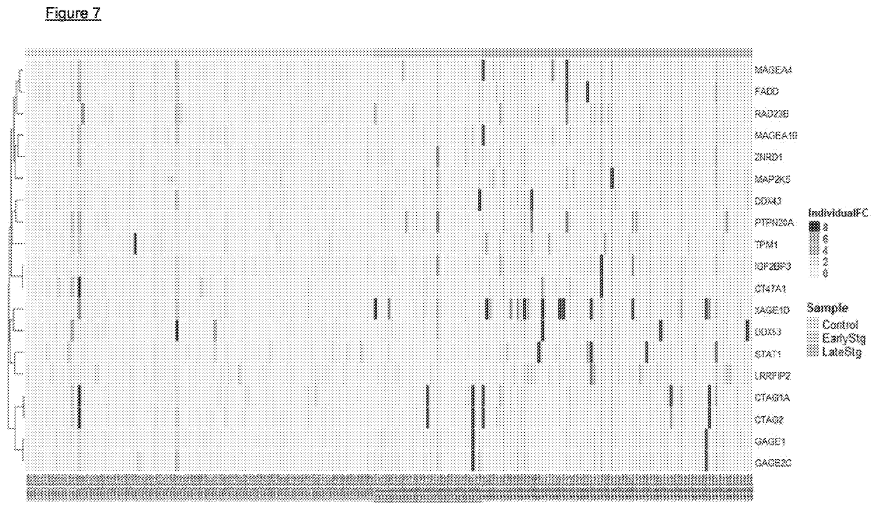

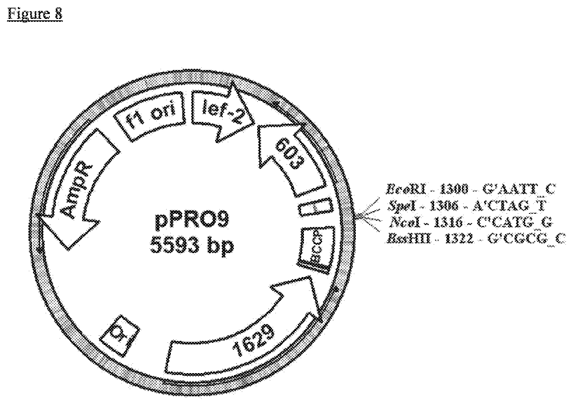

Gene synthesis and cloning. The pPRO9 plasmid (see FIG. 8 below) was constructed by standard techniques and consists of a c-myc tag and BCCP protein domain, preceded by a multi-cloning site. A synthetic gene insert was assembled from synthetic oligonucleotides and/or PCR products. The fragment was cloned into pPRO9 using SpeI and NcoI cloning sites. The plasmid DNA was purified from transformed bacteria and concentration determined by UV spectroscopy. The final construct was verified by sequencing. The sequence congruence within the used restriction sites was 100%. 5 μg of the plasmid preparation was lyophilized for storage.

The recombinant baculoviruses are generated via co-transfection of a bacmid carrying the strong viral polyhedrin promoter together with a transfer vector carrying the coding sequences of protein of interest, into the Sf9 cell line which is a clonal isolate derived from the parental Spodoptera frugiperda cell line IPLB-Sf-21-AE. Homologous recombination initiated by the viral system causes the transfected cells to show signs of viral cytopathic effect (CPE) within few days of culture incubation. The most common CPE observed was the significantly enlargement of average cell size, a consequences of viral progeny propagation. These baculoviruses known as P0 were then released into the culture medium, and viral amplification were done to generate a higher titre of P1 viruses.

Protein Expression. Expressions were carried out in 24 well blocks using 3 ml cultures containing 6×10⁶ Sf9 cells per well. High titre, low passage, viral stocks of recombinant baculovirus (>10⁷ pfu/ml) were used to infect sf9 insect cells. The infected cells were then cultured for 72 hours to allow them to produce the recombinant protein of interest. The cells were washed with PBS, resuspended in buffer, and were frozen in aliquots at −80° C. ready for lysis as required. Depending on the transfer vector construct and the nature of the protein itself, recombinant protein lysate can be pelleted either from the cultured cell or the cultured medium. Positive recombinant proteins were then analyzed via SDS-PAGE and Western blot against Streptavidin-HRP antibody. In total, 1630 human antigens were cloned and expressed using this methodology.

Array fabrication. HS (hydrogel-streptavidin) slides were purchased from Schott and used to print the biotinylated proteins. A total of 9 nanoliters of crude protein lysate was printed on a HS slide in quadruplicate using non-contact piezo printing technology. Print buffer that have a pH between 7.0 and 7.5 were used. The slides were dried by centrifugation (200×g for 5 min) before starting the washing and blocking. The printed arrays were blocked with solutions containing BSA or casein (concentration: 0.1 mg/ml) in a phosphate buffer. The pH was adjusted to be between 7.0 and 7.5 and cold solutions were used (4° C.-20° C.). Slides were not allowed to dry between washes, and were protected from light. In total, each resultant ‘Immunome array’ comprised 1630 antigens, each printed in quadruplicate.

Experimental Procedure. Each critical experimental step of running the Immunome array required a second trained person to thoroughly check, precisely record and cross-check all steps in the protocol, in order to reduce operator bias. Samples were picked, randomised and assigned to assay racks accordingly. These samples were then stored at −20° C. until the experimental setup was complete.

### 1. Study Cohort

A cohort comprising of 209 participants between the age of 29 and 85 was recruited for the study. The subjects were selected across more than 6 ethnicities diagnosed with different types of lung cancer including adenocarcinoma, squamous cell carcinoma, non-small cell lung carcinoma, large cell carcinoma and other types of lung malignancies. A total of 31 patients were diagnosed with early stage lung cancer while 78 patients were diagnosed with late stage lung cancer. A total of 33 subjects were smoker and 44 subjects were non-smoker. EGFR was tested positive in 30 subjects and negative in 39 subjects. A total of 100 samples from age and gender-matched healthy subjects were also collected.

### 2. Sample Preparation

A total of 209 plasma samples were collected from the above cohort and exosomes were isolated from each sample using an Invitrogen Total Exosome Isolation kit (Thermo Fisher cat. no. 4484450). Exosomal preparations were frozen at −20° C. until use.

Exosome samples were placed in a shaking incubator set at +20° C. and allowed to thaw for 30 minutes. When completely thawed, each sample was vortexed vigorously three times at full speed and spun down for 3 minutes at 13,000 rpm using a microcentrifuge. 22.5 μL of the sample was pipetted into 4.5 mL of Serum Assay Buffer (SAB) containing 0.1% v/v Triton, 0.1% w/v BSA in PBS (20° C.) and vortexed to mix three times. The tube was tilted during aspiration to ensure that the plasma was sampled from below the lipid layer at the top but does not touch the bottom of the tube in case of presence of any sediment. This exosome dilution process was carried out in a class II biological safety cabinet. Batch records were marked accordingly to ensure that the correct samples were added to the correct tubes.

Other types of samples, such as serum, plasma, blood, urine, saliva, amniotic fluid, cerebrospinal fluid, breast milk, semen or bile were diluted as per the above protocol before assay.

### 3. Biomarker Assay

Each protein microarray was removed from the storage buffer using forceps, placed in the slide box and rack containing 200 mL cold SAB and shaken on an orbital shaker at 50 rpm, for 5 minutes. After washing, each protein microarray was placed, array side up, in a slide hybridization chamber with individual plasma which had been diluted earlier. All slides were scanned using the barcode scanner into the relevant batch record and incubated on a horizontal shaker at 50 rpm for 2 hours at 20° C.

4. Array Washing after Plasma Binding

The protein microarray slide was then rinsed twice in individual “Pap jars” with 30 mL SAB, followed by 200 mL of SAB buffer in the slide staining box for 20 minutes on the shaker at 50 rpm at room temperature. All slides were transferred sequentially and in the same orientation.

5. Incubation with Cy3-Anti IgG

Binding of autoantibodies to the arrayed antigens on replica Immunome arrays was detected by incubation with Cy3-rabbit anti-human IgG (Dako Cytomation) labelled according to the manufacturer's recommended protocols (GE Healthcare). Arrays were immersed in hybridization solution containing a mixture of Cy3-rabbit antihuman IgG solution (diluted 1:1000 in SAB buffer) and shaken for 2 hours at 50 rpm at 20° C.

6. Washing after Incubation with Cy3-Anti IgG

After incubation, each slide was washed in 200 mL of SAB buffer, 3 times for 5 minutes, with shaking at 50 rpm at room temperature. Excess buffer was removed by immersing the slide in 200 mL of pure water for a few minutes. Slides were then dried for 2 min at 240 g at room temperature. Slides were then stored at room temperature until scanning (preferably the same day). Hybridization signals were measured with a microarray laser scanner (Agilent Scanner) at 10 μm resolution. Fluorescence intensities were detected according to the manufacturer's instructions, whereby each spot is plotted using Agilent Feature Extraction software.

Spot segmentation Semi-automatic QC process was carried out in order to produce a viable result. The output from the microarray scanner is a raw tiff format image file. Extraction and quantification of each spot on the array were performed using the GenePix Pro 7 software (Molecular Devices). A GAL (GenePix Array List) file for the array was generated to aid with image analysis. GenePix Pro 7 allows for automatic spot gridding and alignment of each spot on the array for data extraction. Following data extraction, a GenePix Results (.GPR) file was generated for each slide which contains numerical information for each spot; Protein ID, protein name, foreground intensities, background intensities etc.

### Bioinformatics Analysis.

### Image Analysis: Raw Data Extraction

The aim of an image analysis is to evaluate the amount of autoantibody present in the plasma sample by measuring the median intensities of all the pixels within each probed spot. A raw tiff format image file is generated for each slide, i.e. each sample. Automatic extraction and quantification of each spot on the array are performed using the GenePix Pro 7 software (Molecular Devices) which outputs the statistics for each probed spot on the array. This includes the mean and median of the pixel intensities within a spot along with its local background. A GAL (GenePix Array List) file for the array is generated to aid with image analysis. This file contains the information of all probed spots and their positions on the array. Following data extraction, a GenePix Results (.GPR) file is generated for each slide which contains the information for each spot; Protein ID, protein name, foreground intensities, background intensities etc. In the data sheet generated from the experiment, both foreground and background intensities of each spot are represented in relative fluorescence units (RFUs).

### Data Handling and Pre-Processing

For each slide, proteins and control probes are spotted in quadruplicate—4 arrays on each slide. The following steps were performed to verify the quality of the protein array data before proceeding with data analysis:

### Step 1:

Calculate net intensities for each spot by subtracting background signal intensities from the foreground signal intensities of each spot. For each spot, the background signal intensity was calculated using a circular region with three times the diameter of the spot, centered on the spot.

### Step 2:

Remove replica spots with RFU 0.

### Step 3:

Zero net intensities if only 1 replica spot remaining.

### Step 4:

Calculating percentage of coefficient of variant (CV %) of to determine the variations between the replica spots on each slide.

$$CV \% = \frac{S . D .}{Mean} \times 100 \% \qquad Equation 1$$

Flag a set of replica spots with only 2 or less replica/s remaining and CV %>20% as “High CV”. The mean RFU of these replica spots (i.e. proteins) will be excluded from the downstream analysis.

For proteins/controls with a CV %>20% and with 3 or more replica spots remaining, the replica spots which result in this high CV % value were filtered out. This was done by calculating the standard deviation between the median value of the net intensities and individual net intensities for each set of replica spots. The spot with the highest standard deviation was removed. CV % values were re-calculated and the process repeated.

### Step 5:

Calculating the mean of the net intensities for the remaining replica spots.

### Step 6:

Inspecting signal intensities of two positive controls: IgG and Cy3-BSA.

### Step 7:

Composite normalisation of data using both quantile-based and total intensity-based modules. This method assumes that different samples share a common underlying distribution of their control probes while taking into account the potential existence of flagged spots within them. The Immunome array uses Cy3-labelled biotinylated BSA (Cy3-BSA) replicates as the positive control spots across slides. Hence it is considered as a housekeeping probe for normalisation of signal intensities for any given study.

The quantile module adopts the algorithm described by Bolstad et al., 2003. This reorganisation enables the detection and handling of outliers or flagged spots in any of the Cy3BSA control probes. A total intensity-based module was then implemented to obtain a scaling factor for each sample. This method assumes that post-normalisation, the positive controls should have a common total intensity value across all samples. This composite method aims to normalise the protein array data from variations in their measurements whilst preserving the targeted biological activity across samples. The steps are as follows:

### Quantile-Based Normalisation of all Cy3BSA Across all Samples

(i=spot number and j=sample number)
1. Load all Cy3-BSA across all samples, j, into an i X j matrix X
2. Sort spot intensities in each column j of X to get Xₛₒᵣₜ
3. Take the mean across each row i of Xₛₒᵣₜ to get <Xᵢ>

### Intensity-Based Normalisation

1. Calculate sum of the mean across each row i, Σ<Xi>
2. For each sample, k, calculate the sum of all Cy3-BSA controls, ΣXk
3. For each sample, k,

$$Scaling factor ( k ) = \frac{\sum < Xi >}{\sum Xk} \qquad Equation 2$$

### Data Analysis

High concentrations of an arrayed protein may occasionally give a “false” positive signal in serology assays because of concentration-driven, non-selective binding of an immunoglobulin to the target. This can arise theoretically due to an avidity effect: weak, non-specific immunoglobulin binding sites on a specific protein becoming coupled across multiple neighbouring protein molecules via an antibody as a result of the high density of immobilized protein, thus making the protein appear to be highly antigenic. Whenever this phenomenon occurs, it would be expected to be observed in the healthy control samples and will give rise to high intensity signals and/or signals that are close to saturation on the arrays. In Sengenics Immunome, proteins such as RBPJ and IGHG1 show consistently high signal intensities across all samples.

For this reason, given a large sample number (i.e. 100-200 samples) and availability of sample cohort, a penetrance-based fold change (pFC) analysis method is implemented for the identification of highly expressed proteins in each case sample. This method will remove any false positive signals from the data by setting a protein-specific threshold (i.e. background threshold). This defined per-protein background threshold is calculated based on the signal intensities for each specific protein measured for a given cohort of healthy control samples. A step-by-step description of this method is as follows:

### Step 1:

Individual fold changes for both case and control are calculated by dividing the RFU value for each protein in each sample, H, by the mean of the RFU values of each protein across all the control samples (i.e. background threshold).

$$Individual FC = \frac{{H}_{Case or Control}}{μ ( {H}_{Control} )} \qquad Equation 3$$

### Step 2:

For proteins with individual fold change of less than 2 fold above the background threshold, their signal intensities (RFU) are replaced with zeroes.

### Step 3:

Penetrance frequency (number of case and control samples with individual fold changes≥2 fold) for both case (Frequency_{Case}) and control (Frequency_{Control}) are determined for each protein along with their difference.

$${Frequency}_{Case} = n ( Individual FC ( Case ) ) \geq 2 ) \qquad Equation 4$$

$${Frequency}_{Control} = n ( Individual FC ( Control ) ) \geq 2 ) \qquad Equation 5$$

$${Frequency}_{diff} = {Frequency}_{Case} - {Frequency}_{Control} \qquad Equation 6$$

### Step 4:

Penetrance Fold Changes for both case and control groups are calculated for each protein.

$$Penetrance Fold {Change}_{case} = \frac{μ ( {H}_{Case} [ i ] )}{μ ( {H}_{Control} )}$$

$$Penetrance Fold {Change}_{control} = \frac{μ ( {H}_{Control} [ i ] )}{μ ( {H}_{Control} )}$$

$${H}_{Case} [ i ] = {H}_{Case} with FC Case \geq 2 fold$$

$${H}_{Control} [ i ] = {H}_{Control} with FC Control \geq 2 fold$$

Putative biomarkers are identified and ranked according to the following criteria:

$$Penetrance Fold {Change}_{case} \geq 2.$$

$$\% {Frequency}_{Case} \geq 10 \%$$

$$\% Frequency differential \geq 10 \%$$

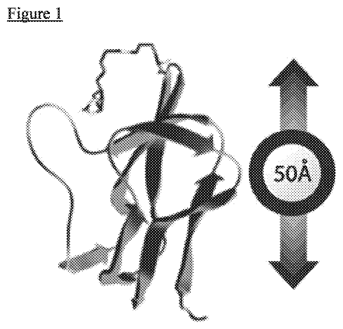

The invention utilises the Biotin Carboxyl Carrier Protein (BCCP) folding marker which is cloned in-frame with the gene encoding the protein of interest, as described above and in EP1470229. The structure of the E. coli BCCP domain is illustrated in FIG. 1, wherein residues 77-156 are drawn (coordinate file 1bdo) showing the N- and C-termini and the single biotin moiety that is attached to lysine 122 in vivo by biotin ligase.

BCCP acts not only as a protein folding marker but also as a protein solubility enhancer. BCCP can be fused to either the N- or C-terminal of a protein of interest. Full-length proteins are expressed as fusions to the BCCP folding marker which becomes biotinylated in vivo, but only when the protein is correctly folded. Conversely, misfolded proteins drive the misfolding of BCCP such that it is unable to become biotinylated by host biotin ligases. Hence, misfolded proteins are unable to specifically attach to a streptavidin-coated solid support. Therefore only correctly folded proteins become attached to a solid support via the ECCP tag.

The surface chemistry of the support is designed carefully and may use a three-dimensional thin film polymer base layer (polyethylene glycol, PEG), which retains protein spot morphologies and ensures consistent spot sizes across the array. The PEG layer inhibits non-specific binding, therefore reducing the high background observed using other platforms. The solid support used to immobilize the selected biomarkers is thus designed to resist non-specific macromolecule adsorption and give excellent signal-to-noise ratios and low limits of detection (i.e. improved sensitivity) by minimising non-specific background binding. In addition the PEG layer also preserves the folded structure and functionality of arrayed proteins and protein complexes post-immobilisation. This is critical for the accurate diagnosis because human serum antibodies are known in general to bind non-specifically to exposed hydrophobic surfaces on unfolded proteins, thus giving rise to false positives in serological assays on arrays of unfolded proteins, moreover, human autoantibodies typically bind to discontinuous epitopes, so serological assays on arrays of unfolded proteins or mis-folded proteins will also give rise to false negatives in autoantibody binding assays.

As biotinylated proteins bound to a streptavidin-coated surface show negligible dissociation, this interaction therefore provides a superior means for tethering proteins to a planar surface and is ideal for applications such as protein arrays, SPR and bead-based assays. The use of a compact, folded, biotinylated, 80 residue domain BCCP affords two significant advantages over for example the AviTag and intein-based tag. First, the BCCP domain is cross-recognised by eukaryotic biotin ligases enabling it to be biotinylated efficiently in yeast, insect, and mammalian cells without the need to co-express the E. coli biotin ligase. Second, the N- and C-termini of BCCP are physically separated from the site of biotinylation by 50 Å (as shown in FIG. 1), so the BCCP domain can be thought of as a stalk which presents the recombinant proteins away from the solid support surface, thus minimising any deleterious effects due to immobilisation.

The success rate of BCCP folding marker mediated expression of even the most complex proteins is in excess of 98%. The technology can therefore be applied in a highly parallelised pipeline resulting in high-throughput, highly consistent production of functionally validated proteins.

The addition of BCCP permits the monitoring of fusion protein folding by measuring the extent of in vivo biotinylation. This can be measured by standard blotting procedures, using SDS-PAGE or in situ colony lysis and transfer of samples to a membrane, followed by detection of biotinylated proteins using a streptavidin conjugate such as streptavidin-horseradish peroxidase. Additionally, the fact that the BCCP domain is biotinylated in vivo is particularly useful when multiplexing protein purification for fabrication of protein arrays since the proteins can be simultaneously purified from cellular lysates and immobilised in a single step via the high affinity and specificity exhibited by a streptavidin surface.

The biomarkers of the present invention can be used in early diagnosis of NSCLC, patient stratification and treatment monitoring. This includes any distinguishable manifestation of the condition, including not having NSCLC. The test can determine the presence or absence of NSCLC in a patient, the risk of developing NSCLC, the stage or severity of NSCLC and the effectiveness or response to treatment of NSCLC. Based on this status, further medical procedures may be indicated, including additional diagnostic tests or therapeutic procedures or regimens.

The microarray prototype and final product can be multiplexed far beyond the technical capability of other immunoassay systems and will enable exquisitely sensitive and specific testing of patients and high-risk population for NSCLC.

The power of a diagnostic test to correctly predict status is commonly measured as the sensitivity and specificity of the assay or the area under a receiver operated characteristic (“ROC”) curve. Sensitivity is the percentage of true positives that are correctly predicted to be positive, while specificity is the percentage of true negatives that are correctly predicted to be negative. The greater the area under the ROC curve, the higher the prediction power of the test.

Autoantibody biomarkers were determined by Penetrance fold change method where age matched controls are considered as baseline to observe the elevated frequencies (2 Foldchange) of individual biomarkers in lung cancer patients. The list of the biomarkers identified here for diagnosis of NSCLC is shown in Table 1.

TABLE 1

| | | Penetrance | | Penetrance | | Penetrance |
|---|---|---|---|---|---|---|
| | Penetrance | Frequency | Mean | Fold | Penetrance | Frequency |
| | Frequency | % | Penetrance | Change | Frequency | % |
| Protein | (NSCLC) | (NSCLC) | (NSCLC) | (NSCLC) | (Control) | (Control) |
| XAGE1D | 26 | 23.85321 | 24654.94 | 9.678011 | 4 | 4 |
| PTPN20A | 17 | 15.59633 | 12505.05 | 3.883074 | 4 | 4 |
| TPM1 | 15 | 13.76147 | 16940.47 | 2.845931 | 6 | 6 |
| CTAG1A | 14 | 12.84404 | 23341.53 | 10.43956 | 4 | 4 |
| RAD23B | 14 | 12.84404 | 8193.595 | 3.231442 | 5 | 5 |
| ZNRD1 | 14 | 12.84404 | 12647.16 | 2.988961 | 4 | 4 |
| CTAG2 | 13 | 11.92661 | 20739.05 | 8.484654 | 5 | 5 |
| LRRFIP2 | 13 | 11.92661 | 16271.97 | 3.374414 | 9 | 9 |
| MAGEA10 | 13 | 11.92661 | 7393.198 | 3.156872 | 5 | 5 |
| STAT1 | 13 | 11.92661 | 21774.47 | 5.037478 | 9 | 9 |
| DDX43 | 12 | 11.00917 | 10360.57 | 4.337159 | 2 | 2 |
| GAGE1 | 12 | 11.00917 | 12540.95 | 5.515388 | 2 | 2 |
| GAGE2C | 12 | 11.00917 | 11572.43 | 5.342098 | 3 | 3 |
| MAGEA4 | 12 | 11.00917 | 9790.552 | 4.88818 | 3 | 3 |
| MAP2K5 | 11 | 10.09174 | 10511.43 | 3.676394 | 2 | 2 |
| FADD | 10 | 9.174312 | 18237.53 | 5.423004 | 6 | 6 |
| IGF2BP3 | 9 | 8.256881 | 14223.89 | 6.351528 | 2 | 2 |
| CT47A1 | 8 | 7.33945 | 15453.54 | 6.885753 | 6 | 6 |
| DDX53 | 7 | 6.422018 | 30847.36 | 11.16542 | 4 | 4 |
| | | Penetrance | | | Penetrance | |
| | Mean | Fold | | Frequency | Fold | |
| | Penetrance | Change | Frequency | % | Change | Mean |
| Protein | (Control) | (Control) | Differential | Differential | Differential | (Control) |
| XAGE1D | 10338.27 | 4.058168 | 22 | 19.85321 | 5.619843 | 2547.522 |
| PTPN20A | 12011.96 | 3.729957 | 13 | 11.59633 | 0.153117 | 3220.401 |
| TPM1 | 25399.96 | 4.267092 | 9 | 7.761468 | −1.42116 | 5952.523 |
| CTAG1A | 10141.22 | 4.535687 | 10 | 8.844037 | 5.903878 | 2235.872 |
| RAD23B | 9467.406 | 3.733816 | 9 | 7.844037 | −0.50237 | 2535.585 |
| ZNRD1 | 12658.28 | 2.991589 | 10 | 8.844037 | −0.00263 | 4231.29 |
| CTAG2 | 10221.57 | 4.181798 | 8 | 6.926606 | 4.302857 | 2444.301 |
| LRRFIP2 | 11729.15 | 2.432342 | 4 | 2.926606 | 0.942072 | 4822.161 |
| MAGEA10 | 7423.496 | 3.169809 | 8 | 6.926606 | −0.01294 | 2341.938 |
| STAT1 | 11512.56 | 2.663407 | 4 | 2.926606 | 2.374071 | 4322.495 |
| DDX43 | 8368.541 | 3.503251 | 10 | 9.009174 | 0.833908 | 2388.793 |
| GAGE1 | 7578.361 | 3.33289 | 10 | 9.009174 | 2.182498 | 2273.81 |
| GAGE2C | 7604.188 | 3.510266 | 9 | 8.009174 | 1.831832 | 2166.271 |
| MAGEA4 | 7559.253 | 3.774148 | 9 | 8.009174 | 1.114032 | 2002.903 |
| MAP2K5 | 6913.192 | 2.417902 | 9 | 8.091743 | 1.258492 | 2859.17 |
| FADD | 11120.35 | 3.306683 | 4 | 3.174312 | 2.116321 | 3362.994 |
| IGF2BP3 | 6903.872 | 3.082851 | 7 | 6.256881 | 3.268677 | 2239.444 |
| CT47A1 | 9374.437 | 4.177039 | 2 | 1.33945 | 2.708714 | 2244.278 |
| DDX53 | 17135.53 | 6.202328 | 3 | 2.422018 | 4.963095 | 2762.758 |

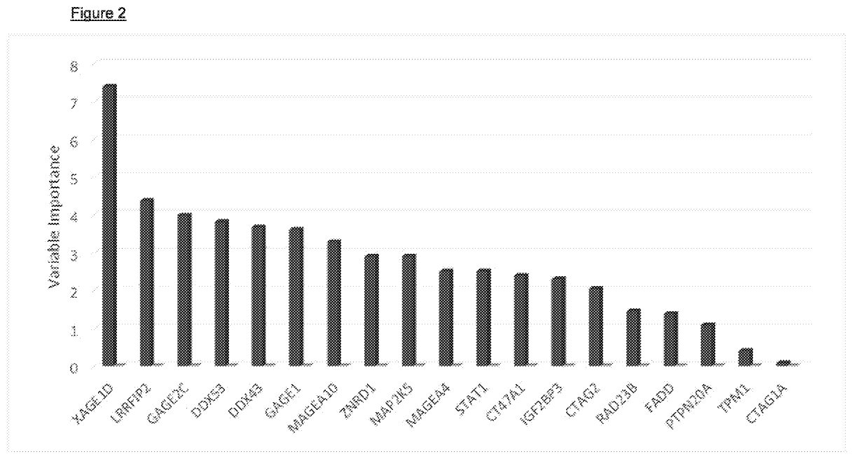

To evaluate the sensitivity of individual biomarkers towards lung cancer, ROG and area under the curve (AUC), 95% confidence intervals and also likelihood ratios were calculated, as set out in Table 2. Variable ranking was performed by using all combination of 19 biomarkers as separate panels and each panel was subjected to recursive feature elimination by generating random forests. The biomarkers were ranked based on random forest estimated variable importance measure derived from each panel (see FIG. 2). Mean variable importance scores determine three core set of biomarkers which are common across all biomarker panels. i.e. XAGE1D, LRRFIP2 and GAGE2C.

TABLE 2

| | | Confidence | Likelihood Ratio | | | Rank |
|---|---|---|---|---|---|---|
| | | Interval | LR— | LR— | Variable | based on |
| Protein | AUC | (95% CI) | Positive | Negative | Importance | Importance |
| XAGE1D | 0.696 | [0.622-0.77] | 7.75 | 0.593 | 7.39 | 1 |
| LRRFIP2 | 0.56 | [0.48-0.641] | 3.068 | 0.735 | 4.37 | 2 |
| GAGE2C | 0.612 | [0.534-0.691] | 13.321 | 0.713 | 3.98 | 3 |
| DDX53 | 0.635 | [0.557-0.714] | 3.229 | 0.614 | 3.81 | 4 |
| DDX43 | 0.641 | [0.564-0.719] | 2.601 | 0.588 | 3.67 | 5 |
| GAGE1 | 0.638 | [0.562-0.715] | 4.239 | 0.676 | 3.61 | 6 |
| MAGEA10 | 0.651 | [0.574-0.728] | 3.817 | 0.598 | 3.28 | 7 |
| ZNRD1 | 0.573 | [0.493-0.653] | 1.462 | 0.665 | 2.9 | 8 |
| MAP2K5 | 0.65 | [0.573-0.726] | 4.037 | 0.654 | 2.9 | 9 |
| MAGEA4 | 0.654 | [0.577-0.73] | 3.176 | 0.553 | 2.5 | 10 |
| STAT1 | 0.554 | [0.472-0.635] | 2.745 | 0.776 | 2.5 | 11 |
| CT47A1 | 0.623 | [0.544-0.702] | 2.382 | 0.594 | 2.39 | 12 |
| IGF2BP3 | 0.621 | [0.542-0.699] | 2.295 | 0.644 | 2.3 | 13 |
| CTAG2 | 0.619 | [0.54-0.698] | 2.637 | 0.664 | 2.04 | 14 |
| RAD23B | 0.59 | [0.51-0.67] | 2.758 | 0.722 | 1.45 | 15 |
| FADD | 0.602 | [0.522-0.681] | 3.633 | 0.737 | 1.38 | 16 |
| PTPN20A | 0.605 | [0.525-0.684] | 2.327 | 0.682 | 1.08 | 17 |
| TPM1 | 0.607 | [0.527-0.686] | 2.22 | 0.641 | 0.4 | 18 |
| CTAG1A | 0.625 | [0.547-0.703] | 2.653 | 0.687 | 0.08 | 19 |

A total of 19 potential autoantibody biomarkers have been identified for diagnosis of NSCLC; namely XAGE1D, PTPN20A, TPM1, CTAG1A, RAD23B, ZNRD1, LRRFIP2, STAT1, MAGEA10, CTAG2, GAGE1, GAGE2C, DDX43, MAGEA4, MAP2K5, FADD, IGF2BP3, CT47A1, DDX53. The Uniprot IDs, description, nucleotide sequence and protein sequence are set out in Table 5 below.

The Immunome array contains >1630 antigens, presented on the array surface in a folded, functional form, as described above. Notably, it would not have been obvious a priori which specific 19 antigens out of the collection of >1630 antigens that were tested would give rise to a measurable autoantibody response that is diagnostic for NSCLC.

In this panel of 19 antigens that correspond to the 19 autoantibody biomarkers, CTAG2 is observed in 25-50% of tumor samples of melanomas, non-small-cell lung carcinomas, bladder, prostate and head and neck cancers. CTAG1A is a tumor cell antigen found in various types of cancers, which makes it a good candidate for a cancer vaccine.

ZNRD1 contains two potential zinc-binding motifs and may play a role in regulation of cell proliferation. The encoded protein may be involved in cancer and human immunodeficiency virus progression.

XAGE1D and MAGEA4 RNA markers have been considered for use in screening of lung neoplasia for detecting presence of lung cancer. In normal tissues, XAGE1D is highly expressed in testis, highly expressed in breast cancer, prostate cancer and many types of lung cancers, including squamous cell carcinoma, small cell carcinoma, non-small cell carcinoma, and adenocarcinoma, as well as in Ewings cell lines, in some Ewings sarcoma patient samples, and in one of one alveolar rhabdomyosarcoma patient sample. MAGEA4 is expressed in many tumors of several types, such as melanoma, head and neck squamous cell carcinoma, lung carcinoma and breast carcinoma, but not in normal tissues except for testes and placenta.

LRRFIP2 is involved in the Wnt signalling pathway and aberrant Wnt signalling underlies a wide range of pathologies in humans. It has been suggested that the Wnt signalling pathway has important functions in stem cell biology, cardiac development and differentiation, angiogenesis, cardiac hypertrophy, cardiac failure and ageing (Rao & Kuhl, 2010). GAGE2C belongs to a family of genes that are expressed in a variety of tumors but not in normal tissues, except for the testis.

PTPN20A is present in many cell lines (at protein level) and is widely expressed. TPM1 is detected in primary breast cancer tissues but undetectable in normal breast tissues in Sudanese patients. Isoform 1 is expressed in adult and fetal skeletal muscle and cardiac tissues, with higher expression levels in the cardiac tissues. Isoform 10 is expressed in adult and fetal cardiac tissues, but not in skeletal muscle.

DDX53, STAT1 and FADD expression levels were elevated in late stage group. DDX53 is a cancer-testis antigen that shows wide expression in many tumours. DDX53 has been reported to interact with EGFR and bind to the promoter sequences of EGFR. Signal transducer and activator of transcription (STAT) 1 is part of the (JAK)/STAT signalling cascade and is best known for its essential role in mediating responses to all types of interferons (IFN). A correlation of STAT1 protein expression levels with poor prognosis, increased invasive and metastatic potential has been reported in three breast cancer studies (Meissl et al., 2017). It was concluded that STAT1 can promote tumour progression, and therefore, it can be a potential marker or indicator of cancer progression (Meissl et al., 2017). Phosphorylation of FADD promotes KRAS induced lung cancer (Bowman et al., 2015). Fas-associated death domain protein (FADD) is the key adaptor molecule transmitting the apoptotic signal delivered by death receptors. It was also reported that the release of FADD by human NSCLC correlates positively with both tumour progression and aggressiveness and could be a new marker of poor prognosis (Cimino et al., 2012).

Cancer-testis antigens are a family of >1000 highly developmentally restricted fetal proteins (Wang et al, 2016; Silva et al., 2017) that are silenced in all somatic tissues except the testes and occasionally placenta, but which can be aberrantly expressed in cancerous tissues, thereby driving an autoantibody response. The Immunome array contains 202 cancer-testis antigens, presented on the array surface in a folded, functional form as described above. Notably, the 19 autoantibody biomarkers of non-small cell lung cancer identified here are significantly enriched for cancer-testis antigens, yet it would not have been obvious a priori which specific 10 cancer-testis antigens out of the collection of 202 that were tested would give rise to a measurable autoantibody response that is diagnostic for NSCLC.

The best method to verify a lung cancer diagnosis involves a multiple biomarker approach rather than a single biomarker approach.

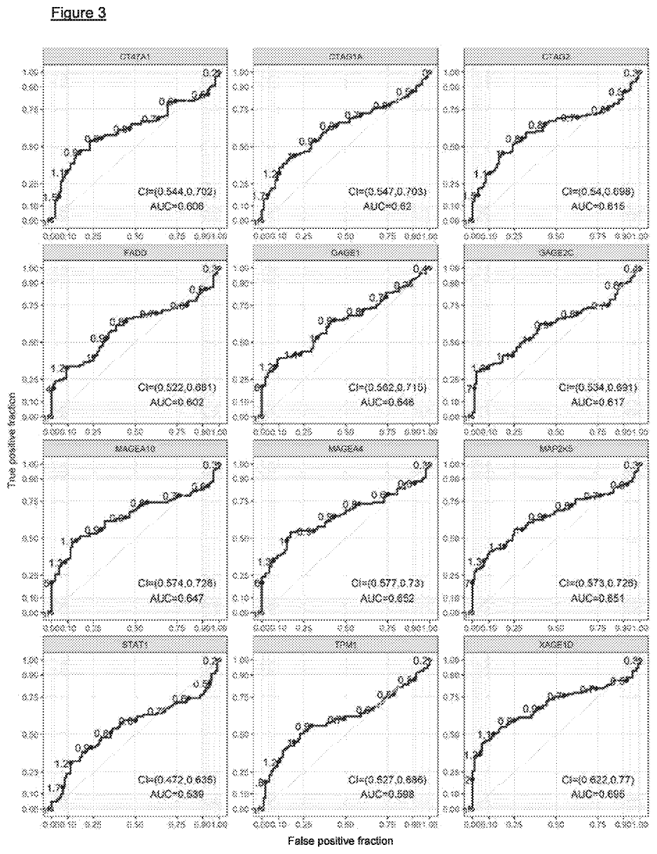

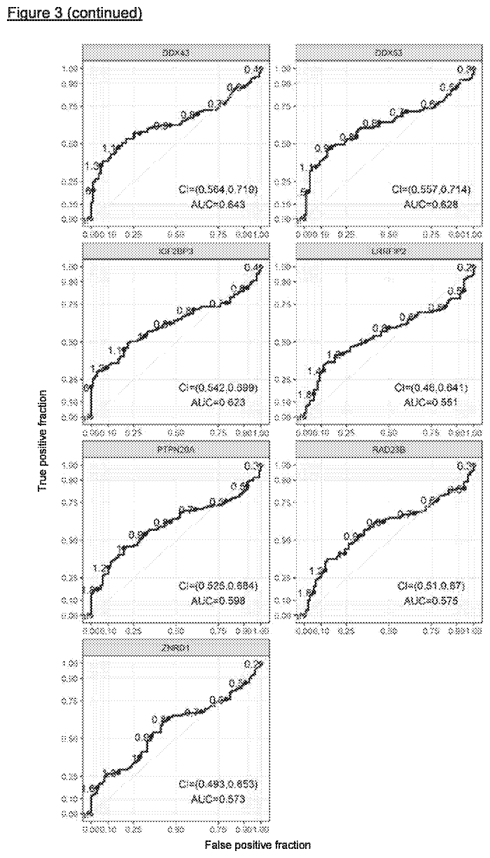

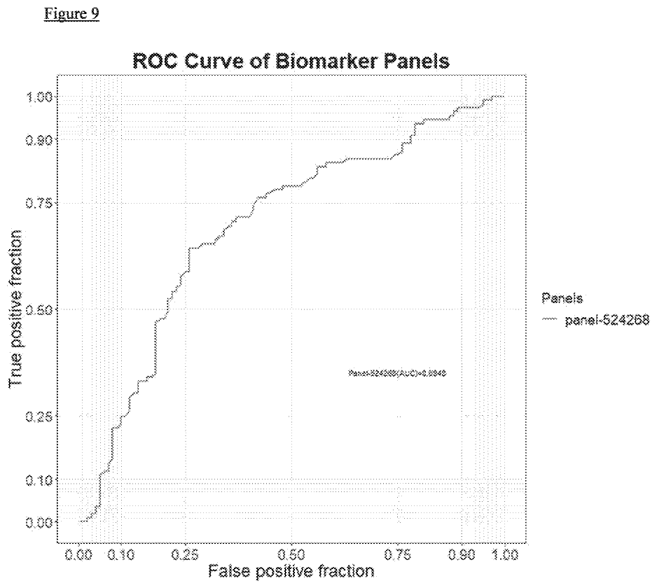

As illustrated in FIG. 3, Receiver operating characteristic (ROC) curves were calculated based on individual fold changes for 19 biomarkers to show sensitivity of biomarkers towards lung cancer patients. Area under curve (AUC), 95% confidence intervals (CI) and Optimal cutoff of individual fold change (Cutoff (IFC) for each were calculated based on the method described in Lopez-Raton et al., (2014) using “OptimalCutpoints” R package. CI and Optimal cut-off values help to determine diagnostic ability of the biomarkers by showing positive or negative test results with lung cancer patients. The ROC curve for the panel of 19 biomarkers is illustrated in FIG. 9.

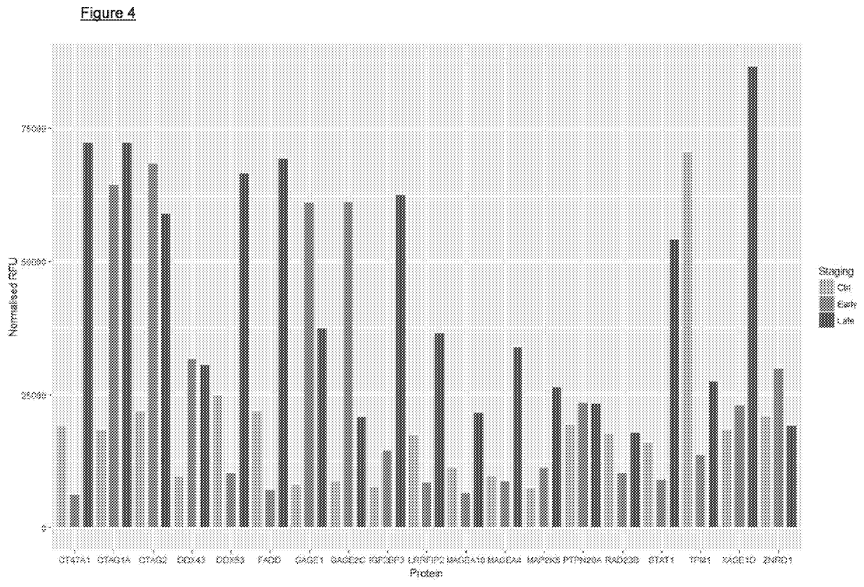

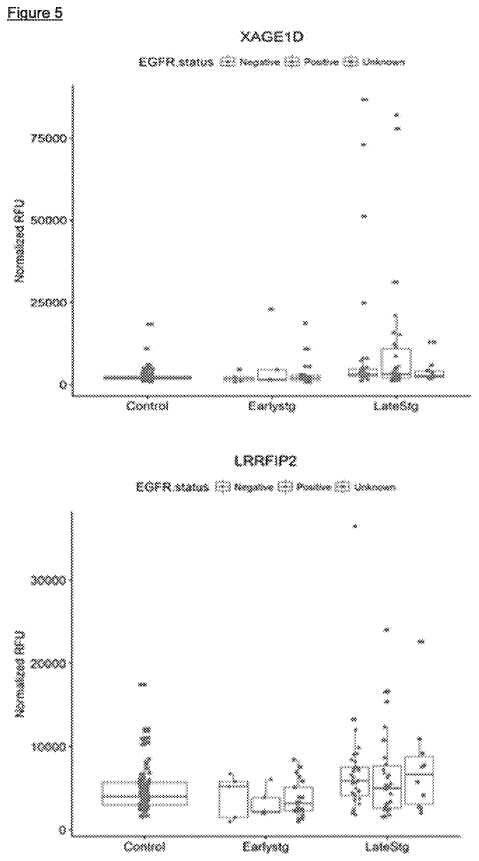

FIG. 4 shows the autoantibody biomarkers determined by Penetrance fold change (pFC) method where age matched controls are considered as baseline to observe the elevated frequencies (2 Foldchange) of individual biomarkers in lung cancer patients. The data was generated from profiling of Normalised RFU values of 19 biomarkers (Table 6) identified by pFC method across healthy controls (Ctrl), Early stage lung cancer patients (Early) and Late stage lung cancer patients (Late).

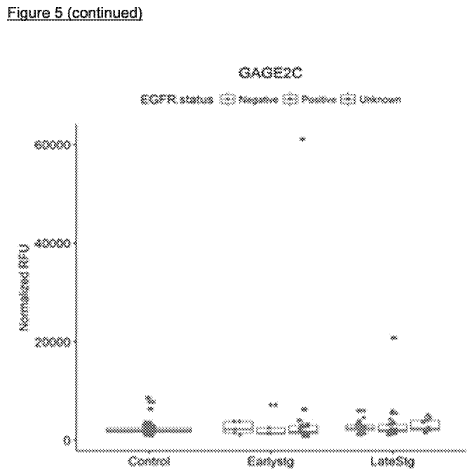

FIG. 5 shows a comparison of the core autoantibody biomarker panel (autoantibodies against antigens XAGE1D, LRRFIP2 and GAGE2C) levels with respect to EGFR status in patients. The data was generated from comparing normalised RFU values of the three core set of biomarkers (identified based on variable ranking using random forest) across healthy controls (Control), Early stage lung cancer patients (Earlystg) and Late stage lung cancer patients (Latestg). Patient cohorts were sub-divided based on EGFR mutation status i.e. Positive (patients with EGFR mutation), negative (patients without EGFR mutation), unknown (patients with unknown EGFR mutation status). An overall elevation of the antigen-specific autoantibody levels was observed in late stage NSCLC compared to early stage NSCLC and control.

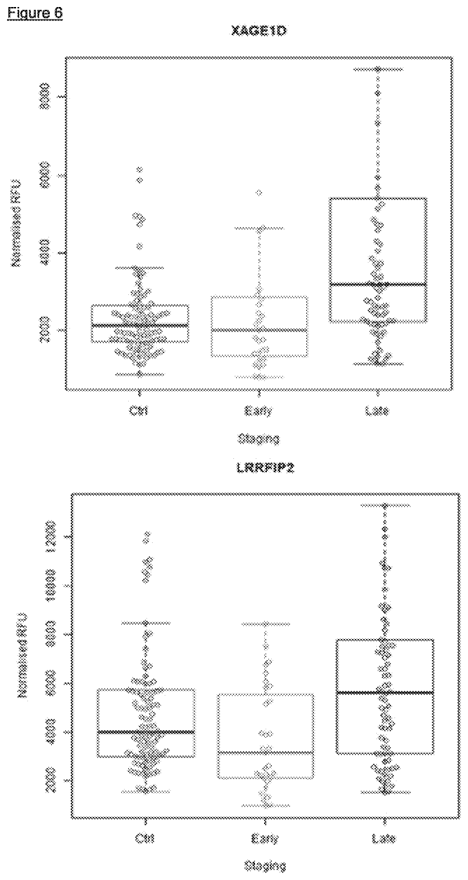

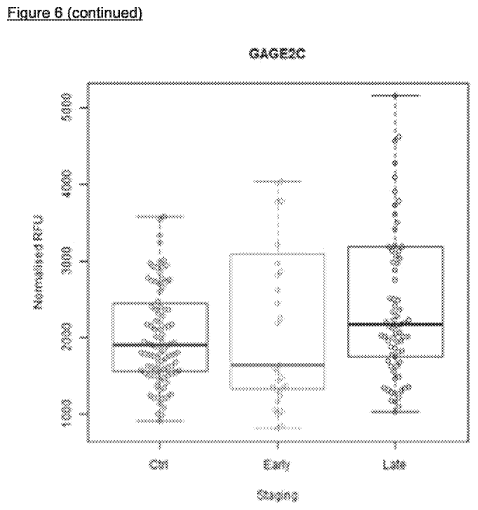

A similar observation is seen in FIG. 6, wherein the data was generated from comparing normalised RFU values of the three core set of biomarkers (identified based on variable ranking using random forest) against healthy controls (Ctrl), Early stage lung cancer patients (Early) and Late stage lung cancer patients (Late).

Furthermore, the elevation of antigen-specific autoantibody levels in late stage NSCLC is independent of EGFR status of patients. In addition, with further reference to FIG. 7, distinct differences in autoantibodies against XAGE1D, CTAG1A, CTAG2, GAGE1 and GAGE2C were observed in Late and Early stage NSCLC compared to the control. Unsupervised clustering of individual fold changes across all healthy controls (Control), Early stage lung cancer patients (EarlyStg) and Late stage lung cancer patients (LateStg) for 19 biomarkers identified by pFC method. Clustering was performed for biomarkers based on Ward's method and distance calculated based on Euclidean distance. The shaded bar on the top of the heatmap represents patient cohorts i.e. Control, Early stage and Late Stage samples.

Validation Study Using a Custom Array Containing 19 Antigens Identified from the Phase 1 Study

### Protein Expression:

Nineteen BCCP-tagged antigens (XAGE1D; CTAG2; CTAG1A; STAT1; DDX53; MAGEA4; IGF2BP3; MAGEA10; LRRFIP2; ZNRD1; PTPN20A; RAD23B; CT47A1; MAP2K5; FADD; GAGE1; DDX43; GAGE2C; & TPM1) identified from the Phase 1 discovery study (see above) were expressed in insect cell cultures as previously described (see above). Cells were harvested and lysed as described above.

### Custom Array Fabrication:

Crude insect cell lysates for each of the 19 BCCP-tagged antigens were aliquoted into separate wells of a source plate and robotically printed on to streptavidin-coated hydrogel slides (Schott HS slides) to form a protein microarray. Each of the 19 antigens were printed in triplicate on one array. Sixteen replica arrays were printed in discrete areas of a 7.5×2.5 cm HS slide. Following printing, arrays were wash as stored as described above.

### Study Cohort:

Plasma samples from an independent cohort of 126 late stage NSCLC patients, 30 early stage NSCLC patients and 83 age-matched healthy controls were used to validate the 19 shortlisted antigens from the Phase 1 study, using the custom array fabricated as described above.

### Sample Preparation, Data Handling and QC:

For each plasma sample, 22.5 μL of the sample was pipetted into 4.5 mL of Serum Assay Buffer (SAB) containing 0.1% v/v Triton X-100, 0.1% w/v BSA in PBS (20° C.) and vortexed to mix three times. Diluted plasma were then assayed on custom protein microarrays, essentially as described above. Briefly, each custom protein microarray was removed from storage buffer using forceps, placed in a slide box containing 200 mL cold SAB and shaken on an orbital shaker at 50 rpm, for 5 minutes. The slides were then placed, array side up, in a slide hybridization chamber with individual plasma which had been diluted as above. All slides were scanned using a barcode scanner and incubated on a horizontal shaker at 50 rpm for 2 hours at 20° C. Each protein microarray slide was then rinsed twice with 30 mL SAB, followed by 200 mL of SAB buffer for 20 minutes on the shaker at 50 rpm at room temperature. All slides were transferred sequentially and in the same orientation. Arrays were then immersed in hybridization solution containing Cy3-rabbit anti-human IgG (diluted 1:1000 in SAB buffer) for 2 hours, with shaking at 50 rpm at 20° C.

After incubation, the slide was washed in 200 mL of SAB buffer, 3 times for 5 minutes with shaking at 50 rpm at room temperature. Excess buffer was removed by immersing the slide in 200 mL of pure water for a few minutes. Slides were then dried for 2 min at 240 g at room temperature and stored at room temperature until scanning. Hybridization signals were measured with a microarray laser scanner (Agilent Scanner) at 10 μm resolution. Fluorescence intensities were detected according to the manufacturer's instructions, whereby each spot is plotted using Agilent Feature Extraction software.

Slide scanning, raw data handling and QC were carried out as described above for the Phase 1 study.

### Data Analysis:

A Penetrance Fold Change analysis was performed for each of the 19 antigens, comparing NSCLC patients and healthy controls, using the method described for the Phase 1 study data analysis. This demonstrated that all 19 antigens had an individual penetrance frequency>10% and a penetrance fold change>2 fold.

The results are summarised below. Table 3 shows the penetrance fold change analysis results for Late Stage NSCLC versus Healthy controls across all 19 antigens from the validation study. Table 4 shows the penetrance fold change analysis results for Early Stage NSCLC versus Healthy controls across all 19 antigens from the validation study.

TABLE 3

| | Penetrance | Penetrance | Mean | Penetrance | |
|---|---|---|---|---|---|
| | Frequency | Frequency % | Penetrance | Fold Change | Mean (Healthy |
| Protein | (Late Stage) | (Late Stage) | (Late Stage) | (Late Stage) | Control) |
| XAGE1D | 33 | 26.19 | 2103.27 | 4.32 | 486.84 |
| CTAG2 | 26 | 20.63 | 1896.52 | 3.93 | 482.11 |
| CTAG1A | 25 | 19.84 | 3649.61 | 8.28 | 440.94 |
| STAT1 | 21 | 16.67 | 1219.31 | 2.70 | 451.38 |
| DDX53 | 20 | 15.87 | 7340.65 | 13.67 | 536.89 |
| MAGEA4 | 20 | 15.87 | 2722.11 | 6.17 | 440.83 |
| IGF2BP3 | 20 | 15.87 | 1428.20 | 3.13 | 455.58 |
| MAGEA10 | 20 | 15.87 | 1274.75 | 2.72 | 468.93 |
| LRRFIP2 | 20 | 15.87 | 1366.38 | 2.65 | 516.39 |
| ZNRD1 | 19 | 15.08 | 1440.51 | 2.96 | 486.98 |
| PTPN20A | 19 | 15.08 | 1244.62 | 2.67 | 466.38 |
| RAD23B | 18 | 14.29 | 1211.94 | 2.88 | 421.08 |
| CT47A1 | 18 | 14.29 | 1330.74 | 2.74 | 486.50 |
| MAP2K5 | 17 | 13.49 | 1227.44 | 2.86 | 429.29 |
| FADD | 16 | 12.70 | 1362.23 | 2.79 | 487.64 |
| GAGE1 | 16 | 12.70 | 1741.70 | 2.75 | 633.18 |
| DDX43 | 16 | 12.70 | 1274.21 | 2.70 | 471.32 |
| GAGE2C | 14 | 11.11 | 1441.22 | 2.93 | 491.95 |
| TPM1 | 13 | 10.32 | 3066.49 | 2.81 | 1090.13 |

TABLE 4

| | Penetrance | Penetrance | | Penetrance | |
|---|---|---|---|---|---|
| | Frequency | Frequency % | Mean Penetrance | Fold Change | Mean |
| Protein | (EarlyStage) | (EarlyStage) | (EarlyStage) | (EarlyStage) | (Control) |
| RAD23B | 8 | 26.67 | 992.75 | 2.36 | 421.08 |
| GAGE1 | 5 | 16.67 | 1498.35 | 2.37 | 633.18 |
| DDX43 | 5 | 16.67 | 1095.95 | 2.33 | 471.32 |
| XAGE1D | 4 | 13.33 | 3219.83 | 6.61 | 486.84 |
| LRRFIP2 | 4 | 13.33 | 1343.66 | 2.60 | 516.39 |
| MAGEA4 | 4 | 13.33 | 1141.63 | 2.59 | 440.83 |
| MAGEA10 | 4 | 13.33 | 1121.54 | 2.39 | 468.93 |
| GAGE2C | 4 | 13.33 | 1160.49 | 2.36 | 491.95 |
| CT47A1 | 4 | 13.33 | 1129.55 | 2.32 | 486.50 |
| PTPN20A | 4 | 13.33 | 1075.17 | 2.31 | 466.38 |
| MAP2K5 | 4 | 13.33 | 984.14 | 2.29 | 429.29 |
| STAT1 | 4 | 13.33 | 1033.02 | 2.29 | 451.38 |
| ZNRD1 | 4 | 13.33 | 1109.92 | 2.28 | 486.98 |
| TPM1 | 3 | 10.00 | 3102.74 | 2.85 | 1090.13 |
| CTAG1A | 3 | 10.00 | 1088.11 | 2.47 | 440.94 |
| IGF2BP3 | 3 | 10.00 | 1123.99 | 2.47 | 455.58 |
| CTAG2 | 3 | 10.00 | 1145.82 | 2.38 | 482.11 |
| DDX53 | 2 | 6.67 | 1260.99 | 2.35 | 536.89 |
| FADD | 2 | 6.67 | 1095.78 | 2.25 | 487.64 |

The performances of biomarker panels were validated by a Random Forest—Recursive feature elimination (RF-RFE) algorithm which is a backwards selection, iterative process used to select the best subset of biomarkers for the classification of NSCLC. Validation of the selected biomarkers involves using a training and testing set for model generation and performance evaluation before using an independent validation set to validate the final performance of the models.

During model generation, all possible combinations of 19 biomarkers were generated and the individual fold change values based on the biomarker combinations were used as inputs for model generation. Data from the phase I (209 samples) were separated into training (⅔rds) and test (⅓rd) datasets. Training using the RF-RFE was done using default parameters with 5-fold cross validation and panel size being fixed to the number of biomarkers in each model. The generated models were used to predict both the testing and validation sets to evaluate the performance of the panels in the stratification of NSCLC. All recursive feature elimination and Random Forest analyses were performed using the caret (Kuhn, 2008 (https://www.jstatsoft.org/article/view/v028i05)) package in R.

The performance of the RF-RFE models on the training dataset are summarized in Table 7 which includes results for the performance of the core biomarkers, 19 biomarkers and the top 20 panels based on descending AUC values.

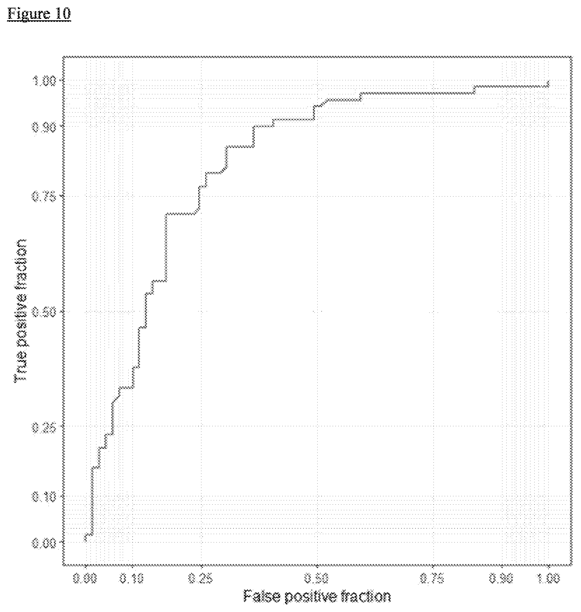

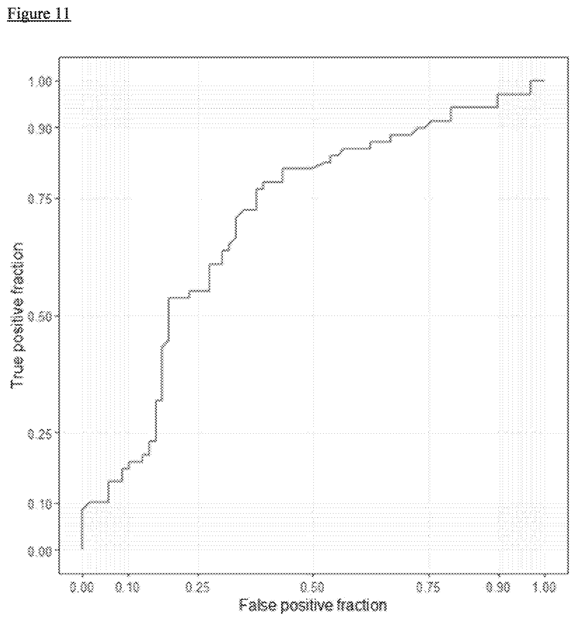

Results have demonstrated that the panel of 7 biomarkers which includes the core biomarkers (XAGE1D, LRRFIP2, GAGE2C) outperform a panel of 19 biomarkers with Sensitivity and Specificity of 0.753 and 0.721 compared to 0.680 and 0.652 respectively. FIGS. 10 and 11 show the ROC curves for the best panel of 7 biomarkers (XAGE1D, LRRFIP2, MAGEA10, GAGE2C, STAT1, ZNRD1, RAD23B; with an AUC of 0.818), and the 19 biomarkers (with an AUC of 0.702) respectively.

Extracellular vesicles can be divided into three main categories, namely apoptotic bodies, microvesicles, and exosomes. Exosomes are the smallest extracellular vesicles which are naturally secreted by almost every cell type and can be found in almost all biological fluids including blood, serum, plasma, urine, saliva, amniotic fluid, cerebrospinal fluid, breast milk, semen and bile. In general, cells release exosomes via two mechanisms. The classic pathway involves the formation of intraluminal vesicles within multivesicular endosomes. In turn, the membrane of multivesicular endosomes fuses with the plasma membrane, resulting in the release of intraluminal vesicles. When secreted, intraluminal vesicles are called exosomes. Alternatively, the direct pathway involves the release of vesicles, indistinguishable from exosomes, directly from the plasma membrane (van der Pol et al. 2012). Interestingly, exosomes from cancer cells have been shown to promote angiogenesis, modulate the immune system and remodel the surrounding parenchymal tissue, all factors supporting tumor progression (Hessvik and Llorente, 2018).

Exosome samples from patients with NSCLC and healthy controls were collected and isolated using Invitrogen Total Exosome Isolation (from plasma) kit (Thermo Fisher Scientific.) based on the established protocol from the manufacturer.

The discovery of these autoantibody biomarkers using exosome samples can be more disease-specific and meaningful as they contain membrane-bound proteins that reflect their originating cell. In cancer, exosomes have been shown to be implicated in the crosstalk between tumour cells and normal cells thereby facilitating the malignant process. Several studies have found exosomes to be promising as diagnostic and prognostic markers (Sanfeld-Paulsen et al., 2016).

It will be appreciated by persons skilled in the art that the present invention may also include further additional modifications made to the system which does not affect the overall functioning of the system.

### REFERENCES

Bolstad, B. M. et al (2013) A comparison of normalization methods for high density oligonucleotide array data based on variance and bias. Bioinformatics 19(2), 185-93.
Cimino, Y. et al. (2012). FADD protein release mirrors the development and aggressiveness of human non-small cell lung cancer. British Journal of Cancer, 106, 1989-1996.
Hessvik N P and Llorente A (2018). Current knowledge on exosome biogenesis and release. Cell Mol Life Sci., 75(2): 193-208.
López-Ratón, M., Rodriguez-Álvarez, M. X., Cadarso-Suárez, C., & Gude-Sampedro, F. (2014). OptimalCutpoints: an R package for selecting optimal cutpoints in diagnostic tests. Journal of Statistical Software, 61(8), 1-36
Maniatis T. et al (1989) Molecular Cloning: A Laboratory Manual. Cold Spring Harbor Press
Meissl et al. (2017). The good and the bad faces of STAT1 in solid tumours. Cytokine, vol 89, pages 12-20.
Sanfeld-Paulsen, B. et al. (2016). Exosomal proteins as prognostic biomarkers in non-small cell lung cancer. Molecular Oncology, Vol 10, issue 10, pages 1595-1602.
Silva et al. (2017). Genome-wide identification of cancer/testis genes and their association with prognosis in a pan-cancer analysis. Oncotarget 8, 92966.
van der Pol, Edwin et alt (2012). Pharmacological reviews Classification, Functions, and Clinical Relevance of Extracellular Vesicles.
Rao, T P & Kuhl, M. (2010). An updated overview on Wnt signalling pathways: a prelude for more. Circ Res. 25; 106(12): 1798-806.
Wang et al. (2016). Systematic identification of genes with a cancer-testis expression pattern in 19 cancer types. Nature Communications 7, 10499.
www.genecards.org

TABLE 5

| Protein Name | UniprotID | Description |
|---|---|---|
| XAGE1D | Q9HD64 | >P003055_Q211_Q211_tube_XAGE1_9503_0_NM_020411.2_0_Q9HD64_0_Insert |
| | | sequence is gene optimized by GeneArt_0_0_0 |

Nucleotide Sequence (Seq ID No. 1):
ATGGAATCCCCCAAGAAGAAGAACCAGCAGCTGAAGGTCGGAATCCTGCACCTGGGTTCCCGTCAGAAGAAGA
TCCGTATCCAGCTGCGTTCCCAGTGCGCTACCTGGAAGGTCATCTGCAAGTCCTGCATCTCCCAGACCCCCGG
TATCAACCTGGACCTGGGCTCCGGTGTCAAGGTCAAGATCATCCCCAAGGAAGAACACTGGAAGATGCCCGAG
GCTGGCGAGGAACAGCCCCAGGTG
Protein Sequence (Seq ID No. 20):
MESPKKKNQQLKVGILHLGSRQKKIRIQLRSQCATWKVICKSCISQTPGINLDLGSGVKVKIIPKEEHCKMPEAGEEQPQV

| | | |
|---|---|---|
| LRRFIP2 | Q9Y608 | >P001894_Q305_Q305p2_LRRFIP2_9209_Homo sapiens leucine rich repeat |
| | | (in FLII) interacting protein |
| | | 2_BC053668.1_AAH53668.1_Q9Y608_0_0_1203_0_1200 |

Nucleotide Sequence (Seq ID No. 2):
ATGGGGACTCCTGCTTCTGGAAGGAAAAGAACACCTGTGAAAGACCGATTTTCTGCAGAAGATGAAGCTTTGAG
TAACATTGCCAGAGAGGCAGAGGCAAGGCTGGCAGCAAAACGGGCTGCCCGGGCAGAAGCAAGAGATATACG
CATGAGAGAACTGGAACGACAACAAAAAGAGTTGGATGAAAAATCTGACAAACAGTATGCTGAAAATTATACAA
GACCTTCATCTCGAAATTCTGCCTCAGCAACAACCCCTCTAAGTGGAAACTCATCCAGACGAGGAAGTGGGGAC
ACCAGCAGCTTAATAGATCCAGACACTTCATTAAGTGAATTGCGGGAATCTTTGTCTGAAGTGGAAGAAAAATAC
AAGAAAGCCATGGTTTCCAATGCACAGTTAGACAATGAGAAGAACAATTTGATCTACCAAGTAGACACACTCAAG
GATGTTATTGAAGAGCAGGAGGAACAGATGGCAGAATTTTATAGAGAAAATGAAGAAAAATCAAAGGAGTTAGA
AAGGCAGAAACATATGTGTAGTGTGCTGGAGCATAAGATGGAAGAACTTAAAGAAGGCCTGCGGCAAAGAGAT
GAGCTTATTGAGAAACATGGCTTAGTTATAATCCCCGATGGCACTCCCAATGGTGATGTCAGTCATGAACCAGT
GGCTGGAGCCATCACTGTTGTGTCTCAGGAAGCTGCTCAGGTCTTGGAGTCAGCAGGAGAAGGGCCATTAGAT
GTAAGGCTACGAAAACTTGCTGGAGAGAAGGAAGAACTACTGTCACAGATTAGAAAACTGAAGCTTCAGTTAGA
GGAGGAACGACAGAAATGCTCCAGGAATGATGGCACAGTGGGTGACCTGGCAGGACTGCAGAATGGCTCAGA
CTTGCAGTTCATCGAAATGCAGAGAGATGCCAATAGACAAATTAGCGAATACAAATTTAAGCTTTCAAAAGCAGA
ACAGGATATAACTACCTTGGAGCAAAGTATTAGGCGGCTTGAGGGACAGGTTCTGAGATATAAAACTGCTGCTG
AGAATGCTGAGAAAGTTGAAGATGAATTGAAAGCAGAAAAACGGAAGCTACAACGAGAGTTACGAACAGCACTG
GACAAGATTGAGGAGATGGAGATGACCAACAGCCACCTGGCCAAGCGGCTGGAGAAGATGAAGGCCAATAGG
ACAGCACTTCTGGCCGAGCAG
Protein Sequence (Seq ID No.21)
MGTPASGRKRTPVKDRFSAEDEALSNIAREAEARLAAKRAARAEARDIRMRELERQQKELDEKSDKQYAENYTAPS
SRNSASATTPLSGNSSRRGSGDTSSLIDPDTSLSELRESLSEVEEKYKKAMVSNAQLDNEKNNLIYQVDTLKDVIEEQ
EEQMAEFYRENEEKSKELERQKHMCSVLQHKMEELKEGLRQRDELIEKHGLVIIPDGTPNGDVSHEPVAGAITVVSQ
EAAQVLESAGEGPLDVRLRKLAGEKEELLSQIRKLKLQLEEERQKCSRNDGTVGDLAGLQNGSDLQFIEMQRDANR
QISEYKFKLSKAEQDITTLEQSISRLEGQVLRYKTAAENAEKVEDELKAEKRKLQRELRTALDKIEEMEMTNSHLAKRL
EKMKANPTALLAQQ

| | | |
|---|---|---|
| GAGE2C | Q13066 | >P003037_Q211_Q211_tube_GAGE2C_2574_0_NM_001472.2_0_Q13066_0_Insert |
| | | sequence is gene optimized by GeneArt_0_0_0 |

Nucleotide Sequence (Seq ID No. 3):
ATGTCCTGGCGTGGTCGTTCCACCTACCGTCCCCGTCCTCGTCGTTACGTCGAGCCCCCCGAGATGATCGGTC
CCATGCGTCCCGAGCAGTTCTCCGACGAGGTCGAGCCCGCTACCCCCGAGGAAGGCGAGCCTGCTACTCAGC
GTCAGGACCCCGCTGCTGCTCAAGAGGGCGAGGACGAGGGCGCTTCCGCTGGCCAGGGTCCTAAGCCCGAG
GCTCACTCCCAAGAGCAGGGTCACCCCCAGACCGGTTGCGAGTGCGAGGACGGTCCCGACGGTCAAGAGATG
GACCCCCCCAACCCTGAGGAAGTCAAGACCCCCGAAGAGGGCGAAAAGCAGTCCCAGTGC
Protein Sequence (Seq ID No. 22):
MSWRGRSTYRPRPRRYVEPPEMIGPMRPEQFSDEVEPATPEEGEPATQRQDPAAAQEGEDEGASAGQGPKPEAH
SQEQGHPQTGCECEDGPDGQEMDPPNPEEVKTPEEGEKQSQC

| | | |
|---|---|---|
| DDX53 | Q86TM3 | >P003022_Q211_Q211_tube_DDX53_168400_0_NM_182699.2_0_Q86TM3_0_Insert |
| | | sequence is gene optimized by GeneArt_0_0_0 |

Nucleotide Sequence(Seq ID No. 4):
ATGTCCCACTGGGCTCCCGAGTGGAAGCGTGCTGAGGCTAACCCCCGTGACCTGGGCGCTTCTTGGGACGTG
CGCGGTTCCCGTGGTAGCGGTTGGAGCGGTCCCTTCGGTCACCAAGGTCCCCGTGCTGCTGGTTCCCGCGAG
CCCCCCCGTGCTTCAAGATCAAGAACAACATGGTCGGAGTGGTCATCGGTTACTCCGGTTCCAAGATCAAGG
ACCTGCAGCACTCCACCAACACCAAGATCCAGATCATCAACGGCGAGTCCGAGGCTAAGGTCCGCATCTTCGG
CAACCGCGAGATGAAGGCTAAGGCCAAGGCTGCTATCGAGACTCTGATCCGCAAGCAAGAGTCCTACAACTCC
GAGTCCTCCGTGGACAACGCTGCTTCCCAGAGCCCCATCGGTCGTAACCTGGGTCGTAACGACATCGTGGGC
GAGGCTGAGCCCCTGTCCAACTGGGACCGTATCCGCGCTGCTGTGGTCGAGTGCGAGAAGCGCAAGTGGGCT
GACCTGCCCCCCGTGAAGAAGAACTTCTACATCGAGTCCAAGGCTACCTCCTGCATGTCCGAGATGCAAGTGA
TCAACTGGCGCAAGGAAAACTTCAACATCACTTGCGACGACCTGAAGTCCGGCGAGAAGCGTCTGATCCCCAA
GCCTACCTGCCGTTTCAAGGACGCTTTCCAGCAGTACCCCGACCTGCTGAAGTCCATCATCCGTGTGGGTATC
GTGAAGCCCACCCCCATCCAGTCCCAGGCTTGGCCAATCATCCTGCAGGGTATCGACCTGATCGTGGTGGCTC
AGACCGGCACCGGCAAGACCCTGTCCTACCTGATGCCCGGTTTCATCCACCTGGACTCCCAGCCCATCTCCCG
CGAGCAGCGTAACGGTCCCGGCATGCTGGTGCTGACCCCTACCCGTGAACTGGCTCTGCACGTCGAGGCTGA
GTGCTCCAAGTACTCCTACAAGGGCCTGAAGTCTATCTGCATCTACGGTGGTCGTAACCGTAACGGCCAGATC
GAGGACATCTCCAAGGGTGTCGACATCATCATTGCTACCCCCGGTCGTCTGAACGACCTGCAGATGAACAACT
CCGTGAACCTGCGTTCCATCACCTACCTGGTCATCGACGAGGCTGACAAGATGCTGGACATGGAGTTCGAGCC
CCAGATCCGCAAGATCCTGCTGGACGTGCGTCCCGACCGTCAGACCGTGATGACCTCCGCTACCTGGCCCGA
CACCGTGCGTCAGCTGGCTCTGTCTTACCTGAAGGACCCCATGATCGTGTACGTGGGCAACCTGAACCTGGTG
GCTGTGAACACCGTGAAGCAGAACATCATCGTGACCACCGAGAAGGAAAAGAGGGCTCTGACCCAAGAGTTCG
TCGAGAACATGTCCCCCAACGACAAGGTCATCATGTTCGTGTCCCAGAAGCACATTGCTGACGACCTGTCCTCC
GATTTCAACATCCAAGGCATCTCCGCTGAGTCCCTGCACGGCAACTCCGAGCAGTCCGACCAAGAGCGTGCTG
TCGAGGACTTCAAGTCCGGCAACATCAAGATCCTCATCACCACCGACATCGTGTCCCGTGGCCTGGACCTGAA
CGACGTGACCCACGTGTACAACTACGACTTCCCCCGTAACATCGACGTGTACGTGCACCGTGTGGGTTACATC
GGTCGCACCGGAAAGACCGGAACCTCCGTGACCCTGATCACCCAGCGCGACTCCAAGATGGCTGGCGAGCTG
ATCAAGATCTTGGACCGTGCTAACCAGTCCGTGCCCGAGGACCTGGTGGTCATGGCTGAGCAGTACAAGCTGA
ACCAGCAGAAGCGTCACCGCGAGACTCGTTCCCGCAAGCCCGGACAGCGTCGCAAGGAGTTCTACTTCCTGTC
C
Protein Sequence(Seq ID No. 23):
MSHWAPEWKRAEANPRDLGASWDVRGSRGSGWSGPFGHQGPRAAGSREPPLCFKIKNNMVGVVIGYSGSKIKDL
QHSTNTKIQIINGESEAKVRIFGNREMKAKAKAAIETLIRKQESYNSESSVDNAASQTPIGRNLGRNDIVGEAEPLSNW
DRIRAAVVECEKRKWADLPPVKKNFYIESKATSCMSEMQVINWRKENFNITCDDLKSGEKRLIPKPTCRFKDAFQQY
PDLLKSIIRVGIVKPTPIQSQAWPIILQGIDLIVVAQTGTGKTLSYLMPGFIHLDSQPISREQRNGPGMLVLTPTRELALH
VEAECSKYSYKGLKSICIYGGRNRNGQIEDISKGVDIIIATPGRLNDLQMNNSVNLRSITYLVIDEADKMLDMEFEPQIR
KILLDVRPDRQTVMTSATWPDTVRQLALSYLKDPMIVYVGNLNLVAVNTVKQNIIVTTEKEKRALTQEFVENMSPNDK
VIMFVSQKHIADDLSSDFNIQGISAESLHGNSEQSDQERAVEDFKSGNIKILITTDIVSRGLDLNDVTHVYNYDFPRNID
VYVHRVGYIGRTGKTGTSVTLITQRDSKMAGELIKILDRANQSVPEDLVVMAEQYKLNQQKRHRETRSRKPGQRRKE
FYFLS

| | | |
|---|---|---|
| DDX43 | Q9NXZ2 | >P003043_Q211_Q211_tube_DDX43_55510_0_NM_018665.2_0_Q9NXZ2_0_Insert |
| | | sequence is gene optimized by GeneArt_0_0_0 |

Nucleotide Sequence(Seq ID No. 5):
ATGTCCCACCACGGTGGTGCTCCCAAGGCTTCCACCTGGGTGGTGGCTTCCCGTCGTTCCTCCAGCGTGTCCC
GTGCTCCCGAGCGTCGTCCCGCTGAGGAACTGAACCGTACCGGTCCCGAGGGCTACTCCGTGGGTCGTGGTG
GTCGTTGGCGTGGCACCTCTCGTCCCCCTGAGGCTGTGGCTGCTGGTCACGAGGAACTGCCCCTGTGCTTCG
CTCTGAAGTCCCACTTCGTGGGTGCTGTGATCGGTCGCGGTGGTTCCAAGATCAAGAACATCCAGTCCACCAC
CAACACCACCATCCAGATCATCCAAGAGCAGCCCGAGTCCCTGGTCAAGATCTTCGGTTCCAAGGCTATGCAG
ACCAAGGCTAAGGCTGTGATCGACAACTTCGTGAAGAAGCTGGAAGAGAACTACAACTCCGAGTGCGGTATCG
ACACCGCTTTCCAGCCCTCCGTGGGCAAGGACGGTTCCACCGACAACAACGTGGTGGCTGGCGACCGTCCCC
TGATCGACTGGGACCAGATCCGTGAAGAGGGCCTGAAGTGGCAAAAGACCAAGTGGGCTGACCTGCCCCCCA
TCAAGAAGAACTTCTACAAGGAATCCACCGCTACCTCCGCTATGTCCAAGGTCGAGGCTGACTCCTGGCGCAA
GGAAAACTTCAACATCACCTGGGACGACCTGAAGGACGGCGAGAAGCGTCCCATCCCCAACCCTACCTGCACC
TTCGACGACGCTTTCCAGTGCTACCCCGAAGTGATGGAAAACATCAAGAAGGCTGGTTTCCAGAAGCCCACCC
CCATCCAGTCCCAGGCTTGGCCCATCGTGCTGCAGGGTATCGACCTGATCGGTGTCGCTCAGACCGGCACCG
GCAAGACCCTGTGCTACCTGATGCCCGGTTTCATCCACCTGGTGCTGCAGCCCTCCCTGAAGGGGCAGCGTAA
CCGTCCCGGCATGCTGGTGCTGACCCCTACCCGCGAACTGGCTCTGCAGGTCGAGGGCGAGTGCTGCAAGTA
CTCCTACAAGGGCCTGCGTTCCGTGTGCGTGTACGGTGGTGGCAACCGTGACGAGCAGATCGAGGAACTCAA
GAAGGGTGTCGACATCATCATCGCTACCCCCGGTCGTCTGAACGACCTGCAGATGTCCAACTTCGTCAACCTG
AAGAACATCACCTACCTGGTCCTGGACGAGGCTGACAAGATGCTGGACATGGGTTTCGAGCCCCAGATCATGA
AGATCCTGCTGGACGTGCGTCCCGACCGTCAGACCGTGATGACCTCCGCTACCTGGCCCCACTCCGTGCACC
GTCTGGCTGAGTCCTACCTGAAGGAACCCATGATCGTGTACGTGGGCACCCTGGACCTGGTGGCTGTGTCCTC
CGTGAAGCAGAACATCATCGTGACCACCGAGGAAGAGAAGTGGTCCCACATGCAGACTTTCTTGCAGTCCATG
TCCTCTACCGACAAGGTCATCGTGTTCGTGTCCCGCAAGGCTGTCGCTGACCACCTGTCCTCCGACCTGATCC
TGGGCAACATCTCCGTCGAGTCCCTGCACGGCGACCGCGAGCAGCGTGACCGCGAGAAGGCTCTCGAGAACT
TCAAGACCGGCAAGGTCCGCATCCTGATCGCTACCGACCTGGCTTCCCGCGGACTGGACGTGCACGACGTGA
CCCACGTGTACAACTTCGACTTCCCCCGTAACATCGAGGAATACGTGCAGCGTATCGGTCGTACCGGTCGTGC
TGGTCGCACCGGTGTCTCCATCACCACCCTGACCCGTAACGACTGGCGTGTGGCTTCCGAGCTGATCAACATC
CTCGAGCGTGCTAACCAGTCCATCCCCGAGGAACTGGTGTCTATGGCTGAGCGTTTCAAGGCTCACCAGCAAA
AGCGCGAGATGGAACGCAAGATGGAACGTCCCCAGGGTCGTCCCAAGAAGTTCCAC
Protein Sequence(Seq ID No. 24):
MSHHGGAPKASTWVVASRRSSTVSRAPERRPAEELNRTGPEGYSVGRGGRWRGTSRPPEAVAAGHEELPLCFAL
KSHFVGAVIGRGGSKIKNIQSTTNTTQIIQEQPESLVKIFGSKAMQTKAKAVIDNFVKKLEENYNSECGIDTAFQPSVG
KDGSTDNNVVAGDRPLIDWDQIREEGLKWQKTKWADLPPIKKNFYKESTATSAMSKVEADSWRKENFNITWDDLKD
GEKRPIPNPTCTFDDAFQCYPEVMENIKKAGFQKPTPIQSQAWPIVLQGIDLIGVAQTGTGKTLCYLMPGFIHLVLQPS
LKGQRNRPGMLVLTPTRELALGVEGECCKYSYKGLRSVCVYGGGNRDEQIEELKKGVDIIIATPGRLNDLQMSNFVN
LKNITYLVLDEADKMLDMGFEPQIMKILLDVRPDRQTVMTSATWPHSVHRLAQSYLKEPMIVYVGTLDLVAVSSVKQN
IIVTTEEEKWSHMQTFLGSMSSTDKVIVFVSRKAVADHLSSDLILGNISVESLHGDREQRDREKALENFKTGKVRILIAT
DLASRGLDVHDVTHVYNFDFPRNIEEYVHRIGRTGRAGRTGVSITTLTRNDWRVASELINILERANQSIPEELVSMAE
RFKAHQQKREMERKMERPQGRPKKFH

| | | |
|---|---|---|
| GAG1 | Q13065 | >P003036_Q211_Q211_tube_GAG1_2543_0_NM_001040663.2_0_Q13068_0_Insert |
| | | sequence is gene optimized by GeneArt_0_0_0 |

Nucleotide Sequence(Seq ID No. 6):
ATGTCCTGGCGTGGTCGTTCCACCTACTACTGGCCCCGTCCCCGTCGTTACGTGCAGCCCGCCGAGATGATCG
GTCCCATGCGTCCCGAGCAGTTCTCCGACGAGGTCGAGCCCGCTACCCCCGAGGAAGGCGAGCCTGCTACTC
AGCGTCAGGACCCCGCTGCTGCTCAAGAGGGCGAGGACGAGGGCGCTTCCGCTGGCCAGGGTCCTAAGCCC
GAGGCTGACTCCCAAGAGCAGGGTCACCCCCAGACCGGTTGCGAGTGCGAGGACGGTCCCGACGGTCAAGA
GATGGACCCCCCCAACCCTGAGGAAGTCAAGACCCCCGAAGAGGGCGAAGGCCAGTCCCAGTGC
Protein Sequence(Seq ID No. 25):
MSWRGRSTYYWPRPRRYVQPPEMIGPMRPEQFSDEVEPATPEEGEPATQRQDPAAAQEGEDEGASAGQGPKPE
ADSQEQGHPQTGCECEDGPDGQEMDPPNPEEVKTPEEEMRSHYVAQTGILWLLMNNCFLNLSPRKP

| | | |
|---|---|---|
| MAGEA10 | P43363 | >P003053_Q211_Q211_tube_MAGEA10_4109_0_NM_001011543.1_0_P43363_0_Insert |
| | | sequence is gene optimized by GeneArt_0_0_0 |

Nucleotide Sequence(Seq ID No. 7):
ATGCCCCGTGCTCCCAAGCGTCAGCGTTGCATGCCCGAAGAGGACCTGCAGTCCCAGTCCGAGACTCAGGGC
CTCGAGGGTGCTCAGGCTCCCCTGGCTGTGGAAGAGGACGCTTCCAGCTCTACCTCTACCTCCTGCAGCTTCC
CCAGCAGCTTCCCATCCTCCAGCTCCTCTAGCTCCTCCTCCTGCTACCCCCTGATCCCCTCCACCCCCGAGGA
AGTGTCCGCTGACGACGAGACTCCCAACCCCCCCCAGTCCGCTCAGATCGCTTGCTCCTCCCCCTCCGTGGTG
GCTTCCCTGCCTCTGGACCAGTCCGACGAGGGTTCCAGCTCCCAGAAGGAAGAGTCCCCCAGCACCCTGCAG
GTCCTGCCCGACTCCGAGTCCCTGCCCCGTTCCGAGATCGACGAGAAGGTTACAGACCTGGTGCAGTTCCTGC
TGTTCAAGTACCAGATGAAGGAACCCATCACCAAGGCTGAGATCCTCGAGTCCGTGATCAAGAACTACGAGGA
CCACTTCCCCCTGCTGTTCTCCGAGGCTTCCGAGTGCATGCTGCTGGTGTTCGGTATCGACGTGAAGGAAGTG
GACCCTACCGGTCACTCCTTCGTGCTGGTCACCTCCCTGGGCCTGACCTACGACGGCATGCTGTCCGACGTGC
AGTCCATGCCCAAGACCGGTATCCTGATCCTCATCCTGTCCATCATCTTCATCGAGGGCTACTGCACTCCTGAG
GAAGTGATCTGGGAGGCTCTGAACATGATGGGCCTGTACGACGGAATGGAACACCTGATCTACGGCGAGCCC
CGCAAGCTGCTGACCCAGGACTGGGTGCAAGAGAACTACCTCGAGTACCGTCAGGTGCCCGGTTCCGACCCC
GCTCGTTACGAGTTCCTGTGGGGTCCCCGTGCTCACGCTGAGATCCGCAAGATGTCCGTGCTGAAGTTCCTGG
CTAAGGTCAACGGCTCCGACCCCCGTTCCTTCCCACTGTGGTACGAGGAAGCTCTGAAGGACGAGGAAGAGA
GGGCTCAGGACCGTATCGCTACCACCGACGACACCACCGCTATGGCTTCCGCTTCCTCTAGCGCTACCGGTTC
CTTCAGCTACCCCGAG
Protein Sequence(Seq ID No. 26):
MPRAPKRQPCMPEEQLQSQSETQGLEGAQAPLAVEEDASSSTSTSSSFPSSFPSSSSSSSSSCYPLIPSTPEEVSA
DDETPNPPQSAQIACSSPSVVASLPLDQSDEGSSSQKEESPSTLQVLPDSESLPRSEIDEKVTDLVGFLLFKYQMKE
PITKAEILESVIRNYEDHFPLLFSEASECMLLVFGIDVKEVDPTGHSFVLVTSLGLTYDGMLSDVQSMPKTGILILILSIVF
IEGYCTPEEVIWEALNMMGLYDGMEHLIYGEPRKLLTQDWVQENYLEYRQVPGSDPARYEFLWGPRAHAEIRKMSL
LKFLAKVNGSDPRSFPLWYEEALKDEEERAQDRIATTDDTTAMASASSSATGSFSYPE

| | | |
|---|---|---|
| ZNRD1 | Q9P1U0 | >P000875_TRN_TRNp2_ZNRD1_30834_Homo sapiens zinc ribbon domain |
| | | containg 1 transript variant |
| | | b_BC010898.1_AAH10898.1_Q9P1U0_0_0_381_0_378 |

Nucleotide Sequence(Seq ID No. 8):
ATGTCTGTCATGGACCTCGCCAATACTTGCTCCAGCTTTCAGTCGGACCTGGATTTCTGTTCAGATTGCGGCTC
GGTCCTGCCTCTGCCCGGGGCTCAGGATACGGTCACCTGTATTCGCTGTGGCTTCAACATCAACGTTCGGGAC
TTTGAGGGGAAGGTTGTGAAGACTTCGGTTGTGTTCCACCAACTGGGGACAGCCATGCCTATGTCGGTGGAGG
AAGGGCCTGAGTGCCAGGGACCTGTGGTTGACAGGCGCTGCCCTCGATGTGGTCATGAAGGAATGGCATACC
ACACCAGACAGATGCGTTCAGCCGATGAAGGGCAAACTGTCTTCTACACCTGTACCAACTGCAAGTTCCAGGA
GAAGGAAGACTCT
Protein Sequence(Seq ID No. 27):
MSVMDLANTCSSFQSDLDFCSDCGSVLPLPGAQDTVTCIRCGFNINVRDFEGKVVKTSVVFHQLGTAMPMSVEEGP
ECQGPVVDRRCPRCGHEGMAYHTRQMRSADEGQTVFYTCTNCKFQEKEDS

| | | |
|---|---|---|
| MAP2K5 | Q1363 | >P000081_KIN96_KIN_MAP2K5_5607_Homo sapiens mitogen-activated |
| | | protein kinase kinase 5, transcript variant |
| | | A_BC008838.2_AAH08838.1_Q13163_4111679_0_1347_0_1344 |

Nucleotide Sequence(Seq ID No. 9):
ATGCTGTGGCTAGCCCTTGGCCCCTTTCCTGCCATGGAGAACCAGGTGCTGGTAATTCGCATCAAGATCCCAAA
TAGTGGCGCGGTGGACTGGACAGTGCACTCCGGGCCGCAGTTACTCTTCAGGGATGTGCTGGATGTGATAGG
CCAGGTTCTGCCTGAAGCAACAACTAGAGCATTTGAATATGAAGATGAAGATGGTGATCGAATTACAGTGAGAA
GTGATGAGGAAATGAAGGCAATGCTGTCATATTATTATTCCACAGTAATGGAACAGCAAGTAAATGGACAGTTAA
TAGAGCCTCTGCAGATATTTCCAAGAGCCTGCAAGCCTCCTGGGGAACGGAACATACATGGCCTGAAGGTGAA
TACTCGGGCCGGACCCTCTCAACACAGCAGCCCAGCAGTCTCAGATTCACTTCCAAGCAATAGCTTAAAGAAGT
CTTCTGCTGAACTGAAAAAAATACTAGCCAATGGCCAGATGAATGAACAAGACATACGATATCGGGACACTCTT
GGTCATGGCAACGGAGGCACAGTCTACAAAGCATATCATGTCCCGAGTGGGAAAATATTAGCTGTAAAGGTCAT
ACTACTAGATATTACACTGGAACTTCAGAAGCAAATTATGTCTGAATTGGAAATTCTTTATAAGTGCGATTCATCA
TATATCATTGGATTTTATGGAGCATTTTTTGTAGAAAACAGGATTTCAATATGTACAGAATTCATGGATGGGGGAT
CTTTGGATGTATATAGGAAAATGCCAGAACATGTCCTTGGAAGAATTGCAGTAGCAGTTGTTAAAGGCCTTACTT
ATTGTGGAGTTTAAAGATTTTACATAGAGACGTGAAGCCCTCCAATATGCTAGTAAACACAAGAGGACAGGTTA
AGCTGTGTGATTTTGGAGTTAGCACTCAGCTGGTGAATTCTATAGCCAAGACGTATGTTGGAACAAATGCTTATA
TGGCGCCTGAAAGGATTTCAGGGGAGCAGTATGGAATTCATTCTGATGTCTGGAGCTTAGGAATCTCTTTTATG
GAGCTTGCTCTTGGGAGGTTTCCATATCCTCAGATTCAGAAAAACCAGGGATCTTTAATGCCTCTCCAGCTTCTG
CAGTGCATTGTTGATGAGGATTCGCCCGTCCTTCCAGTTGGAGAGTTCTCGGAGCCATTTGTACATTTCATCAG
TCAGTGTATGCGAAAACAGCCAAAAGAAAGGCCAGCACCTGAAGAATTGATGGGCCACCCGTTCATCGTGCAG
TTCAATGATGGAAATGCCGCCGTGGTGTCCATGTGGGTGTGCCGGGCGCTGGAGGAGAGGCGGAGCCAGCAG
GGGCCCCCG
Protein Sequence(Seq ID No. 28):
MLWLALGPFPAMENQVLVIRIKIPNSGAVDWTVHSGPQLLFRDVLDVIGQVLPEATTTAFEYEDEDGDRITVRSDEEM
KAMLSYYYSTVMEQQVNGQLIEPLQIFPRACKPPGERNIHGLKVNTRAGPSQHSSPAVSDSLPSNSLKKSSAELKKIL
ANGQMNEQDIRYRDTLGHGNGGTVYKAYHVPSGKILAVKVILLDITLELQKQIMSELEILYKCDSSYIIGFYGAFFVENR
ISICTEFMDGGSLDVYRKMPEHVLGRIAVAVVKGLTYLWSLKILHRDVKPSNMLVNTRGQVKLCDFGVSTQLVNSIAK
TYVGTNAYMAPERISGEQYGIHSDVWSLGISFMELALGRFPYPQIQKNQGSLMPLQLLQCIVDEDSPVLPVGEFSEPF
VHFITQCMRKQPKERPAPEELMGHPFIVQFNDGNAAVVSMWVCRALEERRSQQGPP

| | | |
|---|---|---|
| MAGEA4 | P43358 | >P001216_CAG_CAGp1_MAGEA4_3103_Homo sapiens melanoma antigen |
| | | family A 4_BC017723.1_AAH17723.1_P43358_0_0_054_0_951 |

Nucleotide Sequence(Seq ID No. 10):
ATGTCTTCTGAGCAGAAGAGTCAGCACTGCAAGCCTGAGGAAGGCGTTGAGGCCCAAGAAGAGGCCCTGGGC
CTGGTGGGTGCACAGGCTCCTACTACTGAGGAGCAGGAGGCTGCTGTCTCCTCCTCCTCTCCTCTGGTCCCTG
GCACCCTGGAGGAAGTGCCTGCTGCTGAGTCAGCAGGTCCTCCCCAGAGTCCTCAGGGAGCCTCTGCCTTAC
CCACTACCATCAGCTTCACTTGCTGGAGGCAAGCCAATGAGGGTTCCAGCAGCCAAGAAGAGGAGGGGGCAA
GCACCTCGCCTGACGCAGAGTCCTTGTTCCGAGAAGCACTCAGTAACAAGGTGGATGAGTTGGCTCATTTTCTG
CTCCGCAAGTATCGAGCCAAGGAGCTGGTCACAAAGGCAGAAATGCTGGAGAGAGTCATCAAAAATTACAAGC
GCTGCTTTCCTGTGATCTTCGGCAAAGCCTCCGAGTCCCTGAAGATGATCTTTGGCATTGACGTGAAGGAAGTG
GACCCCACCAGCAACACCTACACCCTTGTCACCTGCCTGGGCCTTTCCTATGATGGCCTGCTGGGTAATAATCA
GATCTTTCCCAAGACAGGCCTTCTGATAATCGTCCTGGGCACAATTGCAATGGAGGGCGACAGCGCCTCTGAG
GAGGAAATCTGGGAGGAGCTGGGTGTGATGGGGGTGTATGATGGGAGGGAGCACACTGTCTATGGGGAGCCC
AGGAAACTGCTCACCCAAGATTGGGTGCAGGAAAACTACCTGGAGTACCGGCAGGTACCCGGCAGTAATCCTG
CGCGCTATGAGTTCCTGTGGGGTCCAAGGGCTCTGGCTGAAACCAGCTATGTGAAAGTCCTGGAGCATGTGGT
CAGGGTCAATGCAAGAGTTCGCATTGCCTACCCATCCCTGCGTGAAGCAGCTTTGTTAGAGGAGGAAGAGGGA
GTC
Protein Sequence(Seq ID No. 29):
MSSEQKSQHCKPEEGVEAQEEALGLVGAQAPTTEEQEAAVSSSSPLVPGTLEEVPAAESAGPPQSPQGASALPTTI
SFTCWRGPNEGSSSQEEEGPSTSPDAESLFREALSNKVDELAHFLLRKYRAKELVTKAEMLERVIKNYKRCFPVIFG
KASESLKMIFGIDVKEVDPASNTYTLVTCLGLSYDGLLGNNQIFPKTGLLIIVLGTIAMEGDSASEEEIWEELGVMGVYD
GREHTVYGEPRKLLTQDWVQENYLEYRQVPGSNPARYEFLWGPRALAETSYVKVLEHVVRVNARVRIAYPSLREAA
LLEEEEGV

| | | |
|---|---|---|
| STAT1 | P42224 | >P000068_KIN96_KIN_STAT1_6772_Homo sapiens signal trasducer and |
| | | activator of transcription 1 91 kDa transcript |
| | | varian_BC002704.2_AAH02704.1_P42224_93992.15_0_2139_0_2136 |

Nucleotide Sequence(Seq ID No. 11):
ATGTCTCAGTGGTACGAACTTCAGCAGCTTGACTCAAAATTCCTGGAGCAGGTTCACCAGCTTTATGATGACAG
TTTTCCCATGGAAATCAGACAGTACCTGGCAGAGTGGTTAGAAAAGCAAGACTGGGAGCACGCTGCCAATGAT
GTTTCATTTGCCACCATCCGTTTTCATGACCTCCTGTCACAGCTGGATGATCAATATAGTCGCTTTTCTTTGGAG
AATAACTTCTTGCTACAGCATAACATAAGGAAAAGCAAGCGTAATCTTCAGGATAATTTTCAGGAAGACCCAATC
CAGATGTCTATGATCATTTACAGCTGTCTGAAGGAAGAAAGGAAAATTCTGGAAAACGCCCAGAGATTTAATCAG
GCTCAGTCGGGGAATATTCAGAGCACAGTGATGTTAGACAAACAGAAAGAGCTTGACAGTAAAGTCAGAAATGT
GAAGGACAAGGTTATGTGTATAGAGCATGAAATCAAGAGCCTGGAAGATTTACAAGATGAATATGACTTCAAATG
CAAAACCTTGCAGAACAGAGAACACGAGACCAATGGTGTGGCAAAGAGTGATCAGAAACAAGAACAGCTGTTA
CTCAAGAAGATGTATTTAATGCTTGACAATAAGAGAAAGGAAGTAGTTCACAAAATAATAGAGTTGCTGAATGTC
ACTGAACTTACCCAGAATGCCCTGATTAATGATGAACTAGTGGAGTGGAAGCGGAGACAGCAGAGCGCCTGTA
TTGGGGGGCCGCCCAATGCTTGCTTGGATCAGCTGCAGAACTGGTTCACTATAGTTGCGGAGAGTCTGCAGCA
AGTTCGGCAGCAGCTTAAAAAGTTGGAGGAATTGGAACAGAAATACACCTACGAACATGACCCTATCACAAAAA
ACAAACAAGTGTTATGGGACCGCACCTTCAGTCTTTTCCAGCAGCTCATTCAGAGGTCGTTTGTGGTGGAAAGA
CAGCCCTGCATGCCAACGCACCCTCAGAGGCCGCTGGTCTTGAAGACAGGGGTCCAGTTCACTGTGAAGTTGA
GACTGTTGGTGAAATTGCAAGAGCTGAATTATAATTTGAAAGTCAAAGTCTTATTTGATAAAGATGTGAATGAGA
GAAATACAGTAAAAGGATTTAGGAAGTTCAACATTTTGGGCACGCACACAAAAGTGATGAACATGGAGGAGTCC
ACCAATGGCAGTCTGGCGGCTGAATTTCGGCACCTGCAATTGAAAGAACAGAAAAATGCTGGCACCAGAACGA
ATGAGGGTCCTCTCATCGTTACTGAAGAGCTTCACTCCCTTAGTTTTGAAACCCAATTGTGCCAGCCTGGTTTG
GTAATTGACCTCGAGACGACCTCTCTGCCCGTTGTGGTGATCTCCAACGTCAGCCAGCTCCCGAGCGGTTGGG
CCTCCATCCTTTGGTACAACATGCTGGTGGCGGAACCCAGGAATCTGTCCTTCTTCCTGACTCCACCATGTGCA
CGATGGGCTCAGCTTTCAGAAGTGCTGAGTTGGCAGTTTTCTTCTGTCACCAAAAGAGGTCTCAATGTGGACCA
GCTGAACATGTTGGGAGAGAAGCTTCTTGGTCCTAACGCCAGCCCCGATGGTCTCATTCCGTGGACGAGGTTT
TGTAAGGAAAATATAAATGATAAAAATTTTCCCTTCTGGCTTTGGATTGAAAGCATCCTAGAACTCATTAAAAAAC
ACCTGCTCCCTCTCTGGAATGATGGGTGCATCATGGGCTTCATCAGCAAGGAGCGAGAGCGTGCCCTGTTGAA
GGACCAGCAGCCGGGGACCTTCCTGCTGCGGTTCAGTGAGAGCTCCCGGGAAGGGGCCATCACATTCACATG
GGTGGAGCGGTCCCAGAACGGAGGCGAACCTGACTTCCATGCGGTTGAACCCTACACGAAGAAAGAACTTTCT
GCTGTTACTTTCCCTGACATCATTCGCAATTACAAAGTCATGGCTGCTGAGAATATTCCTGAGAATCCCCTGAAG
TATCTGTATCCAAATATTGACAAAGACCATGCCTTTGGAAAGTATTACTCCAGGCCAAAGGAAGCACCAGAGCC
AATGGAACTTGATGGCCCTAAAGGAACTGGATATATCAAGACTGAGTTGATTTCTGTGTCTGAAGTG
Protein Sequence(Seq ID No. 30):
MSQWYELQQLDSKFLEQVHQLYDDSFPMEIRQYLAQWLEKQDWEHAANDVSFATIRFHDLLSQLDDQYSRFSLEN
NFLLQHNIRKSKRNLQDNFQEDPIQMSMIIYSCLKEERKILENAQRFNQAQSGNIQSTVMLDKQKELDSKVRNVKDKV
MCIEHEIKSLEDLQDEYDFKCKTLQNREHETNGVAKSDQKQEQLLLKKMYLMLDNKRKEVVHKIIELLNVTELTQNALI
NDELVEWKRRQQSACIGGPPNACLDQLQNWFTIVAESLQQVRQQLKKLEELEQKYTYEHDPITKNKQVLWDRTFSL
FQQLIQSSFVVERQPCMPTHPQRPLVKTGVQFTVKLRLLVKLQELNYNLKVKVLFDKDVNERNTVKGFRKFNILGTH
TKVMNMEESTNGSLAAEFRHLQLKEQKNAGTRTNEGPLITEELHSLSFETQLCQPGLVIDLETTSLPVVVISNVSQL
PSGWASILWYNMLVAEPRNLSFFLTPPCARWAQLSEVLSWQFSSVTKRGLNVDQLNMLGEKLLGPNASPDGLIPWT
RFCKENINDKNFPFWLWIESILELIKKHLLPLWNDGCIMGFISKERERALLKDQQPGTFLLRFSESSREGAITFTWVER
SQNGGEPDFHAVEPYTKKELSAVTFPDIIRNYKVMAAENIPENPLKYLYPNIDKDHAFGKYYSRPKEAPEPMELDGPK
GTGYIKTELISVSEV

| | | |
|---|---|---|
| CT47A1 | Q5JQC4 | >P003099_Q211_Q211_tube_CT47A1_728096_0_NM_001080146.1_0_Q5JQC4_0_Insert |
| | | sequence is gene optimized by GeneArt_0_0_0 |

Nucleotide Sequence(Seq ID No. 12):
ATGTCCGCTACCGGCGACCGTCACCCTACCCAGGGCGACCAAGAGGCTCCCGTGTCCCAAGAGGGTGCTCAG
GCTGAGGCTGCTGGTGCTGGCAACCAGGAAGGTGGCGACTCCGGTCCCGACTCCTCCGACGTGGTGCCTGCT
GCTGAGGTCGTGGGTGTCGCTGGTCCTGTCGAGGGCCTGGGAGAGGAAGAGGGCGAGCAGGCTGCTGGCCT
GGCTGCTGTGCGTCGTGGTGGTTCCGCTGAAGAGGACTCCGACATCGGTCCCGCTACCGAGGAAGAGGAAGA
AGAAGAGGGCAACGAGGCTGCTAACTTCGACCTGGCTGTGGTGGCTCGTCGTTACCCCGCTTCCGGTATCCAC
TTCGTGCTGCTGGACATGGTGCACTCCCTGCTGCACCGTCTGTCCCACAACGACCACATCCTGATCGAGAACC
GTCAGCTGTCCCGTCTGATGGTCGGACCCCACGCTGCTGCTCGTAACCTGTGGGGCAACCTGCCCCCCCTGC
TGCTGCCTCAACGTCTGGGAGCTGGTGCTGCTGCTAGGGCTGGCGAGGGACTGGGCCTGATCCAAGAGGCTG
CTTCCGTGCCCGAGCCCGCTGTGCCTGCTGACTTGGCTGAGATGGCCCGCGAGCCTGCTGAGGAAGCTGCTG
AAGAGAAGCTGTCCGAGGAAGCCACCGAGGAACCCGACGCTGAGGAACCAGCTACTGAGGAACCCACCGCTC
AAGAGAAGCTGTCCGAGGAAGCCACCGAGGAACCCGACGCTGAGGAACCAGCTACTGAGGAACCCACCGCTC
CTGGCGAGGAAGAGAAGGAACAAGAAAAGGAAAAGGACGCCGAGAACAAGGTCAAGAACTCCAAGGGCACC
Protein Sequence(Seq ID No. 31):
MSATGDRHPTQGDQEAPVSQEGAQAEAAGAGNQEGGDSGPDSSDVVPAAEVVGVAGPVEGLGEEEGEQAAGLA
AVPRGGSAEEDSDIGPATEEEEEEEGNEAANFDLAVVARRYPASGIHFVLLDMVHSLLHRLSHNDHILIENRQLSRLM
VGPHAAARNLWGNLPPLLLPQRLGAGAAARAGEGLGLIQEAASVPEPAVPADLAEMAREPAEEAAEEKLSEEATEE
PDAEEPATEEPTAQEATAPEEVTKSQPEKWDEEAQDAAGEEEKEQEKEKDAENKVKNSKGT

| | | |
|---|---|---|
| IGF2BP3 | O00425 | >P003224_Q311_Q311_tube_IGF2BP3_10643_0_NM_006547.2_0_O00425_0_Insert |
| | | sequence is gene optimized by GeneArt_0_0_0 |

Nucleotide Sequence(Seq Id No. 13):
ATGAACAAGCTGTACATCGGCAACCTGTCCGAGAACGCTGCTCCCTCCGACCTCGAGTCCATCTTCAAGGACG
CTAAGATCCCCGTGTCCGGACCCTTCCTGGTCAAGACCGGTTACGCTTTCGTGGACTGCCCCGACGAGTCCTG
GGCTCTGAAGGCTATCGAGGCTCTGTCCGGCAAGATCGAGCTGCACGGCAAGCCCATCGAGGTCGAGCACTC
CGTGCCCAAGCGTCAGCGTATCCGCAAGCTGCAGATCCGTAACATCCCCCCACACCTCCAGTGGGAGGTGCT
GGACTCCCTGCTGGTGCAGTACGGTGTCGTCGAGTCCTGCGAGCAAGTGAACACCGACTCCGAGACTGCTGT
GGTCAACGTGAGCTACTCCTCCAAGGACCAGGCTCGTCAGGCTCTGGACAAGCTGAACGGTTTCCAGCTCGAG
AACTTCACCCTGAAGGTGGCCTACATCCCCGACGAGATGGCTGCTCAGCAGAACCCCCTGCAGCAGCCCCGT
GGTCGTCGTGGACTGGGACAGCGTGGTAGCTCCCGTCAGGGTTCCCCCGGTTCCGTGTCCAAGCAGAAGCCC
TGCGACCTGCCCCTGCGTCTGCTGGTGCCTACCCAGTTCGTGGGTGCTATCATCGGCAAGGAAGGTGCTACCA
TCCGCAACATCACCAAGCAGACCCAGTCCAAGATCGACGTCCACCGCAAGGAAAACGCTGGCGCTGCTGAGAA
GTCCATCACCATCCTGTCCACCCCCGAGGGCACCTCCGCTGCTTGCAAGTCCATCCTCGAGATCATGCACAAG
GAAGCCCAGGACATCAAGTTCACCGAGGAAATCCCCCTGAAGATCCTGGCTCACAACAACTTCGTGGGTCGTC
TGATCGGAAAGGAAGGCCGTAACCTGAAGAAGATCGAGCAGGACACCGACACCAAGATCACCATCTCCCCACT
GCAAGAGCTGACCCTGTACAACCCCGAGCGTACCATCACCGTGAAGGGCAACGTGGAAACCTGCGCTAAGGC
TGAAGAGGAAATCATGAAGAAGATCCGCGAGTCCTACGAGAACGATATCGCTTCCATGAACCTGCAGGCTCAC
CTGATCCCCGGCCTGAACCTGAACGCTCTGGGCCTGTTCCCCCCTACCTCCGGCATGCCTCCTCCCACCTCTG
GTCCCCCCTCCGCTATGACCCCCCCATACCCCCAGTTCGAGCAGTCCGAGACTGAGACTGTGCACCTGTTCAT
CCCCGCTCTGTCCGTCGGTGCCATCATCGGAAAGCAGGGCCAGCACATCAAGCAGCTGTCCCGTTTCGCTGGT
GCTTCCATCAAGATCGCTCCCGCTGAGGCTCCCGACGCTAAGGTCCGCATGGTCATCATCACCGGTCCCCCCG
AGGCTCAGTTCAAGGCTCAGGGTCGTATCTACGGCAAGATCAAGGAAGAGAACTTCGTCAGCCCCAAGGAAGA
AGTGAAGCTCGAGGCTCACATCCGTGTGCCATCCTTCGCTGCTGGTCGTGTTATCGGCAAGGGTGGCAAGACC
GTGAACGAGCTGCAGAACCTGTCCTCCGCTGAGGTGGTGGTGCCCCGTGACCAGACCCCTGACGAGAACGAC
CAGGTGGTGGTCAAGATCACCGGTCACTTCTACGCTTGCCAGGTGGCCCAGCGCAAGATCCAAGAGATCCTGA
CCCAAGTGAAGCAGCACCAGCAGCAGAAGGCTCTGCAGTCCGGTCCCCCTCAGTCCCGTCGCAAG
Protein Sequence(Seq ID No. 32):
MNKLYIGNLSENAAPSDLESIFKDAKIPVSGPFLVKTGVAFVDCPDESWALKAIEALSGKIELHGKPIEVEHSVPKRQRI
RKLQIRNIPPHLQWEVLDSLLVQYGVVESCEQVNTDSETAVVNVTYSSKDQARQALDKLNGFQLENFTLKVAYIPDE
MAAQQNPLQQPRGRRGLGQRGSSRQGSPGSVSKQKPCDLPLRLLVPTQFVGAIIGKEGATIRNITKQTQSKIDVHRK
ENAGAAEKSITILSTPEGTSAACKSILEIMHKEAQDIKFTEEIPLKILAHNNFVGRLIGKEGRNLKKIEQDTDTKITISPLQE
LTLYNPERTITVKGNVETCAKAEEEIMKKIRESYENDIASMNLQAHLIPGLNLNALGLFPPTSGMPPPTSGPPSAMTPP
YPQFEQSETETVHLFIPALSVGAIIGKQGQHIKQLSRFAGASIKIAPAEAPDAKVRMVIITGPPEAQFKAQGRIYGKIKEE
NFVSPKEEVKLEAHIRVPSFAAGRVIGKGGKTVNELQNLSSAEVVVPRDQTPDENDQVVVKITGHFYACQVAQRKIQ
EILTQVKQHQQQKALQSGPPQSRRK

| | | |
|---|---|---|
| CTAG2 | O75638 | >P001440_CAG_CAGp2_CTAG2_30848_Homo sapiens cancer/testis antigen |
| | | 2 transcript variant 2_BC002833.2_AAH02833.1_O75638_0_0_633_0_630 |

Nucleotide Sequence(Seq ID No. 14):
ATGCAGGCCGAAGGCCGGGGCACAGGGGGTTCGACGGGCGATGCTGATGGCCCAGGAGGCCCTGGCATTCC
TGATGGCCCAGGGGGCAATGCTGGCGGCCCAGGAGAGGCGGGTGCCACGGGCGGCAGAGGTCCCCGGGGC
GCAGGGGCAGCAAGGGCCTCGGGGCCGAGAGGAGGCGCCCCGCGGGGTCCGCATGGCGGTGCCGCTTCTG
CGCAGGATGGAAGGTGCCCCTGCGGGGCCAGGAGGCCGGACAGCCGCCTGCTTGAGTTGCACATCACGATG
CCTTTCTCGTCGCCCATGGAAGCGGAGCTGGTCCGCAGGATCCTGTCCCGGGATGCCGCACCGCTCCCCCGA
CCAGGGGCGGTTCTGAAGGACTTCACCGTGTCCGGCAACCTACTGTTTATGTCAGTTCGGGACCAGGACAGGG
AAGGCGCTGGGCGGATGAGGGTGGTGGGTTGGGGGCTGGGATCCGCCTCCCCGGAGGGGCAGAAAGCTAGA
GATCTCAGAACACCCAAACACAAGGTCTCAGAACAGAGACCTGGTACACCAGGCCCGCCGCCACCCGAGGGA
GCCCAGGGAGATGGGTGCAGAGGTGTCGCCTTTAATGTGATGTTCTCTGCCCCTCACATT
Protein Sequence(Seq ID No. 33):
MQAEGQGTGGSTGDADGPGGPGIPDGPGGNAGGPGEAGATGGRGPRGAGAARASGPRGGAPRGPHGGAASAQ
DGRCPCGARRPDSRLLQLHITMPFSSPMEAELVRRILSRDAAPLPRPGAVLKDFTVSGNLLFMSVRDQDREGAGRM
RVVGWGLGSASPEGQKARDLRTPKHKVSEQRPGTPGPPPPEGAQGDGCRGVAFNVMFSAPHI

| | | |
|---|---|---|
| RAD23B | P54727 | >P00309_SIG_SIG1-1_RAD23B_5887_Homo sapiens RAD23 homolog B (S. |
| | | cerevisiae)_BC020973.2_AAH20973.1_P54727_54173_0_1230_0_1227 |

Nucleotide Sequence(Seq ID No. 15):
ATGCAGGTCACCCTGAAGACCCTCCAGCAGCAGACCTTCAAGATAGACATTGACCCCGAGGAGACGGTGAAAG
CACTGAAAGAGAAGATTGAATCTGAAAAGGGGAAAGATGCCTTTCCAGTAGCAGGTCAAAAATTAATTTATGCA
GGCAAAATCCTCAATGATGATACTGCTCTCAAAGAATATAAAATTGATGAGAAAAACTTTGTGGTGGTTATGGTG
ACCAAACCCAAAGCAGTGTCCACACCAGCACCAGCTACAACTCAGCAGTCAGCTCCTGCCAGCACTACAGCAG
TTACTTCCTCCACCACCACAACTGTGGCTCAGGCTCCAACCCCTGTCCCTGCCTTGGCCCCCACTTCCACACCT
GCATCCATCACTCCAGCATCAGCGACAGCATCTTCTGAACCTGCACCTGCTAGTGCAGCTAAACAAGAGAAGCC
TGCAGAAAAGCCAGCACAGACACCAGTGGCTACTAGCCCAACAGCAACTGACAGTACATCGGGTGATTCTTCT
CGGTCAAACCTTTTTGAAGATGCAACGAGTGCACTTGTGACGGGTCAGTCTTACGAGAATATGGTAACTGAGAT
CATGTCAATGGGCTATGAACGAGAGCAAGTAATTGCAGCCCTGAGAGCCAGTTTCAACAACCCTGACAGAGCA
GTGGAGTATCTTTTAATGGGAATCCCTGGAGATAGAGAAAGTCAGGCTGTGGTTGACCCCCCTCAAGCAGCTA
GTACTGGGGTTCCTCAGTCTTCAGCAGTGGCTGCAGCTGCAGCAACTACGACAGCAACAACTACAACAACAAG
TTCTGGAGGACATCCCCTTGAATTTTTACGGAATCAGCCTCAGTTTCAACAGATGAGACAAATTATTCAGCAGAA
TCCTTCCTTGCTTCCAGCGTTACTACAGCAGATAGGTCGAGAGAATCCTCAATTACTTCAGCAAATTAGCCAACA
CCAGGAGCATTTTATTCAGATGTTAAATGAACCAGTTCAAGAAGCTGGTGGTCAAGGAGGAGGAGGTGGAGGT
GGCAGTGGAGGAATTGCAGAAGCTGGAAGTGGTCATATGAACTACATTCAAGTAACACCTCAGGAAAAAGAAG
CTATAGAAAGGTTAAAGGCATTAGGATTTCCTGAAGGACTTGTGATACAAGCGTATTTTGCTTGTGAGAAGAATG
AGAATTTGGCTGCCAATTTTCTTCTACAGCAGAACTTTGATGAAGAT
Protein Sequence(Seq ID No. 34):
MQVTLKTLQQQTFKIDIDPEETVKALKEKIESEKGKDAFPVAGQKLIYAGKILNDDTALKEYKIDEKNFVVVMVTKPKAV
STPAPATTQQSAPASTTAVTSSTTTTVAQAPTPVPALAPTSTPASITPASATASSEPAPASAAKQEKPAEKPAETPVA
TSPTATDSTSGDSSRSNLFEDATSALVTGQSYENMVTEIMSMGYEREQVIAALRASFNNPDRAVEYLLMGIPGDRES
QAVVDPPQAASTGAPQSSAVAAAAATTTATTTTTSSGGHPLEFLRNQPQFQQMRQIIQQNPSLLPALLQQIGRENPQ
LLQQISQHQEHFIQMLNEPVQEAGGQGGGGGGGSGGIAEAGSGHMNYIQVTPQEKEAIERLKALGFPEGLVIQAYFA
CEKNENLAANFLLQQNFDED

| | | |
|---|---|---|
| FADD | Q13158 | >P000417_SIG_SIG1-2_FADD_8772_Homo sapiens Fas (TNFRSF6)- |
| | | associated via death |
| | | domain_BC000334.2_AAH00334.1_Q13158_34265.45_0_627_0_624 |

Nucleotide Sequence(Seq ID No. 16):
ATGGACCCGTTCCTGGTGCTGCTGCACTCGGTGTCGTCCAGCCTGTCGAGCAGCGAGCTGACCGAGCTCAAG
TTCCTATGCCTCGGGCGCGTGGGCAAGCGCAAGCTGGAGCGCGTGCAGAGCGGCCTAGACCTCTTCTCCATG
CTGCTGGAGCAGAACGACCTGGAGCCCGGGCACACCGAGCTCCTGCGCGAGCTGCTCGCCTCCCTGCGGCG
CCACGACCTGCTGCGGCGCGTCGACGACTTCGAGGCGGGGGCGGCGGCCGGGGCCGCGCCTGGGGAAGAA
GACCTGTGTGCAGCATTTAACGTCATATGTGATAATGTGGGGAAAGATTGGAGAAGGCTGGCTCGTCAGCTCAA
AGTCTCAGACACCAAGATCGACAGCATCCAGGACAGATACCCCCGCAACCTGACAGAGCGTGTGCGGGAGTC
ACTGAGAATCTGGAAGAACACAGAGAAGGAGAACGCAACAGTGGCCCACCTGGTGGGGGCTCTCAGGTCCTG
CCAGATGAACCTGGTGGCTGACCTGGTACAAGAGGTTCAGCAGGCCCGTGACCTCCAGAACAGGAGTGGGGC
CATGTCCCCGATGTCATGGAACTCAGACGCATCTACCTCCGAAGCGTCC
Protein Sequence(Seq ID No. 35):
MDPFLVLLHSVSSSLSSSELTELKFLCLGRVGKRKLERVQSGLDLFSMLLEQNDLEPGHTELLRELLASLRRHDLLRR
VDDFEAGAAAGAAPGEEDLCAAFNVICDNVGKDWRRLARQLKVSDTKIDSIEQRYPRNLTERVRESLRIWKNTEKEN
ATVAHLVGALRSCQMNLVADLVQEVQQARDLQNRSGAMSPMSWNSDASTSEAS

| | | |
|---|---|---|
| PTPN20A | Q4JDL3 | >P003090_Q211_Q211_tube)PTPN20A_653129_0_NM_001042387.1_0_Q4JDL3_0_Insert |
| | | sequence is gene optimized by GeneArt_0_0_0 |

Nucleotide Sequence(Seq ID No. 17):
ATGTCCTCCCCCCGTGACTTCCGTGCTGAGCCCGTGAACGACTACGAGGGCAACGACTCCGAGGCTGAGGAC
CTGAACTTCCGTGAAACCCTGCCCTCCAGCTCCCAAGAGAACACCCCCCGTTCCAAGGTGTTCGAGAACAAGG
TCAACTCCGAGAAGGTCAAGCTGTCCCTGCGCAACTTCCCCCACAACGATTACGAGGACGTGTTCGAGGAACC
CTCCGAGTCCGGTTCCGACCCCTCCATGTGGACCGCTCGTGGTCCCTTCCGTCGTGACCGTTGGTCCTCCGAG
GACGAGGAAGCTGCTGGACCCTCCCAGGCTCTGTCCCCCCTGCTGTCCGACACCCGCAAGATCGTGTCCGAG
GGCGAGCTGGACCAGCTGGCTCAGATCCGTGCCCTGATCTTCAACTTCCACGAGCAGACCGCTATCAAGGACT
GCCTGAAGATCCTCGAGGAAAAGACCGCTGCTTACGACATCATGCAAGAGTTCATGGCTCTCGAGCTGAAGAA
CCTGCCCGGCGAGTTCAACTCCGGCAACCAGCCCTCCAACCGCGAGAAGAACCGTTACCGTGACATCCTGCCT
TTCCAGCACCACGGTTACTCCGGTCCCAACGAGCGTACCACCTTCTGGCACGGTTCCAACGAGGGTGCTGTGT
CCCTGCTGCTGCGCTACTGCGCT
Protein Sequence(Seq ID No. 36):
MSSPRDFRAEPVNDYEGNDSEAEDLNFRETLPSSSQENTPRSKVFENKVNSEKVKLSLRNFPHND¥EDVFEEPSES
GSDPSMWTARGPFRRDRWSSEDEEAAGPSQALSPLLSDTRKIVSEGELDQLAQIRPLIFNFHEQTAIKDCLKILEEKT
AAYDIMQEFMALELKNLPGEFNSGNQPSNREKNRYRDILPYDSTRVPLGKSKDYINASYIRIVNCGEEYFYIATQGPLL
STIDDFWQMVLENNSNVIAMITREIEGGIIKCYHYWPISLKKPLELKHFRVFLENYQILQYFIIRMFQVVEKSTGTSHSVK
QLQFTKWPDHGTPASADSFIKYIRYARKSHLTGPMVVHCSAGIGRTGVFLCVDVVFCAIVKNCSFNIMDIVAQMREQ
RSGMVQTKEQYHFCYDIVLEVLRKLLTLD

| | | |
|---|---|---|
| TPM1 | P09493 | >P001454_CAG_CAGp2_TPM1_7168_Homo sapiens tropomyosin 1 |
| | | (alpha)_BC007433_2_AAH07433.1_P09493_0_0_855_0_852 |

Nucleotide Sequence(Seq ID No. 18):
ATGGACGCCATCAAGAAGAAGATGCAGATGCTGAAGCTCGACAAGGAGAACGCCTTGGATCGAGCTGAGCAG
GCGGAGGCCGACAAGAAGGCGGCGGAAGACAGGAGCAAGCAGCTGGAAGATGAGCTGGTGTCACTGCAAAA
GAAACTCAAGGGCACCGAAGATGAACTGGACAAATATTCTGAGGCTCTCAAAGATGCCCAGGAGAAGCTGGAG
CTGGCAGAGAAAAAGGCCACCGATGCTGAAGCCGACGTAGCTTCTCTGAACAGACGCATCCAGCTGGTTGAGG
AAGAGTTGGATCGTGCCCAGGAGCGTCTGGCAACAGCTTTGCAGAAGCTGGAGGAAGCTGAGAAGGCAGCAG
ATGAGAGTGAGAGAGGCATGAAAGTCATTGAGAGTCGAGCCCAAAAAGATGAAGAAAAAATGGAAATTCAGGA
GATCCAACTGAAAGAGGCAAAGCACATTGCTGAAGATGCCGACCGCAAATATGAAGAGGTGGCCCGTAAGCTG
GTCATCATTGAGAGCGACCTGGAACGTGCAGAGGAGCGGGCTGAGCTCTCAGAAGGCCAAGTCCGACAGCTG
GAAGAACAATTAAGAATAATGGATCAGACCTTGAAAGCATTAATGGCTGCAGAGGATAAGTACTCGCAGAAGGA
AGACAGATATGAGGAAGAGATCAAGGTCCTTTCCGACAAGCTGAAGGAGGCTGAGACTCGGGCTGAGTTTGCG
GAGAGGTCAGTAACTAAATTGGAGAAAAGCATTGATGACTTAGAAGACGAGCTGTACGCTCAGAAACTGAAGTA
CAAAGCCATCAGCGAGGAGCTGGACCACGCTCTCAACGATATGACTTCCATG
Protein Sequence(Seq ID No. 37):
MDAIKKKMQMLKLDKENALDRAEQAEADKKAAEDRSKQLEDELVSLQKKLKGTEDELDKYSEALKDAQEKLELAEKK
ATDAEADVASLNRRIQLVEEELDRAQERLATALQKLEEAEKAADESERGMKVIESRAQKDEEKMEIQEIQLKEAKHIA
EDADRKYEEVARKLVIIESDLERAEERAELSEGKCAELEEELKTVTNNLKSLEAQAEKYSQKEDRYEEEIKVLSDKLKE
AETRAEFAERSVTKLEKSIDDLEDELYAQKLKYKAISEELDHALNDMTSI

| | | |
|---|---|---|
| CTAG1A | P78358 | >P003024_Q211_Q211_tube_CTAG1A_246100_0_NM_139250.1_0_P78358_0_Insert |
| | | sequence is gene optimized by GeneArt_0_0_0 |

Nucleotide Sequence(Seq ID No. 19):
ATGCAGGCTGAGGGTCGTGGCACCGGTGGTTCCACTGGCGACGCTGACGGTCCCGGTGGTCCTGGTATCCCC
GACGGTCCTGGTGGCAACGCTGGTGGTCCAGGCGAGGCTGGTGCTACCGGTGGTCGTGGTCCTCGTGGTGCT
GGTGCTGCTCGTGCTTCCGGTCCAGGTGGTGGTGCTCCCCGTGGTCCTCACGGTGGTGCTGCTTCCGGCCTG
AACGGTTGCTGCCGTTGCGGTGCTCGCGGTCCCGAGTCCCGTCTGCTCGAGTTCTACCTGGCTATGCCCTTCG
CTACCCCTATGGAAGCTGAGCTGGCTCGTCGTTCCCTGGCTCAGGACGCTCCTCCTCTGCCCGTGCCCGGTGT
CCTGCTGAAGGAGTTCACTGTCTCCGGCAACATCCTGACCATCCGTCTGACCGCTGCTGACCACCGTCAGCTC
CAGCTGTCCATCTCCTCATGCCTGCAGCAGCTGTCCCTGCTGATGTGGATCACCCAGTGTTTCTTGCCCGTGTT
GGTGGCTCAGCCCCCCTCCGGTCAACGTCGT
Protein Sequence(Seq ID No. 38):
MQAEGRGTGGSTGDADGPGGPGIPDGPGGNAGGPGEAGATGGRGPRGAGAARASGPGGGAPRGPHGGAASGL
NGCCRCGARGPESRLLEFYLAMPFATPMEAELARRSLAQDAPPLPVPGVLLKEFTVSGNILTSRLTAADHRQLQLSIS
SCLQQLSLLMWITQCFLPVFLAQPPSGQRR

TABLE 6
Normalised RFU values for the 19 biomarkers

| Protein | CT47A1 | CTAG1A | CTAG2 | DDX43 | DDX53 | FADD | GAGE1 | GAGE2C |
|---|---|---|---|---|---|---|---|---|
| Control1_021608 | 1509.358 | 1665.573 | 1625.151 | 2051.253 | 2063.69 | 2342.416 | 1936.961 | 2921.207 |
| Control1_021611 | 744.8538 | 840.2482 | 689.2973 | 1011.785 | 1008.013 | 1106.472 | 1000.557 | 905.3507 |
| Control1_021630 | 2308.622 | 2320.635 | 3698.177 | 2686.878 | 2394.465 | 4457.038 | 2656.298 | 1767.186 |
| Control1_021631 | 1648.979 | 1653.373 | 2014.062 | 1947.228 | 1745.636 | 3154.698 | 1963.353 | 2408.739 |
| Control1_021642 | 1908.076 | 2463.134 | 2466.247 | 2669.508 | 2731.679 | 3765.574 | 2711.856 | 2790.404 |
| Control1_021643 | 903.8178 | 1280.729 | 1094.733 | 1480.856 | 1737.577 | 1901.206 | 1653.898 | 1536.486 |
| Control1_021650 | 2268.797 | 2064.734 | 3027.739 | 1922.742 | 1862.127 | 6656.163 | 1702.062 | 1538.051 |
| Control1_021660 | 1646.455 | 1641.704 | 2635.412 | 2104.055 | 2228.304 | 2471.875 | 2434.567 | 2913.973 |
| Control1_021661 | 1813.748 | 2232.638 | 1907.396 | 3502.799 | 3281.538 | 2910.518 | 3433.232 | 2851.365 |
| Control1_021663 | 7361.666 | 1747.892 | 1808.937 | 3356.196 | 2994.698 | 2869.315 | 2993.111 | 2762.496 |
| Control1_021674 | 1065.267 | 1391.603 | 1352.962 | 2290.515 | 2156.563 | 2278.898 | 2439.543 | 1912.377 |
| Control1_021679 | 1154.795 | 956.4944 | 1118.479 | 1632.494 | 1530.023 | 2318.788 | 1627.435 | 1436.882 |
| Control1_021680 | 3498.123 | 1930.224 | 1855.749 | 2352.687 | 2406.267 | 2802.602 | 2489.483 | 2315.313 |
| Control1_021681 | 8854.106 | 8411.776 | 8780.085 | 4507.529 | 18451.28 | 9194.071 | 4543.1 | 3534.392 |
| Control1_021682 | 1968.383 | 1557.664 | 1865.677 | 2790.045 | 2426.774 | 2398.539 | 2494.837 | 2171.401 |
| Control2_021005 | 19062.75 | 18286.29 | 21721.06 | 7277.07 | 7509.16 | 21723.39 | 8024.274 | 7810.164 |
| Control2_021007 | 3700.532 | 4218.712 | 3151.603 | 4712.313 | 3869.997 | 4623.076 | 2852.438 | 2929.432 |
| Control2_021016 | 2798.772 | 1907.347 | 2099.19 | 2252.848 | 2179.747 | 3351.127 | 2478.487 | 1937.812 |
| Control2_021017 | 1753.571 | 1402.848 | 1540.14 | 1802.829 | 1780.391 | 2588.142 | 1590.338 | 1678.784 |
| Control2_021025 | 1559 | 2078.736 | 1612.691 | 2155.411 | 2566.253 | 2697.4 | 2064.229 | 1783.645 |
| Control2_021037 | 1640.906 | 1349.049 | 1760.375 | 1917.732 | 2594.986 | 2383.889 | 1833.83 | 1568.244 |
| Control2_021038 | 1296.442 | 1068.401 | 1352.522 | 1510.341 | 1483.363 | 1730.292 | 1498.059 | 1215.978 |
| Control2_021045 | 1755.101 | 1821.33 | 1657.713 | 2107.653 | 2190.138 | 2525.574 | 2252.285 | 1949.365 |
| Control2_021046 | 2544.023 | 3285.646 | 3830.951 | 3493.117 | 4036.784 | 3754.021 | 2887.819 | 2281.996 |
| Control2_021401 | 1790.79 | 2078.056 | 1554.37 | 2146.542 | 2368.271 | 2731.86 | 2223.912 | 2136.282 |
| Control2_021405 | 1633.525 | 1266.149 | 1722.513 | 1767.528 | 1695.027 | 2204.463 | 1972.807 | 1588.648 |
| Control2_021406 | 1231.18 | 1052.587 | 1221.755 | 1492.635 | 1419.205 | 1617.316 | 1516.808 | 1242.477 |
| Control2_021419 | 1161.514 | 969.2148 | 1340.876 | 1298.33 | 1271.956 | 1791.68 | 1169.562 | 1080.648 |
| Control2_021420 | 1134.712 | 1149.875 | 1308.413 | 1473.83 | 1407.462 | 1798.174 | 1488.179 | 1345.406 |
| Control2_021423 | 2775.048 | 2484.158 | 3319.35 | 3998.832 | 3444.215 | 5014.536 | 2747.573 | 2749.234 |
| Control2_021426 | 1832.683 | 3149.643 | 3941.681 | 3331.141 | 3193.393 | 8540.402 | 2683.709 | 2773.037 |
| Control2_021430 | 1740.691 | 1262.074 | 1439.203 | 1784.948 | 1981.945 | 2212.359 | 1632.47 | 1614.165 |
| Control2_021436 | 1238.38 | 1622.209 | 1339.236 | 1766.58 | 1570.416 | 1970.871 | 1839.317 | 6397.377 |
| Control2_021451 | 2114.425 | 1537.93 | 2366.879 | 2916.57 | 2079.048 | 3845.347 | 2548.36 | 2707.548 |
| Control2_021453 | 1618.877 | 1772.711 | 1704.868 | 2010.795 | 1923.692 | 2306.765 | 1938.773 | 1756.808 |
| Control2_021454 | 1238.661 | 1215.827 | 1245.483 | 1523.407 | 1445.537 | 1949.649 | 1606.287 | 1406.954 |
| Control2_021455 | 2118.806 | 2056.799 | 2135.804 | 2460.721 | 2409.344 | 3278.959 | 2368.928 | 2369.251 |
| Control2_021463 | 3277.456 | 2954.683 | 6329.681 | 4028.11 | 3885.942 | 8013.651 | 3643.477 | 3340.58 |
| Control2_021470 | 1404.597 | 1243.895 | 1509.513 | 1683.949 | 1605.746 | 2041.225 | 1716.442 | 1589.304 |
| Control2_021477 | 3228.131 | 2485.288 | 2693.976 | 3065.942 | 3048.999 | 4125.412 | 2669.848 | 2729.497 |
| Control2_021478 | 2702.577 | 2203.357 | 3179.785 | 2097.886 | 1878.866 | 3515.833 | 1795.588 | 1763.884 |
| Control2_021484 | 1867.657 | 1752.908 | 1825.902 | 2021.439 | 1897.227 | 4221.542 | 2058.948 | 1997.38 |
| Control2_021494 | 1226.113 | 1187.936 | 1371.995 | 1461.011 | 1289.884 | 1730.896 | 1411.166 | 1264.948 |
| Control2_021495 | 7510.731 | 6547.905 | 8569.185 | 9460.011 | 24887.84 | 11979 | 7132.448 | 8605.022 |
| Control2_021497 | 2103.323 | 2325.289 | 2423.39 | 2140.501 | 1881.936 | 2612.635 | 1915.289 | 1828.612 |
| Control2_021801 | 1684.077 | 1614.954 | 1535.561 | 2231.253 | 1987.477 | 1996.131 | 2130.707 | 1799.308 |
| Control2_021802 | 1196.061 | 1161.766 | 1299.801 | 1585.175 | 1516.791 | 1907.229 | 1876.651 | 1209.1 |
| Control2_021804 | 1813.897 | 1627.858 | 1980.221 | 2420.979 | 2252.938 | 2496.24 | 2142.618 | 1722.867 |
| Control2_021805 | 1823.253 | 1975.388 | 1764.159 | 1876.648 | 2049.388 | 2313.581 | 1586.62 | 1558.38 |
| Control2_021806 | 3227.12 | 2526.679 | 3146.424 | 3098.552 | 2464.066 | 3832.353 | 2866.158 | 2476.056 |
| Control2_021809 | 7768.215 | 1240.79 | 1653.211 | 1662.744 | 1754.941 | 2201.225 | 1664.758 | 1504.47 |
| Control2_021810 | 2441.267 | 2074.878 | 2048.36 | 2517.456 | 2467.174 | 2497.116 | 2520.784 | 2145.236 |
| Control2_021811 | 2362.442 | 1943.874 | 2300.76 | 1417.942 | 1302.352 | 2023.503 | 1786.874 | 2726.91 |
| Control2_021812 | 3601.507 | 3159.063 | 3554.258 | 4238.192 | 3735.361 | 4730.045 | 3681.06 | 3574.706 |
| Control2_021818 | 1212.13 | 1505.043 | 1698.016 | 2248.258 | 17693.85 | 3775.838 | 1669.632 | 1509.442 |
| Control2_021822 | 1783.632 | 3716.23 | 1896.898 | 2328.595 | 2554.369 | 4283.265 | 2294.682 | 2177.366 |
| Control2_021823 | 1099.564 | 2210.086 | 1106.176 | 1579.562 | 2073.087 | 2047.938 | 1886.623 | 1732.564 |
| Control2_021824 | 5689.149 | 3643.473 | 5707.856 | 2441.248 | 1596.983 | 7271.611 | 2337.595 | 1735.312 |
| Control2_021825 | 1927.505 | 1370.127 | 1906.145 | 2845.125 | 2542.267 | 3858.186 | 2619.755 | 2365.264 |
| Control2_021826 | 2170.275 | 2212.367 | 2186.7 | 2585.956 | 3076.474 | 2347.794 | 2926.708 | 2668.387 |
| Control2_021829 | 727.9997 | 821.4169 | 966.9506 | 1252.757 | 1200.859 | 1308.101 | 1360.473 | 991.0163 |
| Control2_021831 | 1082.576 | 1191.876 | 1338.483 | 1541.967 | 1694.252 | 1659.618 | 1599.125 | 1224.445 |
| Control2_021834 | 2115.083 | 2133.577 | 2971.31 | 2478.802 | 2751.062 | 4492.736 | 2664.171 | 2371.209 |
| Control2_021835 | 1800.552 | 4143.1 | 2827.079 | 2165.51 | 1599.139 | 3511.991 | 1954.184 | 1569.674 |
| Control2_021836 | 1245.328 | 3282.619 | 1606.868 | 2021.895 | 2213.233 | 2169.312 | 1826.482 | 1525.452 |
| Control2_021837 | 1232.15 | 1843.519 | 1565.995 | 1737.465 | 1662.996 | 3514.607 | 1634.608 | 1499.075 |
| Control2_021839 | 1520.202 | 1957.838 | 1960.711 | 2279.169 | 2273.681 | 3209.642 | 2471.854 | 2103.487 |
| Control2_021840 | 1006.846 | 1466.336 | 1352.943 | 1436.497 | 1554.106 | 2073.867 | 1482.862 | 2043.637 |
| Control2_021844 | 1527.763 | 2143.553 | 2124.143 | 2583.149 | 2526.574 | 3471.964 | 2444.126 | 2209.262 |
| Control2_021845 | 1708.471 | 2597.062 | 3056.738 | 2764.51 | 2509.027 | 3627.698 | 2486.593 | 2214.397 |
| Control2_021848 | 1511.619 | 1337.722 | 1397.268 | 1647.631 | 1456.605 | 2385.061 | 1408.622 | 1224.608 |
| Control2_021849 | 1866.328 | 2832.416 | 4456.51 | 2224.86 | 2056.209 | 3537.863 | 2201.666 | 1893.812 |
| Control2_021850 | 2220.246 | 1058.777 | 1760.467 | 2172.435 | 1948.469 | 2902.667 | 2243.549 | 1943.425 |
| Control2_021851 | 3033.04 | 2883.127 | 3632.569 | 3669.286 | 3355.756 | 4199.051 | 3220.745 | 3010.425 |
| Control2_021852 | 1652.118 | 1463.437 | 1920.526 | 2158.32 | 1879.306 | 2640.924 | 2086.825 | 1879.865 |
| Control2_021853 | 891.6693 | 1094.034 | 1197.545 | 1478.783 | 1579.288 | 1847.533 | 1463.02 | 1369.585 |
| Control2_021855 | 1430.95 | 1762.848 | 1859.345 | 2189.602 | 2208.481 | 2695.143 | 2471.515 | 1924.412 |
| Control2_021861 | 1597.079 | 1676.007 | 1582.567 | 1887.047 | 1917.212 | 2577.912 | 2051.175 | 1694.966 |
| Control2_021862 | 1113.862 | 1470.806 | 1490.145 | 1769.074 | 1888.085 | 2226.298 | 1665.863 | 1505.797 |
| Control2_021864 | 1668.539 | 1449.478 | 1434.442 | 1329.608 | 1308.309 | 1871.11 | 1584.434 | 1139.643 |
| Control2_021866 | 2106.947 | 3537.804 | 2356.46 | 3515.985 | 3030.057 | 4083.057 | 3325.615 | 2975.695 |
| Control2_021869 | 3801.609 | 4345.063 | 4445.315 | 2569.386 | 2154.123 | 5376.007 | 1874.028 | 1627.357 |
| Control2_021870 | 1405.225 | 1733.58 | 1940.332 | 2278.261 | 2483.545 | 3020.657 | 2064.894 | 1919.133 |
| Control2_021872 | 1286.815 | 7318.886 | 2611.127 | 2152.928 | 2157.179 | 2850.372 | 2268.582 | 2110.246 |
| Control2_021874 | 1724.234 | 2365.071 | 2544.429 | 2717.166 | 2505.942 | 3590.685 | 2480.078 | 2400.147 |
| Control2_021875 | 1417.375 | 1366.168 | 1691.543 | 2298.582 | 2233.337 | 2976.039 | 2081.084 | 2989.963 |
| Control2_021876 | 1537.92 | 1520.497 | 1819.945 | 2074.254 | 2079.594 | 2127.166 | 1807.031 | 1649.114 |
| Control2_021877 | 2169.568 | 2520.335 | 2805.955 | 2901.827 | 2795.744 | 3439.995 | 2824.899 | 2605.987 |
| Control2_021882 | 1853.488 | 1463.824 | 2709.683 | 2630.134 | 2613.521 | 4889.535 | 2481.181 | 2151.083 |
| Control2_021884 | 1966.939 | 1262.075 | 1971.59 | 2222.155 | 2054.503 | 2464.76 | 2088.945 | 3236.594 |
| Control2_021885 | 2316.15 | 1641.508 | 2386.34 | 2210.695 | 2094.819 | 2590.088 | 2073.195 | 1878.722 |
| Control2_021887 | 1531.977 | 1308.395 | 1976.089 | 1951.533 | 1830.006 | 2269.807 | 1804.872 | 1627.947 |
| Control2_021888 | 1816.136 | 1727.809 | 2177.399 | 2355.914 | 2248.237 | 3075.7 | 2270.959 | 2016.192 |
| Control2_021889 | 1191.999 | 1224.372 | 1456.478 | 1671.608 | 1769.808 | 1729.559 | 1738.464 | 1479.342 |
| Control2_021893 | 961.8649 | 863.8429 | 1231.316 | 1339.618 | 1244.098 | 1399.984 | 1578.939 | 1009.998 |
| Control2_021896 | 1175.956 | 1869.874 | 1542.43 | 1767.732 | 1848.923 | 2319.642 | 1848.402 | 1313.737 |
| Control2_021898 | 1421.408 | 1274.498 | 1793.884 | 2191.324 | 2017.862 | 2316.975 | 2054.023 | 1780.855 |
| Control2_021899 | 1169.115 | 1018.919 | 1380.195 | 1747.41 | 1623.652 | 2066.902 | 1759.251 | 1608.307 |
| Control2_021900 | 1805.134 | 2272.669 | 2339.801 | 2877.322 | 2870.634 | 3650.904 | 3154.306 | 2943.415 |
| Control2_021962 | 1299.376 | 1469.512 | 1752.46 | 2072.822 | 2154.644 | 3048.746 | 1847.226 | 1801.589 |
| EarlyStg1_021633 | 888.7379 | 1083.956 | 1139.705 | 1745.988 | 1624.386 | 2126.853 | 1864.5 | 2451.019 |
| EarlyStg1_021651 | 1718.134 | 1869.381 | 2002.153 | 2829.701 | 2301.549 | 3393.112 | 2494.653 | 2191.697 |
| EarlyStg1_021654 | 1414.691 | 2009.296 | 1875.053 | 3674.002 | 3193.332 | 2959.501 | 3266.428 | 2816.123 |
| EarlyStg1_021655 | 2360.272 | 2071.366 | 2048.578 | 4079.875 | 3856.399 | 3233.548 | 3060.038 | 2967.291 |
| EarlyStg1_021662 | 1046.279 | 1038.759 | 1247.205 | 1608.22 | 1934.638 | 1675.178 | 1641.528 | 1478.774 |
| EarlyStg1_021675 | 2008.526 | 2211.421 | 2262.93 | 3836.686 | 3311.173 | 4348.88 | 3947.239 | 3216.422 |
| EarlyStg1_021678 | 809.411 | 1196.975 | 1171.397 | 1416.018 | 1513.61 | 1406.448 | 3201.611 | 3765.569 |
| EarlyStg2_021024 | 1666.611 | 2348.44 | 1861.542 | 1900.167 | 2175.75 | 2635.829 | 2138.762 | 1641.05 |
| EarlyStg2_021403 | 864.2702 | 908.2656 | 966.5297 | 1152.405 | 1152.189 | 1208.682 | 1132.829 | 999.4889 |
| EarlyStg2_021435 | 818.7927 | 946.9137 | 862.6614 | 1138.72 | 1062.796 | 1348.046 | 1297.819 | 1024.028 |
| EarlyStg2_021440 | 1115.634 | 1262.505 | 1256.625 | 1676.983 | 1456.852 | 1876.273 | 1804.083 | 1597.008 |
| EarlyStg2_021443 | 2209.834 | 2323.86 | 2268.553 | 4227.051 | 2746.796 | 3529.186 | 2838.406 | 2627.111 |
| EarlyStg2_021458 | 2764.343 | 2321.842 | 3096.833 | 2692.237 | 2468.356 | 3620.752 | 2475.345 | 2253.773 |
| EarlyStg2_021462 | 1168.597 | 1095.781 | 1259.984 | 1473.306 | 1355.856 | 1573.764 | 1735.746 | 1327.889 |
| EarlyStg2_021466 | 2917.855 | 2471.582 | 2442.846 | 3334.242 | 3444.158 | 3068.349 | 2739.576 | 2864.376 |
| EarlyStg2_021489 | 3814.376 | 64378.83 | 54002.38 | 1724.073 | 1619.1 | 2201.599 | 1688.194 | 1538.798 |
| EarlyStg2_021496 | 2140.219 | 2044.7 | 2620.907 | 2860.899 | 2714.917 | 3523.238 | 2716.084 | 4006.147 |
| EarlyStg2_021814 | 1434.233 | 1436.983 | 1273.89 | 1802.932 | 1544.033 | 1577.473 | 1621.802 | 1248.348 |
| EarlyStg2_021815 | 6157.471 | 3813.734 | 3852.406 | 8214.276 | 10197.87 | 7104.856 | 5543.301 | 6291.762 |
| EarlyStg2_021820 | 1272.23 | 1459.923 | 1404.584 | 2228.886 | 1796.769 | 2841.497 | 3885.665 | 4033.956 |
| EarlyStg2_021827 | 616.5434 | 786.2398 | 791.7878 | 977.2698 | 979.6347 | 1131.393 | 1511.574 | 838.4045 |
| EarlyStg2_021830 | 1350.571 | 1322.662 | 1347.479 | 1637.868 | 1574.216 | 1864.733 | 1802.094 | 1318.946 |
| EarlyStg2_021832 | 685.3966 | 768.7034 | 771.6687 | 907.4298 | 959.8832 | 881.5877 | 1484.217 | 1441.072 |
| EarlyStg2_021842 | 559.1504 | 728.8939 | 773.4969 | 886.8648 | 874.0025 | 1128.443 | 1246.985 | 812.1934 |
| EarlyStg2_021843 | 998.7775 | 1042.343 | 1037.048 | 1316.874 | 1545.713 | 1488.783 | 1464.504 | 1162.424 |
| EarlyStg2_021847 | 847.6904 | 834.0404 | 1065.834 | 1241.691 | 984.5962 | 1537.282 | 1765.055 | 1053.372 |
| EarlyStg2_021858 | 2884.206 | 3845.013 | 4219.314 | 3940.725 | 3779.591 | 4932.521 | 3850.321 | 3779.076 |
| EarlyStg2_021867 | 1027.782 | 3742.673 | 1421.976 | 1480.335 | 1415.318 | 1834.294 | 1702.586 | 1363.897 |
| EarlyStg2_021873 | 1991.314 | 64387.9 | 68263.43 | 2576.418 | 2450.933 | 3204.567 | 61022.98 | 61141.06 |
| EarlyStg2_021895 | 1309.615 | 1142.613 | 1833.176 | 1552.477 | 1668.209 | 2018.585 | 1748.757 | 1350.093 |
| EarlyStg2_021960 | 2893.513 | 3014.18 | 3981.726 | 31577.67 | 4138.143 | 6326.917 | 4832.018 | 7208.038 |
| LateStg1_021607 | 2233.818 | 33710.4 | 27509.89 | 2683.862 | 2431.354 | 5415.112 | 2263.816 | 2008.929 |
| LateStg1_021612 | 1077.574 | 1286.721 | 1463.395 | 1459.115 | 1434.265 | 1880.945 | 1247.632 | 1032.569 |
| LateStg1_021632 | 3286.639 | 1480.678 | 1291.98 | 1680.948 | 1599.405 | 1828.069 | 1593.324 | 1280.285 |
| LateStg1_021683 | 1332.092 | 920.1105 | 1136.96 | 1548.33 | 1181.713 | 1415.653 | 1581.494 | 1335.489 |
| LateStg1_021691 | 3844.224 | 3683.971 | 6199.383 | 3335.339 | 3994.33 | 8536.385 | 2753.94 | 2503.818 |
| LateStg1_021692 | 1632.176 | 7969.794 | 4384.263 | 2698.668 | 2282.849 | 2693.694 | 2417.603 | 2147.67 |
| LateStg1_021696 | 6599.412 | 5823.924 | 6186.081 | 5783.523 | 4699.548 | 7686.687 | 5192.874 | 4561.238 |
| LateStg1_021699 | 908.5383 | 943.1396 | 1345.248 | 1535.726 | 1361.538 | 2004.212 | 1679.826 | 1162.804 |
| LateStg2_021004 | 3221.85 | 4463.517 | 3620.611 | 3510.513 | 3849.087 | 5065.177 | 3227.483 | 3134.377 |
| LateStg2_021006 | 2339.927 | 2435.977 | 2422.967 | 3627.886 | 4482.165 | 3510.889 | 2848.815 | 2488.987 |
| LateStg2_021028 | 2707.792 | 4469.606 | 3026.737 | 4088.437 | 4885.905 | 4633.554 | 4514.939 | 5517.361 |
| LateStg2_021029 | 4950.915 | 4573.694 | 5365.522 | 5212.313 | 5258.751 | 7136.894 | 4720.304 | 4089.176 |
| LateStg2_021039 | 3522.816 | 3382.284 | 4025.145 | 4416.734 | 5475.508 | 4942.533 | 4098.692 | 4279.457 |
| LateStg2_021040 | 2002.684 | 1739.248 | 2184.697 | 2733.813 | 2964.582 | 2841.992 | 2364.913 | 2064.116 |
| LateStg2_021402 | 1361.212 | 1273.69 | 1436.17 | 30543.01 | 1661.542 | 2105.953 | 1792.681 | 1556.671 |
| LateStg2_021404 | 1825.759 | 1561.872 | 1859.06 | 2102.176 | 1937.207 | 2621.452 | 1884.312 | 1886.038 |
| LateStg2_021409 | 1821.798 | 1622.475 | 1888.661 | 2005.027 | 2042.316 | 2466.845 | 2172.961 | 1965.279 |
| LateStg2_021411 | 12831.44 | 2658.677 | 2981.266 | 3455.966 | 66480.3 | 3872.695 | 2669.909 | 2881 |
| LateStg2_021412 | 1375.199 | 1239.54 | 1325.421 | 1639.334 | 3807.727 | 1852.533 | 1571.277 | 1348.274 |
| LateStg2_021413 | 1771.981 | 1487.26 | 2007.343 | 1924.228 | 2049.404 | 2192.016 | 1768.964 | 1748.733 |
| LateStg2_021418 | 1312.756 | 1172.529 | 1327.484 | 1582.212 | 1511.383 | 2946.084 | 1518.947 | 1320.067 |
| LateStg2_021421 | 3372.893 | 1793.388 | 1865.367 | 1997.554 | 2003.966 | 2556.354 | 2058.865 | 1860.537 |
| LateStg2_021422 | 2876.725 | 2164.961 | 2960.132 | 2820.349 | 4247.47 | 3985.439 | 2012.289 | 1996.894 |
| LateStg2_021424 | 1510.005 | 1436.097 | 1764.94 | 2312.824 | 2095.89 | 2981.925 | 2034.561 | 2011.813 |
| LateStg2_021425 | 4155.837 | 3164.881 | 3549.289 | 4399.627 | 3495.884 | 69262.64 | 3726.524 | 3610.125 |
| LateStg2_021427 | 6471.712 | 5562.278 | 5414.813 | 2359.317 | 1931.492 | 5062.902 | 2487.975 | 2015.596 |
| LateStg2_021428 | 1315.405 | 1923.432 | 1325.323 | 1540.169 | 1567.625 | 1995.128 | 1862.058 | 1623.103 |
| LateStg2_021429 | 2123.084 | 2559.612 | 2236.494 | 3001.687 | 2607.627 | 3412.53 | 2292.985 | 2415.871 |
| LateStg2_021431 | 1761.144 | 1820.044 | 2118.511 | 2601.028 | 2288.129 | 3056.034 | 2245.269 | 3071.717 |
| LateStg2_021432 | 2360.041 | 2259.147 | 2011.696 | 2624.354 | 2347.857 | 3114.266 | 2267.014 | 2232.296 |
| LateStg2_021433 | 2535.536 | 2996.462 | 2903.991 | 3686.228 | 3635.17 | 44392.76 | 3044.162 | 3183.385 |
| LateStg2_021437 | 2480.932 | 2964.578 | 2577.313 | 3395.92 | 3330.783 | 3915.391 | 2871.597 | 3034.91 |
| LateStg2_021441 | 2178.753 | 3019.759 | 2484.399 | 3276.622 | 3424.187 | 4497.703 | 4627.948 | 5516.538 |
| LateStg2_021442 | 1402.243 | 1687.07 | 1595.617 | 2190.64 | 2035.534 | 2949.132 | 1854.978 | 1630.612 |
| LateStg2_021450 | 72225 | 4925.92 | 5586.278 | 7038.924 | 6264.657 | 9305.774 | 5407.448 | 6106.828 |
| LateStg2_021452 | 2351.882 | 2117.25 | 2515.784 | 2714.059 | 2709.585 | 3236.665 | 2472.468 | 2335.358 |
| LateStg2_021456 | 2281.465 | 1624.104 | 2069.136 | 2084.323 | 1906.072 | 2652.478 | 2118.836 | 1945.565 |
| LateStg2_021457 | 7111.74 | 5438.97 | 6081.31 | 7192.698 | 6515.937 | 8094.362 | 5778.938 | 6053.032 |
| LateStg2_021459 | 1662.911 | 1488.942 | 1709.95 | 5439.639 | 1756.361 | 2467.384 | 2151.735 | 1685.922 |
| LateStg2_021467 | 2871.091 | 2477.123 | 2697.42 | 3051.383 | 2669.106 | 4019.018 | 2084.887 | 2514.498 |
| LateStg2_021468 | 2239.595 | 2067.363 | 2455.974 | 2399.43 | 2260.627 | 3172.673 | 2334.31 | 2192.644 |
| LateStg2_021469 | 1134.769 | 1041.707 | 1124.96 | 1394.012 | 1423.355 | 1609.735 | 1685.225 | 1274.357 |
| LateStg2_021471 | 1883.788 | 1395.216 | 2140.146 | 2416.612 | 2918.033 | 2983.943 | 2138.6 | 2029.309 |
| LateStg2_021476 | 3258.855 | 2759.33 | 2993.915 | 3391.916 | 2972.729 | 4209.934 | 2853.457 | 3199.763 |
| LateStg2_021479 | 2880.929 | 2591.085 | 2935.565 | 3095.02 | 2751.518 | 3877.997 | 2929.471 | 2761.362 |
| LateStg2_021485 | 3236.978 | 2840.985 | 3298.95 | 3514.251 | 3336.928 | 4831.199 | 4070.197 | 5153.553 |
| LateStg2_021490 | 2055.398 | 2328.332 | 2304.917 | 2529.798 | 2578.518 | 2827.262 | 2270.409 | 2361.421 |
| LateStg2_021493 | 2089.283 | 1969.834 | 2252.854 | 2541.756 | 2554.018 | 3403.82 | 2142.646 | 2208.38 |
| LateStg2_021498 | 3994.694 | 3345.87 | 3519.226 | 4221.659 | 4350.851 | 4858.934 | 3506.732 | 3779.842 |
| LateStg2_021499 | 2712.5 | 2477.536 | 2535.785 | 3120.787 | 3083.258 | 4619.17 | 3050.456 | 3503.044 |
| LateStg2_021803 | 2568.571 | 2524.476 | 2509.832 | 2820.704 | 2609.458 | 2990.457 | 2279.461 | 2172.796 |
| LateStg2_021817 | 2184.182 | 2720.577 | 2811.529 | 4809.278 | 63866.5 | 4006.276 | 3240.366 | 2969.606 |
| LateStg2_021819 | 2690.661 | 2528.864 | 2868.553 | 3699.345 | 4265.994 | 3856.545 | 3302.492 | 2991.057 |
| LateStg2_021821 | 3862.728 | 5570.37 | 2950.673 | 3903.449 | 4548.635 | 5141.088 | 3771.425 | 3412.051 |
| LateStg2_021828 | 3612.2 | 32668.33 | 13719.52 | 3451.276 | 3465.224 | 5624.391 | 2798.934 | 3131.383 |
| LateStg2_021833 | 988.1922 | 1768.821 | 1152.149 | 1436.283 | 1414.762 | 1567.374 | 1619.189 | 1172.344 |
| LateStg2_021838 | 1659.286 | 2605.402 | 1884.594 | 2379.837 | 2659.232 | 2909.178 | 2613.869 | 2226.825 |
| LateStg2_021841 | 7280.658 | 8393.207 | 6239.35 | 5096.393 | 4927.956 | 7779.298 | 5001.383 | 4618.61 |
| LateStg2_021846 | 1017.125 | 1024.531 | 1290.688 | 1716.599 | 2259.379 | 2301.196 | 1865.429 | 1345.855 |
| LateStg2_021854 | 1631.081 | 2256.517 | 1994.206 | 2089.055 | 2115.195 | 2810.059 | 2315.125 | 1818.905 |
| LateStg2_021856 | 1757.762 | 1863.022 | 1772.417 | 2268.092 | 2477.31 | 2647.954 | 2176.189 | 1961.227 |
| LateStg2_021857 | 3901.552 | 3670.615 | 3626.944 | 4188.205 | 4158.46 | 4751.309 | 3987.096 | 3730.004 |
| LateStg2_021859 | 1470.294 | 1885.057 | 1935.032 | 2271.902 | 2294.143 | 3048.345 | 2117.187 | 1776.545 |
| LateStg2_021860 | 1377.228 | 1912.527 | 1545.966 | 2029.096 | 3843.036 | 2294.62 | 2148.919 | 1831.696 |
| LateStg2_021863 | 1389.546 | 1225.311 | 1220.319 | 1592.922 | 1506.453 | 1743.039 | 37436.57 | 20785.09 |
| LateStg2_021865 | 847.9526 | 72243.9 | 58963.65 | 1584.339 | 1561.829 | 1890.786 | 1446.563 | 1301.552 |
| LateStg2_021868 | 841.1623 | 1730.123 | 1106.992 | 1520.119 | 1326.74 | 1839.049 | 1526.568 | 1216.93 |
| LateStg2_021871 | 4293.53 | 11133.89 | 6076.056 | 6914.307 | 6755.31 | 13075.63 | 5942.146 | 5916.069 |
| LateStg2_021880 | 1790.208 | 1980.132 | 2614.285 | 2057.024 | 2096.017 | 3959.8 | 1832.259 | 1487.634 |
| LateStg2_021881 | 2384.826 | 1999.357 | 2666.056 | 2852.15 | 3033.592 | 3377.481 | 4271.626 | 3901.21 |
| LateStg2_021883 | 2065.41 | 1622.946 | 2569.527 | 2671.253 | 4642.282 | 3220.608 | 2297.504 | 2061.914 |
| LateStg2_021890 | 3022.65 | 1988.88 | 2282.66 | 2251.566 | 2248.979 | 3052.289 | 2415.519 | 2128.133 |
| LateStg2_021891 | 1124.762 | 961.2568 | 1354.482 | 1361.077 | 1241.26 | 1576.201 | 1462.741 | 1096.385 |
| LateStg2_021892 | 2474.483 | 4105.839 | 4501.21 | 2967.051 | 2868.365 | 4078.879 | 2353.423 | 2167.423 |
| LateStg2_021894 | 2221.702 | 1767.34 | 2488.434 | 1923.35 | 2652.345 | 5760.332 | 4985.451 | 1684.064 |
| LateStg2_021897 | 2094.173 | 1872.113 | 2564.54 | 3134.878 | 3177.1 | 3170.251 | 2812.481 | 2291.9 |
| LateStg2_021959 | 1944.575 | 1075.842 | 1370.23 | 6504.857 | 55850.97 | 2370.069 | 1624.237 | 1465.625 |
| LateStg2_021961 | 2126.008 | 2601.519 | 2931.139 | 3868.475 | 3742.739 | 5313.381 | 3308.218 | 3188.797 |
| LateStg2_021499 | 2712.5 | 2477.536 | 2535.785 | 3120.787 | 3083.258 | 4619.17 | 3050.456 | 3503.044 |

| Protein | IGF2BP3 | LRRFIP2 | MAGEA10 | MAGEA4 | MAP2K5 | PTPN20A |
|---|---|---|---|---|---|---|
| Control1_021608 | 1864.379 | 10256.51 | 1707.204 | 1270.237 | 2062.856 | 3315.828 |
| Control1_021611 | 984.5742 | 1642.046 | 949.5056 | 717.2795 | 980.4752 | 1986.406 |
| Control1_021630 | 2495.178 | 3443.966 | 2016.665 | 1715.477 | 2661.723 | 5203.608 |
| Control1_021631 | 1769.07 | 2760.66 | 2086.983 | 2381.255 | 2711.591 | 2212.801 |
| Control1_021642 | 2347.117 | 5726.215 | 2239.27 | 1819.513 | 2590.68 | 5952.694 |
| Control1_021643 | 1577.693 | 2475.029 | 1392.899 | 1095.408 | 1734.76 | 1687.801 |
| Control1_021650 | 1712.624 | 3158.91 | 2166.367 | 3076.028 | 3988.011 | 4640.239 |
| Control1_021660 | 2025.719 | 10491.47 | 1699.251 | 1931.718 | 2483.095 | 2033.607 |
| Control1_021661 | 2947.112 | 4964.015 | 2260.505 | 1715.278 | 2982.163 | 2494.186 |
| Control1_021663 | 2921.823 | 3766.776 | 2208.297 | 1894.731 | 2643.505 | 2964.772 |
| Control1_021674 | 1885.233 | 5666.105 | 1571.638 | 1156.283 | 2244.439 | 2457.223 |
| Control1_021679 | 1483.428 | 2188.354 | 1499.469 | 1066.008 | 2001.052 | 1444.111 |
| Control1_021680 | 2291.881 | 7936.889 | 2166.929 | 1679.373 | 2362.385 | 3149.52 |
| Control1_021681 | 3826.813 | 10582.86 | 6322.936 | 4911.227 | 5544.572 | 13939.81 |
| Control1_021682 | 2371.601 | 4526.223 | 1991.898 | 1569.11 | 4169.351 | 2907.473 |
| Control2_021005 | 6199.772 | 11076.7 | 11112.01 | 9611.882 | 6652.302 | 19237.07 |
| Control2_021007 | 3216.338 | 5692.778 | 3194.603 | 2842.006 | 4446.303 | 3910.378 |
| Control2_021016 | 1894.693 | 3015.911 | 2324.273 | 1948.089 | 2766.241 | 2710.309 |
| Control2_021017 | 1714.025 | 3137.241 | 1811.556 | 1476.747 | 2095.534 | 2572.001 |
| Control2_021025 | 2301.794 | 5997.156 | 1928.071 | 1764.336 | 2214.804 | 2567.013 |
| Control2_021037 | 1786.695 | 11870.33 | 1835.236 | 1526.275 | 2408.522 | 3380.793 |
| Control2_021038 | 1468.671 | 2967.612 | 1523.531 | 1207.042 | 2009.926 | 1666.915 |
| Control2_021045 | 1833.289 | 2309.808 | 2015.164 | 1736.922 | 2395.172 | 2511.068 |
| Control2_021046 | 3026.465 | 6848.673 | 2894.598 | 2433.767 | 3258.239 | 5672.938 |
| Control2_021401 | 2153.839 | 3751.081 | 2099.173 | 2967.183 | 2414.103 | 2164.446 |
| Control2_021405 | 1577.527 | 2708.492 | 1709.801 | 1549.398 | 2091.493 | 2295.796 |
| Control2_021406 | 1377.186 | 3379.66 | 1510.359 | 1138.178 | 1824.205 | 1522.514 |
| Control2_021419 | 1327.409 | 2950.186 | 1288.873 | 1206.732 | 1920.516 | 3093.032 |
| Control2_021420 | 1427.761 | 3191.296 | 1505.299 | 1258.148 | 1800.616 | 1843.103 |
| Control2_021423 | 3570.623 | 6620.853 | 3579.061 | 2918.362 | 4426.904 | 4727.215 |
| Control2_021426 | 2939.504 | 8472.987 | 2853.974 | 2939.304 | 5489.914 | 2777.067 |
| Control2_021430 | 1712.129 | 3839.469 | 1920.69 | 1343.425 | 2271.696 | 2344.028 |
| Control2_021436 | 1492.531 | 2310.743 | 1607.355 | 1279.09 | 1830.218 | 1714.501 |
| Control2_021451 | 2780.555 | 3572.501 | 2861.352 | 2283.854 | 3366.02 | 3448.814 |
| Control2_021453 | 1955.682 | 6044.063 | 2175.041 | 1564.877 | 2295.219 | 2062.084 |
| Control2_021454 | 1577.288 | 2848.697 | 1455.822 | 1306.672 | 1894.117 | 1759.484 |
| Control2_021455 | 2564.917 | 4994.603 | 2530.726 | 1924.332 | 3277.945 | 2818.599 |
| Control2_021463 | 4073.638 | 10964.05 | 4476.259 | 3557.854 | 4176.659 | 5050.335 |
| Control2_021470 | 1751.386 | 5710.754 | 1632.83 | 1402.099 | 2130.032 | 2535.66 |
| Control2_021477 | 3498.464 | 6047.108 | 2975.548 | 2798.397 | 3279.928 | 6853.659 |
| Control2_021478 | 1975.51 | 4209.962 | 3187.603 | 1924.962 | 2382.097 | 3422.74 |
| Control2_021484 | 2030.785 | 4996.788 | 2260.441 | 1635.343 | 2426.476 | 2468.278 |
| Control2_021494 | 1358.159 | 6289.746 | 1428.486 | 1155.8 | 1760.796 | 3294.846 |
| Control2_021495 | 7607.972 | 10760.02 | 8813.236 | 8154.649 | 4858.083 | 8017.285 |
| Control2_021497 | 2022.71 | 2381.643 | 2071.199 | 1836.147 | 2880.86 | 2694.962 |
| Control2_021801 | 2131.438 | 2938.354 | 1934.592 | 1637.284 | 2040.849 | 2076.451 |
| Control2_021802 | 1582.688 | 1721.553 | 1333.201 | 1159.959 | 2289.404 | 5436.568 |
| Control2_021804 | 2513.429 | 4985.019 | 1938.967 | 1565.209 | 2898.718 | 2946.264 |
| Control2_021805 | 1813.176 | 4710.699 | 1744.263 | 1476.581 | 3112.112 | 2164.224 |
| Control2_021806 | 2214.155 | 3592.359 | 3411.004 | 2731.398 | 4373.876 | 4083.713 |
| Control2_021809 | 1756.475 | 3069.143 | 1770.113 | 1568.689 | 2315.789 | 1870.303 |
| Control2_021810 | 2409.725 | 4595.2 | 2483.45 | 2014.932 | 3161.544 | 2782.631 |
| Control2_021811 | 1230.763 | 1702.497 | 1990.884 | 1592.71 | 2056.904 | 2890.082 |
| Control2_021812 | 3736.236 | 6074.945 | 5099.298 | 3351.37 | 5101.794 | 4984.095 |
| Control2_021818 | 1723.422 | 3067.722 | 1692.577 | 2609.891 | 2401.301 | 1806.957 |
| Control2_021822 | 2258.871 | 5106.237 | 2454.382 | 1692.475 | 2726.053 | 2685.046 |
| Control2_021823 | 1782.433 | 5978.676 | 1373.312 | 1059.723 | 2141.575 | 2758.369 |
| Control2_021824 | 1929.38 | 3387.352 | 5770.001 | 3186.82 | 2379.998 | 6112.298 |
| Control2_021825 | 2894.506 | 12133.5 | 3019.027 | 2065.98 | 3470.955 | 3126.288 |
| Control2_021826 | 2975.528 | 3619.189 | 2449.931 | 1765.577 | 2665.075 | 4058.315 |
| Control2_021829 | 1078.297 | 2541.436 | 1017.431 | 829.9832 | 1492.023 | 1211.206 |
| Control2_021831 | 1388.789 | 17426.87 | 1383.007 | 1163.831 | 1803.056 | 2659.547 |
| Control2_021834 | 2731.65 | 3946.079 | 2371.834 | 2448.149 | 3712.545 | 3132.718 |
| Control2_021835 | 1425.636 | 4211.808 | 2462.035 | 1860.317 | 2387.911 | 3136.524 |
| Control2_021836 | 2048.427 | 3871.687 | 1729.768 | 1484.16 | 2294.109 | 1869.645 |
| Control2_021837 | 1745.759 | 2669.79 | 1544.561 | 1699.246 | 2605.019 | 2069.25 |
| Control2_021839 | 2341.041 | 3813.47 | 2127.806 | 1811.276 | 2915.472 | 2172.454 |
| Control2_021840 | 1466.736 | 2785.596 | 1425.364 | 1161.345 | 1872.754 | 1764.8 |
| Control2_021844 | 2580.429 | 5380.09 | 2196.979 | 1899.78 | 3372.265 | 2346.553 |
| Control2_021845 | 2724.959 | 4277.382 | 2346.401 | 1932.341 | 3312.14 | 2775.452 |
| Control2_021848 | 1456.744 | 2689.313 | 1499.23 | 1191.049 | 2159.648 | 6106.434 |
| Control2_021849 | 1987.449 | 3206.646 | 2118.117 | 2061.679 | 4011.126 | 3273.238 |
| Control2_021850 | 2179.94 | 2594.357 | 2051.556 | 1817.563 | 2913.705 | 2595.519 |
| Control2_021851 | 3358.091 | 5593.778 | 3204.729 | 3319.564 | 7174.081 | 5437.286 |
| Control2_021852 | 1963.275 | 2311.181 | 1711.839 | 1746.169 | 2653.722 | 2219.421 |
| Control2_021853 | 1542.191 | 2405.584 | 1254.529 | 2225.92 | 1840.036 | 1395.407 |
| Control2_021855 | 2092.828 | 5193.978 | 1821.72 | 1955.116 | 3112.859 | 2274.963 |
| Control2_021861 | 1766.345 | 2249.844 | 1485.959 | 1441.116 | 2357.153 | 1741.011 |
| Control2_021862 | 1758.152 | 3119.105 | 1388.473 | 1208.254 | 2264.852 | 1470.605 |
| Control2_021864 | 1228.498 | 1617.374 | 1600.64 | 1097.203 | 1773.925 | 1405.638 |
| Control2_021866 | 3340.706 | 3692.312 | 3083.481 | 2305.219 | 4003.694 | 3172.705 |
| Control2_021869 | 2041.283 | 5513.359 | 4128.772 | 3249.421 | 2908.544 | 5007.78 |
| Control2_021870 | 2274.004 | 5381.599 | 3390.585 | 1763.338 | 3202.194 | 2078.389 |
| Control2_021872 | 2353.007 | 7394.805 | 1774.071 | 1518.602 | 2707.483 | 2249.85 |
| Control2_021874 | 2754.599 | 5223.595 | 2337.521 | 1887.844 | 3652.429 | 2367.014 |
| Control2_021875 | 2835.615 | 6109.194 | 1990.811 | 1606.504 | 2701.638 | 2239.367 |
| Control2_021876 | 2026.153 | 5614.535 | 1726.306 | 1692.19 | 2859.931 | 2391.009 |
| Control2_021877 | 2683.601 | 4044.607 | 2816.4 | 2392.499 | 3991.897 | 3151.645 |
| Control2_021882 | 2432.832 | 5635.096 | 2318.964 | 3012.98 | 3779.524 | 3017.525 |
| Control2_021884 | 2162.308 | 4175.698 | 1896.357 | 1930.513 | 2802.656 | 2045.35 |
| Control2_021885 | 2322.551 | 2360.523 | 2020.608 | 2019.363 | 2912.89 | 3519.932 |
| Control2_021887 | 1907.668 | 8062.939 | 1698.869 | 1543.445 | 2905.972 | 2107.129 |
| Control2_021888 | 2362.468 | 6705.703 | 2263.509 | 2009.607 | 3116.232 | 2917.681 |
| Control2_021889 | 1724.205 | 3193.269 | 1365.617 | 1264.229 | 2102.197 | 4013.068 |
| Control2_021893 | 1266.354 | 1532.566 | 1124.324 | 1133.104 | 1673.636 | 1486.05 |
| Control2_021896 | 1786.878 | 3973.447 | 1376.932 | 1401.714 | 2109.678 | 1795.883 |
| Control2_021898 | 2163.932 | 4514.717 | 2123.111 | 1578.816 | 2815.736 | 2178.103 |
| Control2_021899 | 1791.442 | 2999.335 | 1525.146 | 1254.638 | 2230.895 | 2104.384 |
| Control2_021900 | 3060.013 | 3837.566 | 3456.82 | 2574.759 | 3255.242 | 3745.962 |
| Control2_021962 | 2115.775 | 5116.496 | 1706.608 | 1628.084 | 2814.555 | 2084.987 |
| EarlyStg1_021633 | 1599.96 | 3890.38 | 1465.2 | 1037.369 | 1668.078 | 1451.583 |
| EarlyStg1_021651 | 2351.123 | 6750.972 | 2161.845 | 1771.912 | 2854.07 | 2561.211 |
| EarlyStg1_021654 | 2713.403 | 3945.555 | 2002.649 | 1859.56 | 2533.283 | 3189.184 |
| EarlyStg1_021655 | 3210.863 | 6902.838 | 2143.019 | 1764.912 | 3295.438 | 3250.834 |
| EarlyStg1_021662 | 1536.208 | 2295.528 | 1315.652 | 1296.914 | 2313.41 | 1630.089 |
| EarlyStg1_021675 | 3272.398 | 5877.409 | 2722.891 | 1721.796 | 3145.656 | 3171.789 |
| EarlyStg1_021678 | 1243.303 | 5240.118 | 1195.363 | 849.612 | 4169.675 | 1276.357 |
| EarlyStg2_021024 | 1784.729 | 2599.311 | 1926.939 | 1678.941 | 2398.034 | 2452.743 |
| EarlyStg2_021403 | 1193.16 | 2495.09 | 1988.684 | 8670.834 | 1149.056 | 1061.351 |
| EarlyStg2_021435 | 1020.547 | 1300.412 | 1315.539 | 812.8782 | 1992.811 | 12849.7 |
| EarlyStg2_021440 | 1495.375 | 6394.945 | 1536.416 | 1220.193 | 1741.883 | 1759.137 |
| EarlyStg2_021443 | 2716.052 | 3346.902 | 2821.284 | 2199.833 | 4253.328 | 3857.54 |
| EarlyStg2_021458 | 2492.874 | 5134.364 | 2902.898 | 2525.073 | 3200.766 | 7027.378 |
| EarlyStg2_021462 | 1447.512 | 1962.651 | 1353.171 | 1190.796 | 1692.075 | 2083.382 |
| EarlyStg2_021466 | 3539.669 | 3898.782 | 3162.375 | 2048.981 | 7417.409 | 5946.867 |
| EarlyStg2_021489 | 1833.571 | 2224.745 | 1882.389 | 1361.955 | 1828.498 | 2600.137 |
| EarlyStg2_021496 | 2532.979 | 8436.43 | 2620.774 | 2268.858 | 4002.257 | 3331.943 |
| EarlyStg2_021814 | 1184.509 | 2266.998 | 1189.78 | 1175.504 | 1929.441 | 1631.434 |
| EarlyStg2_021815 | 14434.92 | 7528.605 | 5889.771 | 5140.262 | 11153.38 | 23529.81 |
| EarlyStg2_021820 | 1791.473 | 2298.301 | 1780.983 | 1591.671 | 2375.364 | 2462.813 |
| EarlyStg2_021827 | 862.3957 | 1000.234 | 745.288 | 700.6462 | 1164.85 | 862.7676 |
| EarlyStg2_021830 | 1509.441 | 2078.313 | 1378.075 | 1235.311 | 1996.329 | 1743.144 |
| EarlyStg2_021832 | 940.8926 | 955.0221 | 749.3359 | 680.3148 | 1144.174 | 877.5483 |
| EarlyStg2_021842 | 787.061 | 1720.34 | 740.0612 | 1044.132 | 1072.525 | 843.0966 |
| EarlyStg2_021843 | 1227.167 | 1199.951 | 1252.088 | 932.2355 | 1420.142 | 1299.8 |
| EarlyStg2_021847 | 988.6661 | 1455.479 | 1061.089 | 976.3165 | 2364.797 | 1223.133 |
| EarlyStg2_021858 | 3654.264 | 5782.754 | 6309.748 | 4035.687 | 5097.5 | 4892.059 |
| EarlyStg2_021867 | 1471.776 | 2148.901 | 1221.715 | 1187.602 | 1867.458 | 2958.356 |
| EarlyStg2_021873 | 2437.692 | 3159.13 | 6431.019 | 2019.762 | 3573.626 | 2866.673 |
| EarlyStg2_021895 | 1657.107 | 3322.74 | 1374.092 | 1347.937 | 2303.925 | 2011.179 |
| EarlyStg2_021960 | 4473.527 | 6067.125 | 2660.867 | 3715.26 | 5565.572 | 4084.967 |
| LateStg1_021607 | 2639.71 | 4167.536 | 21571.06 | 20747.58 | 2873.485 | 7195.459 |
| LateStg1_021612 | 1283.638 | 1777.645 | 1451.691 | 1189.345 | 1739.08 | 1653.534 |
| LateStg1_021632 | 1799.823 | 3101.039 | 2060.273 | 1602.773 | 5195.655 | 1645.745 |
| LateStg1_021683 | 1143.095 | 2445.638 | 1851.534 | 4298.669 | 8690.543 | 1780.728 |
| LateStg1_021691 | 2924.456 | 8177.706 | 4079.96 | 2844.118 | 2816.506 | 6253.56 |
| LateStg1_021692 | 8336.307 | 2067.293 | 2052.88 | 3534.455 | 2314.055 | 2435.415 |
| LateStg1_021696 | 5254.942 | 7300.942 | 5486.125 | 3686.07 | 10444.32 | 10842.8 |
| LateStg1_021699 | 1463.845 | 1553.582 | 1393.758 | 1065.26 | 1777.222 | 1628.989 |
| LateStg2_021004 | 3447.883 | 6800.578 | 3186.653 | 2627.675 | 3490.451 | 4227.487 |
| LateStg2_021006 | 3785.679 | 12021 | 3061.898 | 3122.281 | 3726.921 | 3191.783 |
| LateStg2_021028 | 4012.385 | 4351.325 | 3588.626 | 3046.919 | 4898.403 | 3893.103 |
| LateStg2_021029 | 4817.28 | 7556.265 | 4689.437 | 4079.114 | 5566.21 | 8339.843 |
| LateStg2_021039 | 4220.891 | 10757.38 | 3931.166 | 3348.218 | 5050.079 | 7727.377 |
| LateStg2_021040 | 2472.303 | 12349.52 | 2486.638 | 2116.17 | 3101.401 | 3078.854 |
| LateStg2_021402 | 1665.538 | 10941.54 | 2164.561 | 3789.045 | 2091.446 | 23209.96 |
| LateStg2_021404 | 2103.374 | 10730.8 | 2301.383 | 1848.497 | 2619.036 | 2114.464 |
| LateStg2_021409 | 2287.658 | 2560.275 | 2146.635 | 1698.201 | 2258.806 | 2988.973 |
| LateStg2_021411 | 3275.688 | 5937.628 | 3641.948 | 2783.715 | 3631.025 | 3842.071 |
| LateStg2_021412 | 1421.224 | 1966.427 | 1540.838 | 1344.967 | 1777.86 | 1717.123 |
| LateStg2_021413 | 1909.862 | 8649.381 | 1902.606 | 1753.411 | 2701.412 | 2172.419 |
| LateStg2_021418 | 1400.382 | 1756.549 | 6483.009 | 12480.7 | 1712.24 | 1907.998 |
| LateStg2_021421 | 2016.597 | 3387.465 | 1986.933 | 1592.037 | 2362.854 | 2431.391 |
| LateStg2_021422 | 2534.09 | 9135.84 | 3163.03 | 2608.115 | 3364.623 | 3781.505 |
| LateStg2_021424 | 2096.701 | 5080.653 | 2221.863 | 1669.388 | 2880.509 | 2746.375 |
| LateStg2_021425 | 3821.13 | 5625.683 | 5187.55 | 33888.13 | 10796.37 | 22975.41 |
| LateStg2_021427 | 2006.091 | 4911.945 | 5642.549 | 3571.163 | 2693.277 | 6688.38 |
| LateStg2_021428 | 1391.924 | 1660.351 | 1548.41 | 1357.469 | 9092.653 | 1680.029 |
| LateStg2_021429 | 2844.512 | 4992.574 | 2967.538 | 2066.671 | 3387.576 | 2708.596 |
| LateStg2_021431 | 2317.118 | 4144.749 | 2499.042 | 2225.565 | 3046.623 | 2595.132 |
| LateStg2_021432 | 2551.755 | 4449.926 | 2553.865 | 1790.208 | 2791.503 | 2231.627 |
| LateStg2_021433 | 3632.759 | 7242.118 | 3571.259 | 2868.022 | 4197.165 | 4657.339 |
| LateStg2_021437 | 3358.309 | 36422.07 | 3309.372 | 2486.033 | 4058.445 | 5346.232 |
| LateStg2_021441 | 3184.235 | 23989.57 | 3092.49 | 2360.327 | 4081.13 | 3894.148 |
| LateStg2_021442 | 1953.693 | 3097.947 | 2175.145 | 1574.884 | 2440.885 | 1844.334 |
| LateStg2_021450 | 62465.09 | 9856.237 | 6436.29 | 5774.264 | 8325.764 | 7744.436 |
| LateStg2_021452 | 2628.204 | 7171.939 | 2770.725 | 2191.751 | 3443.649 | 2911.636 |
| LateStg2_021456 | 2295.275 | 2391.687 | 1978.448 | 1735.68 | 3055.247 | 2944.797 |
| LateStg2_021457 | 6740.558 | 13294.24 | 7297.811 | 7370.232 | 26310.33 | 10478.36 |
| LateStg2_021459 | 1799.142 | 3100.266 | 1814.398 | 1634.251 | 2973.406 | 2348.097 |
| LateStg2_021467 | 3031.402 | 9162.167 | 3030.005 | 3013.527 | 5899.702 | 3568.804 |
| LateStg2_021468 | 2555.232 | 5898.635 | 2403.385 | 2061.466 | 3099.336 | 3734.919 |
| LateStg2_021469 | 1346.583 | 2408.616 | 1284.269 | 1075.315 | 1683.193 | 1550.628 |
| LateStg2_021471 | 2345.696 | 6284.123 | 2197.31 | 3165.057 | 2970.156 | 2605.741 |
| LateStg2_021476 | 13932.98 | 5353.104 | 4163.6 | 2583.433 | 3163.012 | 14982.35 |
| LateStg2_021479 | 2786.301 | 3761.414 | 3220.481 | 2663.487 | 3713.563 | 16733.18 |
| LateStg2_021485 | 3572.308 | 22588.2 | 3824.449 | 3019.581 | 4860.078 | 5384.661 |
| LateStg2_021490 | 2700.272 | 5644.643 | 2608.352 | 2090.352 | 3147.381 | 3369.221 |
| LateStg2_021493 | 2513.628 | 4698.848 | 2510.315 | 2021.454 | 2975.269 | 2943.559 |
| LateStg2_021498 | 4285.45 | 7355.246 | 4261.771 | 3229.588 | 5198.682 | 4788.694 |
| LateStg2_021499 | 3477.192 | 5287.922 | 3132.654 | 2428.364 | 3670.777 | 4016.163 |
| LateStg2_021803 | 2550.41 | 9028.317 | 2471.884 | 2004.119 | 3726.039 | 3126.381 |
| LateStg2_021817 | 3489.376 | 7562.101 | 2882.902 | 2628.133 | 4314.625 | 3248.255 |
| LateStg2_021819 | 3685.966 | 6583.73 | 3108.269 | 2545.667 | 4423.612 | 3748.161 |
| LateStg2_021821 | 3855.53 | 7804.9 | 3294.47 | 2903.67 | 4701.069 | 3552.171 |
| LateStg2_021828 | 3411.682 | 7526.149 | 3897.674 | 3308.141 | 5139.618 | 4285.685 |
| LateStg2_021833 | 1279.158 | 2007.471 | 1216.094 | 974.8655 | 1689.938 | 1283.639 |
| LateStg2_021838 | 2430.028 | 2848.576 | 2289.099 | 1881.045 | 2790.638 | 2913.932 |
| LateStg2_021841 | 4848.952 | 7746.386 | 7852.988 | 5259.368 | 6938.347 | 7712.883 |
| LateStg2_021846 | 2159.849 | 3125.473 | 1474.7 | 1227.702 | 2012.191 | 1497.391 |
| LateStg2_021854 | 2089.664 | 2749.929 | 2461.663 | 2074.198 | 2346.469 | 2996.54 |
| LateStg2_021856 | 2287.684 | 4127.567 | 2147.37 | 1690.916 | 2545.96 | 2611.793 |
| LateStg2_021857 | 3967.139 | 5754.583 | 3596.755 | 3645.516 | 5079.897 | 5358.171 |
| LateStg2_021859 | 2313.868 | 3634.111 | 1929.03 | 1781.592 | 2948.952 | 2406.479 |
| LateStg2_021860 | 1964.879 | 2275.137 | 1656.999 | 1510.77 | 2197.393 | 2036.264 |
| LateStg2_021863 | 1770.846 | 2499.974 | 1226.013 | 1140.628 | 1928.769 | 1697.19 |
| LateStg2_021865 | 1523.649 | 2533.475 | 1172.621 | 1082.984 | 2098.228 | 1465.648 |
| LateStg2_021868 | 1359.645 | 2195.805 | 1229.105 | 1027.971 | 1746.367 | 1267.317 |
| LateStg2_021871 | 7184.004 | 15378.62 | 6834.217 | 5741.78 | 10556.95 | 7021.684 |
| LateStg2_021880 | 2133.393 | 4583.188 | 1977.947 | 1747.046 | 2667.728 | 2526.626 |
| LateStg2_021881 | 2895.45 | 7062.843 | 2719.433 | 2410.467 | 3115.291 | 4520.945 |
| LateStg2_021883 | 2484.5 | 16564.51 | 2198.001 | 2264.344 | 3763.834 | 17526.91 |
| LateStg2_021890 | 2178.214 | 16641.9 | 2523.614 | 2173.581 | 3724.101 | 2897.678 |
| LateStg2_021891 | 1186.718 | 1491.571 | 1603.177 | 1328.941 | 3133.506 | 3413.422 |
| LateStg2_021892 | 2937.444 | 5562.712 | 3328.625 | 2585.967 | 4427.688 | 3443.103 |
| LateStg2_021894 | 1890.908 | 3334.574 | 2396.437 | 2163.834 | 2887.473 | 3018.096 |
| LateStg2_021897 | 3112.535 | 6338.442 | 2545.71 | 2285.715 | 3852.078 | 3186.883 |
| LateStg2_021959 | 1477.145 | 6609.375 | 1137.616 | 1249.313 | 4849.732 | 1808.159 |
| LateStg2_021961 | 3932.856 | 8437.228 | 3262.102 | 2669.569 | 4774.873 | 3491.703 |
| LateStg2_021499 | 3477.192 | 5287.922 | 3132.654 | 2428.364 | 3670.777 | 4016.163 |

| Protein | RAD23B | STAT1 | TPM1 | XAGE1D | ZNRD1 |
|---|---|---|---|---|---|
| Control1_021608 | 2081.126 | 2617.833 | 4049.264 | 1505.695 | 2626.203 |
| Control1_021611 | 905.2051 | 977.5851 | 1796.781 | 863.7998 | 1532.123 |
| Control1_021630 | 1990.034 | 3163.671 | 6107.433 | 1897.377 | 3821.562 |
| Control1_021631 | 2347.861 | 2764.548 | 4874.361 | 1577.602 | 2855.739 |
| Control1_021642 | 2026.351 | 6089.905 | 4518.586 | 1985.198 | 4152.455 |
| Control1_021643 | 1234.18 | 2229.621 | 2248.575 | 1155.519 | 2530.437 |
| Control1_021650 | 2266.734 | 9483.072 | 4720.325 | 2449.356 | 5897.559 |
| Control1_021660 | 1338.988 | 3689.077 | 4882.12 | 1281.009 | 3234.591 |
| Control1_021661 | 2421.713 | 3949.958 | 6032.581 | 1948.014 | 3304.639 |
| Control1_021663 | 4486.685 | 3322.445 | 3789.802 | 2135.498 | 4907.796 |
| Control1_021674 | 1833.585 | 2449.947 | 8639.902 | 1360.602 | 4240.082 |
| Control1_021679 | 1802.526 | 1950.754 | 2527.683 | 1278.041 | 2860.415 |
| Control1_021680 | 2543.393 | 15907.4 | 4196.795 | 1774.904 | 3386.469 |
| Control1_021681 | 4156.378 | 9128.958 | 19809.27 | 6150.266 | 9369.373 |
| Control1_021682 | 2309.431 | 2437.564 | 3893.909 | 1728.037 | 3301.781 |
| Control2_021005 | 5670.086 | 4960.157 | 22784.55 | 18345.03 | 20955.56 |
| Control2_021007 | 17546.81 | 7313.667 | 7748.537 | 3598.932 | 4526.242 |
| Control2_021016 | 2141.714 | 3366.335 | 5555.07 | 2589.406 | 3272.007 |
| Control2_021017 | 1747.83 | 3847.813 | 4092.125 | 2194.708 | 2639.944 |
| Control2_021025 | 1995.162 | 3132.801 | 4412.822 | 2386.34 | 2807.273 |
| Control2_021037 | 1679.377 | 3354.587 | 4200.855 | 2184.704 | 3093.381 |
| Control2_021038 | 1384.307 | 2710.608 | 3518.954 | 1880.842 | 2744.047 |
| Control2_021045 | 1973.987 | 1904.59 | 3795.172 | 2411.016 | 2970.695 |
| Control2_021046 | 2410.957 | 3980.489 | 5714.518 | 4737.667 | 3939.826 |
| Control2_021401 | 5811.137 | 3107.932 | 7447.278 | 2357.483 | 3127.564 |
| Control2_021405 | 1536.453 | 2455.628 | 3139.008 | 1753.436 | 2103.498 |
| Control2_021406 | 2339.278 | 3625.717 | 3868.642 | 1370.279 | 1894.139 |
| Control2_021419 | 1303.955 | 3515.891 | 2877.05 | 1374.645 | 1776.012 |
| Control2_021420 | 1192.939 | 3347.262 | 3224.197 | 1403.629 | 1900.957 |
| Control2_021423 | 3432.053 | 4868.726 | 11813.17 | 4171.47 | 5555.068 |
| Control2_021426 | 3783.557 | 6349.507 | 6183.704 | 3560.529 | 7570.511 |
| Control2_021430 | 1525.482 | 2578.638 | 70438.51 | 1758.205 | 2714.357 |
| Control2_021436 | 1358.224 | 2193.62 | 2708.096 | 1785.438 | 2455.876 |
| Control2_021451 | 3060.993 | 4270.824 | 6303.973 | 2949.225 | 8755.411 |
| Control2_021453 | 2084.262 | 3021.721 | 6835.281 | 1951.169 | 3240.717 |
| Control2_021454 | 2367.539 | 2441.304 | 4791.178 | 1549.041 | 2289.409 |
| Control2_021455 | 2358.646 | 4807.185 | 6212.127 | 2661.667 | 3698.437 |
| Control2_021463 | 3590.858 | 7434.23 | 9568.385 | 4946.078 | 5981.495 |
| Control2_021470 | 1462.485 | 2516.297 | 10171.16 | 1667.289 | 2791.111 |
| Control2_021477 | 2609.898 | 4227.737 | 7400.694 | 3379.937 | 3876.3 |
| Control2_021478 | 1919.419 | 6160.237 | 4680.497 | 3213.554 | 3278.552 |
| Control2_021484 | 1856.142 | 3234.766 | 4323.386 | 2336.339 | 2965.36 |
| Control2_021494 | 1606.667 | 3721.188 | 2527.812 | 1406.064 | 2014.624 |
| Control2_021495 | 10114.45 | 9327.442 | 13563.37 | 10985.96 | 11552.79 |
| Control2_021497 | 8194.55 | 4910.062 | 4861.554 | 2291.933 | 3220.736 |
| Control2_021801 | 2481.315 | 2854.822 | 3918.524 | 2763.663 | 2642.432 |
| Control2_021802 | 1385.581 | 3183.812 | 2829.928 | 1533.262 | 3402.505 |
| Control2_021804 | 2550.711 | 2801.768 | 4235.614 | 2523.631 | 5712.794 |
| Control2_021805 | 2154.696 | 3056.303 | 4473.074 | 1850.957 | 5102.744 |
| Control2_021806 | 2606.994 | 5057.475 | 5208.636 | 2913.279 | 7700.55 |
| Control2_021809 | 3831.109 | 2834.949 | 3286.348 | 1695.697 | 2539.116 |
| Control2_021810 | 2098.649 | 3753.405 | 4439.834 | 2398.99 | 3046.975 |
| Control2_021811 | 1837.785 | 2695.945 | 3664.561 | 1835.566 | 1736.29 |
| Control2_021812 | 3646.176 | 6592.742 | 7476.003 | 4860.687 | 5299.261 |
| Control2_021818 | 1766.938 | 13912.53 | 2448.699 | 1935.279 | 5124.145 |
| Control2_021822 | 1965.292 | 3332.935 | 3820.505 | 2458.486 | 4966.497 |
| Control2_021823 | 1343.488 | 2322.024 | 2924.873 | 1400.678 | 3526.075 |
| Control2_021824 | 1756.883 | 6932.623 | 12069.42 | 5871.825 | 7241.109 |
| Control2_021825 | 2659.892 | 3660.737 | 4434.643 | 3017.324 | 6072.551 |
| Control2_021826 | 1588.61 | 3918.005 | 5187.333 | 2401.518 | 3481.845 |
| Control2_021829 | 974.2268 | 1802.962 | 2828.033 | 1128.866 | 2941.595 |
| Control2_021831 | 1234.548 | 2592.773 | 3259.55 | 1438.736 | 2054.688 |
| Control2_021834 | 2709.399 | 4566.653 | 5304.744 | 2605.529 | 7395.801 |
| Control2_021835 | 1843.991 | 4033.994 | 3249.438 | 2702.522 | 3695.011 |
| Control2_021836 | 2803.469 | 3491.743 | 3627.928 | 1791.521 | 2439.615 |
| Control2_021837 | 1769.233 | 3720.748 | 3504.717 | 1782.507 | 4207.138 |
| Control2_021839 | 2231.778 | 3304.446 | 3685.084 | 2180.862 | 5415.791 |
| Control2_021840 | 1310.607 | 2112.964 | 2436.706 | 1458.616 | 3747.051 |
| Control2_021844 | 2283.847 | 3591.101 | 5118.613 | 2345.115 | 5396.899 |
| Control2_021845 | 2272.65 | 4054.93 | 6627.734 | 2873.661 | 6528.291 |
| Control2_021848 | 1689.162 | 11687.5 | 2823.277 | 1698.773 | 4285.151 |
| Control2_021849 | 3212.107 | 4313.128 | 4844.187 | 1940.267 | 5146.62 |
| Control2_021850 | 1710.077 | 2985.272 | 10907.08 | 2022.729 | 4563.897 |
| Control2_021851 | 2906.247 | 5124.673 | 6914.633 | 3478.561 | 5951.68 |
| Control2_021852 | 1947.486 | 3140.212 | 3194.615 | 2367.34 | 5520.482 |
| Control2_021853 | 1300.163 | 3414.311 | 2726.988 | 1324.848 | 4586.414 |
| Control2_021855 | 4717.033 | 3128.178 | 4070.028 | 2258.982 | 6357.409 |
| Control2_021861 | 1846.351 | 3192.134 | 3243.591 | 1926.237 | 3090.701 |
| Control2_021862 | 1345.939 | 2384.382 | 2742.069 | 1728.213 | 3670.252 |
| Control2_021864 | 1423.723 | 2487.983 | 2064.618 | 1366.074 | 2795.789 |
| Control2_021866 | 3376.394 | 5048.73 | 4946.702 | 3457.549 | 6293.909 |
| Control2_021869 | 2079.015 | 5426.798 | 5380.724 | 4940.874 | 4144.626 |
| Control2_021870 | 2362.266 | 4841.695 | 4649.188 | 2360.201 | 5403.713 |
| Control2_021872 | 1797.54 | 3300.761 | 4053.954 | 2168.691 | 4660.637 |
| Control2_021874 | 2208.745 | 3585.306 | 5739.748 | 2704.132 | 5517.491 |
| Control2_021875 | 1994.916 | 3145.048 | 3000.913 | 2236.848 | 5457.432 |
| Control2_021876 | 3490.562 | 2938.155 | 4877.939 | 1748.16 | 2883.442 |
| Control2_021877 | 2617.009 | 6545.128 | 13734.66 | 3055.266 | 4792.461 |
| Control2_021882 | 2502.86 | 9005.231 | 6358.341 | 2580.803 | 4540.223 |
| Control2_021884 | 2109.724 | 3084.8 | 5077.241 | 2120.757 | 3006.417 |
| Control2_021885 | 3397.924 | 3343.496 | 4043.697 | 2422.662 | 3089.727 |
| Control2_021887 | 1760.354 | 2829.737 | 6973.591 | 1813.83 | 2448.871 |
| Control2_021888 | 2180.773 | 10092.09 | 6037.169 | 2303.328 | 3242.51 |
| Control2_021889 | 1402.45 | 2661.556 | 4983.02 | 1468.28 | 2082.255 |
| Control2_021893 | 1290.029 | 2445.214 | 2708.811 | 1151.813 | 2311.744 |
| Control2_021896 | 1920 | 2577.86 | 5293.315 | 1487.054 | 2189.525 |
| Control2_021898 | 1781.437 | 15068.83 | 4513.11 | 1985.627 | 2620.897 |
| Control2_021899 | 1492.37 | 2394.735 | 3072.453 | 1643.78 | 2270.203 |
| Control2_021900 | 2716.952 | 4014.755 | 5178.692 | 2951.938 | 7874.77 |
| Control2_021962 | 2067.581 | 5010.089 | 4188.351 | 1975.819 | 3179.895 |
| EarlyStg1_021633 | 10190.59 | 2556.25 | 3108.472 | 22871.57 | 2088.815 |
| EarlyStg1_021651 | 2358.632 | 2967.15 | 3584.864 | 1801.215 | 6011.781 |
| EarlyStg1_021654 | 2115.876 | 2382.979 | 3204.194 | 1758.753 | 5859.363 |
| EarlyStg1_021655 | 2346.357 | 2888.672 | 5218.852 | 2056.624 | 4117.299 |
| EarlyStg1_021662 | 1478.589 | 7011.551 | 2377.041 | 18653.11 | 1784.796 |
| EarlyStg1_021675 | 2286.916 | 3358.434 | 5073.502 | 2378.691 | 6228.465 |
| EarlyStg1_021678 | 1124.136 | 1342.022 | 2590.081 | 2450.418 | 2513.441 |
| EarlyStg2_021024 | 1825.564 | 3177.2 | 3875.282 | 2259.64 | 2789.643 |
| EarlyStg2_021403 | 826.697 | 1508.257 | 3280.338 | 1056.332 | 1462.285 |
| EarlyStg2_021435 | 3039.989 | 1482.107 | 1708.522 | 1111.212 | 2970.026 |
| EarlyStg2_021440 | 1274.902 | 2116.541 | 3047.862 | 1489.374 | 2207.21 |
| EarlyStg2_021443 | 6291.922 | 3782.145 | 5357.531 | 2910.532 | 4271.11 |
| EarlyStg2_021458 | 2891.019 | 3304.315 | 5084.472 | 3052.856 | 3851.58 |
| EarlyStg2_021462 | 1220.256 | 1848.675 | 3257.003 | 1419.641 | 1844.949 |
| EarlyStg2_021466 | 2380.558 | 3931.357 | 4237.54 | 5538.389 | 4529.409 |
| EarlyStg2_021489 | 1708.257 | 2492.632 | 3881.379 | 2120.155 | 2455.012 |
| EarlyStg2_021496 | 3119.467 | 4494.802 | 6993.668 | 2825.078 | 3852.443 |
| EarlyStg2_021814 | 1240.605 | 2024.513 | 2875.169 | 1276.932 | 1868.431 |
| EarlyStg2_021815 | 4652.537 | 8918.846 | 13542.18 | 10916.19 | 29827.34 |
| EarlyStg2_021820 | 1846.98 | 2687.747 | 3147.172 | 1999.272 | 4358.43 |
| EarlyStg2_021827 | 735.5688 | 1349.104 | 1553.147 | 807.8251 | 2005.31 |
| EarlyStg2_021830 | 1373.751 | 2402.751 | 2745.201 | 1520.204 | 1758.649 |
| EarlyStg2_021832 | 693.6878 | 1215.148 | 1877.743 | 821.4908 | 989.1862 |
| EarlyStg2_021842 | 732.0325 | 1241.119 | 1438.012 | 792.5935 | 1880.585 |
| EarlyStg2_021843 | 1218.456 | 1565.295 | 1760.286 | 1234.466 | 2293.205 |
| EarlyStg2_021847 | 916.262 | 1675.788 | 1953.103 | 1106.615 | 2237.163 |
| EarlyStg2_021858 | 3918.399 | 5739.398 | 7182.912 | 4633.46 | 11607.87 |
| EarlyStg2_021867 | 1508.762 | 2039.943 | 2430.543 | 1407.597 | 3551.464 |
| EarlyStg2_021873 | 2360.751 | 3891.723 | 6348.432 | 2662.724 | 6903.598 |
| EarlyStg2_021895 | 1259.669 | 2291.862 | 2859.402 | 1732.711 | 1940.729 |
| EarlyStg2_021960 | 5344.483 | 5542.575 | 7626.605 | 4567.441 | 6935.041 |
| LateStg1_021607 | 2548.429 | 3653.705 | 11731.3 | 2238.342 | 9084.524 |
| LateStg1_021612 | 1226.084 | 4710.854 | 27371.76 | 24821.54 | 2435.91 |
| LateStg1_021632 | 2239.28 | 1861.048 | 3672.684 | 11641.46 | 2496.863 |
| LateStg1_021683 | 1150.62 | 2402.067 | 3062.754 | 1141.802 | 2028.778 |
| LateStg1_021691 | 2437.998 | 7010.066 | 4723.274 | 4053.18 | 6519.304 |
| LateStg1_021692 | 11902.46 | 6559.493 | 12545.8 | 1963.734 | 11183.95 |
| LateStg1_021696 | 3874.061 | 6828.621 | 7978.743 | 5397.418 | 7341.834 |
| LateStg1_021699 | 1197.207 | 12961.49 | 3163.428 | 1263.17 | 2505.567 |
| LateStg2_021004 | 2755.789 | 2133.374 | 7471.008 | 15774.18 | 4833.175 |
| LateStg2_021006 | 3055.728 | 5022.605 | 13003.68 | 3081.717 | 4111.345 |
| LateStg2_021028 | 4476.661 | 3874.278 | 6939.209 | 15318.87 | 4612.729 |
| LateStg2_021029 | 3731.415 | 3904.665 | 18153.11 | 5939.745 | 9798.426 |
| LateStg2_021039 | 3605.788 | 4050.392 | 11286.33 | 31136.4 | 10124.7 |
| LateStg2_021040 | 2011.853 | 2894.685 | 6237.084 | 12377.03 | 3488.47 |
| LateStg2_021402 | 3864.334 | 2131.699 | 16814.28 | 1855.743 | 2460.073 |
| LateStg2_021404 | 2806.652 | 3262.728 | 5923.29 | 2109.56 | 2837.274 |
| LateStg2_021409 | 6123.263 | 45207.66 | 3710.094 | 2240.805 | 2672.31 |
| LateStg2_021411 | 2780.356 | 9740.508 | 8277.123 | 86593.27 | 4647.849 |
| LateStg2_021412 | 1367.526 | 2384.968 | 2527.974 | 4301.716 | 2051.114 |
| LateStg2_021413 | 2608.381 | 3445.016 | 26526.73 | 2274.128 | 2877.102 |
| LateStg2_021418 | 1232.774 | 2193.431 | 2718.157 | 1707.805 | 2045.315 |
| LateStg2_021421 | 1757.695 | 7521.871 | 6986.991 | 2146.607 | 2784.716 |
| LateStg2_021422 | 2337.046 | 6197.789 | 5642.467 | 51156.32 | 3961.097 |
| LateStg2_021424 | 2017.996 | 3606.207 | 4320.564 | 81892.36 | 3396.019 |
| LateStg2_021425 | 17817.6 | 8637.526 | 10970.58 | 4581.09 | 7241.283 |
| LateStg2_021427 | 2318.45 | 6676.587 | 9097.011 | 5138.452 | 3666.584 |
| LateStg2_021428 | 1365.524 | 3474.558 | 2805.974 | 1418.446 | 3555.568 |
| LateStg2_021429 | 2401.982 | 3614.203 | 5568.374 | 3001.216 | 4089.902 |
| LateStg2_021431 | 2159.14 | 4110.286 | 5855.135 | 2399.371 | 4049.696 |
| LateStg2_021432 | 2113.099 | 3429.028 | 4533.118 | 2518.522 | 3669.681 |
| LateStg2_021433 | 3288.039 | 17338.42 | 7431.575 | 3735.118 | 5408.98 |
| LateStg2_021437 | 5720.515 | 51111.89 | 6558.229 | 73076.88 | 5622.161 |
| LateStg2_021441 | 7333.667 | 5608.731 | 12014.06 | 3198.293 | 5385.842 |
| LateStg2_021442 | 3378.815 | 2767.79 | 3709.88 | 1951.919 | 4524.983 |
| LateStg2_021450 | 9800.541 | 10278.63 | 11756.66 | 8106.801 | 13587.36 |
| LateStg2_021452 | 2955.094 | 3955.263 | 26551.05 | 3107.26 | 4242.407 |
| LateStg2_021456 | 7126.781 | 3647.837 | 4024.424 | 2143.845 | 3360.468 |
| LateStg2_021457 | 6932.051 | 9832.866 | 19790.06 | 7344.632 | 13282.43 |
| LateStg2_021459 | 1709.713 | 2914.202 | 3567.059 | 1904.825 | 2874.884 |
| LateStg2_021467 | 2425.005 | 5735.723 | 5177.811 | 3191.262 | 4545.997 |
| LateStg2_021468 | 2250.546 | 3828.926 | 9905.679 | 2752.943 | 3839.993 |
| LateStg2_021469 | 1463.695 | 2068.044 | 2449.744 | 1328.585 | 2466.248 |
| LateStg2_021471 | 2245.675 | 3627.445 | 14696.91 | 2467.378 | 3167.771 |
| LateStg2_021476 | 2189.458 | 4731.425 | 5321.727 | 21050.91 | 5567.529 |
| LateStg2_021479 | 2874.51 | 4326.346 | 7389.014 | 3331.391 | 4580.993 |
| LateStg2_021485 | 7718.838 | 6508.286 | 11009.77 | 3845.124 | 6364.518 |
| LateStg2_021490 | 3426.851 | 3655.384 | 9930.515 | 2640.637 | 3786.199 |
| LateStg2_021493 | 2136.006 | 54080.8 | 4759.46 | 2612.632 | 3790.428 |
| LateStg2_021498 | 3818.572 | 5490.723 | 12841.46 | 5246.952 | 6432.803 |
| LateStg2_021499 | 2945.576 | 8863.933 | 5309.148 | 3196.865 | 4388.622 |
| LateStg2_021803 | 2455.32 | 3660.465 | 5247.491 | 2842.318 | 5724.04 |
| LateStg2_021817 | 5146.921 | 6176.601 | 6944.245 | 3376.811 | 9550.769 |
| LateStg2_021819 | 3673.165 | 5193.176 | 6400.219 | 3449.647 | 7443.841 |
| LateStg2_021821 | 3290.416 | 5084.206 | 5759.796 | 3728.741 | 8677.661 |
| LateStg2_021828 | 3254.309 | 7498.517 | 9501.756 | 4700.525 | 9935.705 |
| LateStg2_021833 | 1469.673 | 2038.617 | 2763.421 | 1149.49 | 1673.634 |
| LateStg2_021838 | 1876.231 | 3387.326 | 4041.425 | 4749.024 | 5877.246 |
| LateStg2_021841 | 4960.22 | 9289.899 | 9842.119 | 12962.39 | 12360.34 |
| LateStg2_021846 | 1289.456 | 2194.223 | 3448.56 | 1486.978 | 3482.839 |
| LateStg2_021854 | 1614.691 | 2547.062 | 15433.09 | 2182.438 | 2600.972 |
| LateStg2_021856 | 1702.953 | 2811.449 | 6186.714 | 2217.469 | 4138.507 |
| LateStg2_021857 | 4070.815 | 12466.72 | 8308.568 | 4216.411 | 8964.893 |
| LateStg2_021859 | 1991.047 | 3284.598 | 3241.526 | 2186.996 | 4791.754 |
| LateStg2_021860 | 1510.892 | 2753.512 | 3284.302 | 4857.857 | 3644.717 |
| LateStg2_021863 | 1274.661 | 2135.424 | 5495.126 | 77817.12 | 3516.442 |
| LateStg2_021865 | 1554.264 | 2245.101 | 2560.516 | 15878.09 | 2716.509 |
| LateStg2_021868 | 1314.046 | 2054.09 | 2606.964 | 1274.319 | 3338.825 |
| LateStg2_021871 | 7260.707 | 32976.48 | 12429.49 | 8715.363 | 19074.28 |
| LateStg2_021880 | 1632.411 | 3510.768 | 4512.184 | 2456.451 | 3226.212 |
| LateStg2_021881 | 2407.219 | 4258.419 | 12393.42 | 2759.778 | 4259.74 |
| LateStg2_021883 | 2545.744 | 4550.17 | 6416.215 | 2650.601 | 3773.043 |
| LateStg2_021890 | 2109.595 | 7377.704 | 5154.293 | 2388.609 | 3284.202 |
| LateStg2_021891 | 1392.966 | 3067.306 | 2590.952 | 1235.433 | 1900.166 |
| LateStg2_021892 | 2566.329 | 4587.612 | 6758.511 | 3181.595 | 4032.283 |
| LateStg2_021894 | 2241.106 | 4094.685 | 4474.155 | 5663.077 | 2795.796 |
| LateStg2_021897 | 2396.901 | 6763.967 | 6534.485 | 2598.865 | 3976.887 |
| LateStg2_021959 | 1777.565 | 2373.879 | 3499.995 | 1359.307 | 2181.833 |
| LateStg2_021961 | 3494.667 | 5256.041 | 6366.071 | 3609.642 | 5268.444 |
| LateStg2_021499 | 2945.576 | 8863.933 | 5309.148 | 3196.865 | 4388.622 |

TABLE 7

| | ROC | Variables | Sens | Spec | ROCSD | SensSD | SpecSD | biomarker.panel | AUC |
|---|---|---|---|---|---|---|---|---|---|
| 1 | 0.832 | 7 | 0.753 | 0.721 | 0.051 | 0.046 | 0.216 | XAGE1D, LRRFIP2, MAGEA10, | 0.818 |
| | | | | | | | | GAGE2C, STAT1, ZNRD1, RAD23B | |
| 2 | 0.819 | 5 | 0.723 | 0.752 | 0.044 | 0.123 | 0.091 | XAGE1D, LRRFIP2, STAT1, FADD, RAD23B | 0.812 |
| 3 | 0.821 | 6 | 0.737 | 0.752 | 0.089 | 0.126 | 0.127 | XAGE1D, LRRFIP2, STAT1, | 0.812 |
| | | | | | | | | GAGE1, FADD, RAD23B | |
| 4 | 0.821 | 11 | 0.770 | 0.623 | 0.081 | 0.126 | 0.059 | XAGE1D, LRRFIP2, GAGE2C, DDX43, | 0.812 |
| | | | | | | | | STAT1, CT47A1, GAGE1, MAP2K5, | |
| | | | | | | | | CTAG2, FADD, RAD23B | |
| 5 | 0.814 | 8 | 0.782 | 0.738 | 0.063 | 0.051 | 0.121 | XAGE1D, LRRFIP2, GAGE1, MAGEA4, | 0.811 |
| | | | | | | | | STAT1, ZNRD1, CTAG2, CTAG1A | |
| 6 | 0.816 | 7 | 0.756 | 0.723 | 0.057 | 0.106 | 0.086 | XAGE1D, DDX43, LRRFIP2, GAGE1, | 0.809 |
| | | | | | | | | GAGE2C, STAT1, PTPN20A | |
| 7 | 0.818 | 8 | 0.754 | 0.681 | 0.083 | 0.119 | 0.107 | XAGE1D, LRRFIP2, DDX43, MAGEA10, | 0.809 |
| | | | | | | | | GAGE2C, STAT1, CTAG2, ZNRD1 | |
| 8 | 0.819 | 6 | 0.740 | 0.799 | 0.117 | 0.138 | 0.091 | XAGE1D, LRRFIP2, MAGEA10, | 0.808 |
| | | | | | | | | STAT1, RAD23B, CTAG2 | |
| 9 | 0.828 | 7 | 0.737 | 0.754 | 0.067 | 0.126 | 0.107 | XAGE1D, CT47A1, LRRFIP2, GAGE1, | 0.808 |
| | | | | | | | | STAT1, ZNRD1, RAD23B | |
| 10 | 0.815 | 11 | 0.768 | 0.753 | 0.031 | 0.117 | 0.085 | XAGE1D, LRRFIP2, GAGE2C, CT47A1, | 0.808 |
| | | | | | | | | STAT1, GAGE1, MAGEA4, | |
| | | | | | | | | ZNRD1, DDX53, MAP2K5, RAD23B | |
| 11 | 0.807 | 8 | 0.695 | 0.738 | 0.058 | 0.141 | 0.044 | XAGE1D, LRRFIP2, DDX43, STAT1, | 0.807 |
| | | | | | | | | GAGE2C, MAGEA10, GAGE1, FADD | |
| 12 | 0.813 | 5 | 0.742 | 0.738 | 0.093 | 0.146 | 0.131 | XAGE1D, LRRFIP2, STAT1, FADD, CTAG2 | 0.807 |
| 13 | 0.809 | 9 | 0.766 | 0.769 | 0.081 | 0.135 | 0.115 | XAGE1D, LRRFIP2, DDX43, | 0.807 |
| | | | | | | | | GAGE1, STAT1, MAGEA4, | |
| | | | | | | | | CTAG2, RAD23B, FADD | |
| 14 | 0.826 | 8 | 0.724 | 0.740 | 0.086 | 0.179 | 0.138 | XAGE1D, LRRFIP2, DDX43, | 0.807 |
| | | | | | | | | GAGE2C, CT47A1, STAT1, | |
| | | | | | | | | CTAG2, RAD23B | |

What is claimed is:

1-19. (canceled)

20. A method for diagnosing Non-Small Cell Lung Cancer from a sample comprising serum and/or plasma extracted from a subject, comprising the steps of:
(i) testing the sample for the presence of biomarkers specific for Non-Small Cell Lung Cancer;
(ii) determining whether the subject has Non-Small Cell Lung Cancer based on the detection of said biomarkers;
characterised in that the biomarkers are autoantibodies to antigens comprising XAGE1D, LRRFIP2 and GAGE2C.

21. The method according to claim 20 wherein the antigens further comprise one or more of DDX53, DDX43, GAGE1, MAGEA10, ZNRD1, MAP2K5, MAGEA4, STAT1, CT47A1, IGF2BP3, CTAG2, RAD23B, FADD, PTPN20A, TPM1, CTAG1A.

22. The method according to claim 20 wherein the antigens are biotinylated proteins.

23. The method according to claim 22 wherein each biotinylated protein is formed from a Biotin Carboxyl Carrier Protein folding marker which is fused in-frame with a protein.

24. The method according to claim 22 wherein the biotinylated proteins are bound to a streptavidin-coated substrate.

25. The method according to claim 24 wherein the substrate comprises a hydrogel-forming polymer base layer.

26. The method according to claim 20 wherein the antigens are exposed to a sample extracted from a person, such that autoantibody biomarkers from the sample may bind to the antigens.

27. The method according to claim 26 wherein the antigens are subsequently exposed to a fluorescently-tagged secondary antibody to allow the amount of any autoantibodies from the sample bound to the antigens to be determined.

28. The method according to claim 27 wherein the presence of non-small cell lung cancer corresponds to the relative or absolute amount of autoantibodies from the sample specifically binding to the antigens.

29. The method according to claim 20 wherein the steps are performed in vitro.

30. The method according to claim 20 wherein the method comprises detecting upregulation/downregulation of one or more of said biomarkers.

31. A method for manufacturing a kit for diagnosing Non-Small Cell Lung Cancer from a sample extracted from a subject, comprising the steps of:
for each antigen in a panel, cloning a biotin carboxyl carrier protein folding marker in-frame with a gene encoding the antigen and expressing the resulting biotinylated antigen;
binding the biotinylated proteins to addressable locations on one or more streptavidin-coated substrates, thereby forming an antigen array;
such that the amount of autoantibodies from the sample binding to the antigens on the panel can be determined by exposing the substrate to the sample and measuring the response;
characterised in that the antigens comprise XAGE1D, LRRFIP2 and GAGE2C.

32. The method according to claim 31 wherein the antigens further comprise one or more of DDX53, DDX43, GAGE1, MAGEA10, ZNRD1, MAP2K5, MAGEA4, STAT1, CT47A1, IGF2BP3, CTAG2, RAD23B, FADD, PTPN20A, TPM1, CTAG1A.
